(19) United States
(12) Patent Application Publication
(10) Pub. No.: US 2026/0023076 A1
(43) Pub. Date: Jan. 22, 2026

(54) METHODS OF DIAGNOSING OR TREATING LYME DISEASE

(71) Applicant: Drexel University, Philadelphia, PA (US)

(72) Inventors: Mary Comunale, Bangor, PA (US); Benjamin Haslund-Gourley, Santa Barbara, CA (US)

(21) Appl. No.: 19/116,648

(22) PCT Filed: Sep. 29, 2023
(86) PCT No.: PCT/US2023/034148

§ 371 Date: Mar. 28, 2025

Related U.S. Application Data
(60) Provisional application No. 63/412,292, filed on Sep. 30, 2022.
(60) Provisional application No. 63/444,693, filed on Feb. 10, 2023.

(51) Int. Cl. G01N 33/569; A61K 31/43; A61K 31/545; A61K 31/65; A61K 31/7052; A61P 31/04; G01N 33/68

(52) U.S. Cl. CPC G01N 33/56911; A61K 31/43; A61K 31/545; A61K 31/65; A61K 31/7052; A61P 31/04; G01N 33/6848; G01N 2333/20; G01N 2440/38; G01N 2469/20

(57) ABSTRACT

Described herein is a method of diagnosing Lyme disease in a subject. The method includes determining a glycosylation profile of a protein whose glycosylation profile is associated with Lyme disease in a subject, and comparing the glycosylation profile of the protein with a predetermined glycosylation profile of the protein indicating free of Lyme disease or a predetermined glycosylation profile of the protein indicating Lyme disease. Further described herein is a method of treating and/or ameliorating Lyme disease in a subject. The method includes diagnosing Lyme disease in a subject and, if the subject is diagnosed to have Lyme disease, administering to the subject an effective amount of compound effective for treating Lyme disease.

### CROSS-REFERENCE TO RELATED APPLICATIONS

The present application claims priority under 35 U.S.C. § 119(e) to U.S. Provisional Patent Application No. 63/412,292, filed Sep. 30, 2022 and U.S. Provisional Patent Application No. 63/444,693, filed Feb. 10, 2023, both of which are incorporated herein by reference in their entireties.

### BACKGROUND

There are approximately 476,000 cases of Lyme disease (LD) each year in the US. LD is diagnosed through a two-tier assay of serological reactivity towards Borrelia burgdorferi (Bb) antigens. This testing system has a low sensitivity of 46% and a specificity of 99%. Testing is highly dependent on the presence of an erythema migrans skin lesion (EM) that is present in only 70-80% of patients and may be difficult to detect on darker skin pigmentation. For those without EM presentation, diagnosis is almost impossible in the acute phase of disease. When LD is not treated within the acute phase, the spirochete disseminates into synovial, cardiac, and neuronal tissue, making eradication challenging and treatment more difficult. A subset of patients suffers from Post-Treatment Lyme Disease Syndrome (PTLDS), which is poorly elucidated due to a lack of diagnostic tools. Pregnant women who are acutely infected with Borellia and do not receive treatment have experienced multiple adverse pregnancy outcomes, including preterm delivery, stillbirth, and congenital cardiac malformations. Finally, infection does not generate an adaptive immune response to protect against future infections, although the patient often remains antibody positive. Hence, the current serological testing is unable to determine if an infection is a new infection, or the patient is positive due to a previous infection.

Several factors are leading to increased cases of LD including: expanding habitat due to reforestation and climate change, increased tick populations, and increased percent of infected ticks. Testing remains reliant on seroconversion and misses acute cases when treatment is most effective. Current tests are unable to identify active acute infections from past infections. The advantage of this method is not only improved sensitivity for acute LD diagnosis but also the ability to track disease resolution and differentiate between new infections, old infections, and mimic diseases.

Therefore, there is a need for effective Lyme disease diagnostic, prognostic, and treatment methods. The present invention addresses this need.

### SUMMARY

In some aspects, the present invention is directed to a method of treating, ameliorating and/or preventing acute Lyme disease in a subject.

In some embodiments, the method comprises determining a glycosylation profile of a protein in the subject. In some embodiments, a change of the glycosylation profile of the protein relative to a normal glycosylation profile is associated with Lyme disease.

In some embodiments, the method further comprises comparing the glycosylation profile of the protein in the subject with a predetermined first glycosylation profile indicating acute Lyme disease, or a predetermined second glycosylation profile indicating a state other than acute Lyme disease.

In some embodiments, the method further comprises administering to the subject a compound for treating, ameliorating and/or preventing acute Lyme disease if the determined glycosylation profile indicates acute Lyme disease.

In some embodiments, the change of the protein glycosylation profile comprises an increased level of glycosylation, or a decreased level of glycosylation.

In some embodiments, the method comprises determining a glycosylation profile of one or more purified proteins in the subject, and comparing the determined glycosylation profile with a first glycosylation profile of the one or more purified proteins indicating acute Lyme disease or a second glycosylation profile of the one or more purified proteins indicating states other than acute Lyme disease.

In some embodiments, the method comprises determining a total serum glycosylation profile in the subject, and comparing the determined total serum glycosylation profile with a first total serum glycosylation profile indicating acute Lyme disease or a predetermined second total serum glycosylation profile indicating states other than acute Lyme disease.

In some embodiments, the determined glycosylation profile, the first glycosylation profile and the second glycosylation profile comprise glycosylation profiles of a total serum, a total IgG, a total IgM, a Lyme-specific IgG, or a Lyme-specific IgM.

In some embodiments, the determined glycosylation profile, the first glycosylation profile and the second glycosylation profile comprise glycosylation profiles of a total serum or a purified IgG, and the change of the glycosylation profile comprises: (a) a decreased level of terminal agalactosylated and/or fucosylated structures, and/or (b) an increase in tri- or tetra-antennary, terminal galactose or sialic acid structures.

In some embodiments, the determined glycosylation profile, the first glycosylation profile and the second glycosylation profile comprises glycosylation profiles of purified IgM. In some embodiments the change of the glycosylation profile comprises (a) an increase in mannose, G0, G1 or G2, and/or (b) a decrease in bisecting and sialic acid containing structures.

In some embodiments, the change of the glycosylation profile comprises at least one IgG N-glycan selected from the group consisting of G0, G1, total G1, FA2G1 (1,6), core-fucosylated N-glycans, and FA2BG2S2 decreases in comparison with the normal IgG N-glycan.

In some embodiments, the change of the glycosylation profile comprises at least one IgG N-glycan selected from the group consisting of total G2, S2, FA1, FA2BG1 (1,3), FA2BG2, A12G2+Man 6, A2G2S1 (1,6), FA2G1S1 (1,6), A2G2S1 (1,3), FA2G2S1 (1,6), A2BG2S2, FA2BG2S2, and A2G2S2 (1,6) increases in comparison with the normal IgG N-glycan.

In some embodiments, the change of the glycosylation profile comprises at least one IgM N-glycan selected from the group consisting of total G2, FA2BG1S1 (1,3), S2, FA2BG2S2, bisecting N-glycans, A2G2S2 (3,6), and A2G2S2 (6,6) decreases in comparison with the normal IgM N-glycan.

In some embodiments, the change of the glycosylation profile comprises at least one IgM N-glycan selected from the group consisting of total G1, G1, G2, Total mannose N-glycans, Man 5, M4G1S1+A3G2, A1, FA1, G0, FA2G1, M4G1, FM4A1, M4A1G1, and Man 6 D1 or D2 increases in comparison with the normal IgM N-glycan.

In some embodiments, the change of the glycosylation profile comprises at least one total serum N-glycan selected from the group consisting of core-fucosylated N-glycans, G0, total G1, G1, FA2G1 (1,6), FA2G1 (1,3). FA2BG1 (1,6), G2, and FA2BG2 decreases in comparison with the normal total serum N-glycan.

In some embodiments, the change of the glycosylation profile comprises at least one total serum N-glycan selected from the group consisting of S2 and A2G2S2 (1,3) increases in comparison with the normal total serum N-glycan.

In some embodiments, the determined glycosylation profile of the protein in the subject is compared with at least one of a glycosylation profile indicating a healthy state, a glycosylation profile indicating a state of acute Lyme disease, a glycosylation profile indicating disseminated Lyme disease, a glycosylation profile indicating a state of recovering from Lyme disease, a glycosylation profile indicating a recovered case of Lyme disease, and a glycosylation profile indicating a non-Lyme infection or inflammatory disease.

In some embodiments, the method further comprises comparing the determined glycosylation profile with a glycosylation profile indicating a non-Lyme infection or inflammatory disease to exclude the non-Lyme infection or inflammatory disease. In some embodiments, the non-Lyme infection or inflammatory disease comprises at least one selected from the group consisting of fibromyalgia, lupus rheumatoid arthritis, and syphilis.

In some embodiments, the method determines the stage of Lyme disease in addition to the absence or presence of acute Lyme disease.

In some embodiments, the stage of Lyme disease includes early localized or acute Lyme disease, early disseminated Lyme disease, late disseminated Lyme disease, state of recovering from Lyme disease, or reinfection with Lyme disease.

In some embodiments, the method further comprises diagnosing the acute Lyme disease with the detection of Borrelia reactive antibodies in the subject.

In some embodiments, determining the glycosylation profile of the protein in the subject comprises at least one selected from the group consisting of: purifying the protein from a sample of the subject and releasing glycans from the protein, and releasing glycans from total serum proteins.

In some embodiments, the glycosylation profile of the protein in the subject and/or the predetermined glycosylation profiles are determined by a mass spectrometry method selected from the group consisting of: matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, matrix-assisted laser desorption/ionization imaging Fourier transform ion cyclotron resonance (MALDI-FT-ICR) mass spectrometry, Quadrupole ion trap (QIT) mass spectrometry, Linear Ion Trap (LIT) mass spectrometry, Orbitrap mass spectrometry, Magnetic sector mass analyzer.

In some embodiments, the glycosylation profile of the protein in the subject and/or the predetermined glycosylation profiles are determined by at least one selected from the group consisting of a high-pressure liquid chromatography (HPLC), and an ultra-low pressure liquid chromatography (UPLC).

In some embodiments, the glycosylation profile of the protein in the subject and/or the predetermined glycosylation profiles are determined by a capillary electrophoresis-based systems (CE).

In some embodiments, the glycosylation profile of the protein in the subject and/or the predetermined glycosylation profiles are determined by a microchip-based systems.

In some embodiments, the glycosylation profile of the protein in the subject and/or the predetermined glycosylation profiles are determined by a lectin or enzyme-linked immunosorbent assay (FLISA/ELISA)-based method, a microchip array method, or a western blotting method.

In some embodiments, the method diagnoses an acute Lyme disease in the subject before or after seroconversion.

In some embodiments, the compound comprises an antibiotic effective for killing a Borrelia bacteria.

In some embodiments, the antibiotic comprises at least one selected from the group consisting of doxycycline, amoxicillin, a cephalosporin, and azithromycin.

In some embodiments, the antibiotic is administered orally or parentally.

In some embodiments, comparing the glycosylation profile is combined with a C6 peptide ELISA or a Lyme disease IgM western immunoblot results when determining whether the subject is suffering from acute Lyme disease.

In some embodiments, determining whether the subject is suffering from actute Lyme disease comprises determining a total IgG glycome and a total IgM glycome, and combining the total IgG and total IgM glycomes with a C6 peptide ELISA or a Lyme disease IgM western immunoblot results.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1: a chart of patient samples and their source that are used in the present study, in accordance with some embodiments.

FIGS. 2A-2B: the glycan abbreviations and the grouping used in the diagnostic algorithm, in accordance with some embodiments.

FIG. 3: diagram illustrating certain aspects of analyzing N-glycans on IgM and IgG from acute Lyme disease serum revels lymphatic system immuno-modulation, in accordance with some embodiments.

FIGS. 4A-4B: illustrations of HPLC-FLR, UPLC-FLR-ESI-MS, Ab-capture MALDI-MS, MALDI-MS methodologies, which can be used to interrogate the diseases cohort glycosylation contemplated herein, such as compared to healthy controls, in accordance with some embodiments. Non-limiting examples of the diseases include acute Lyme disease.

FIGS. 5A-5B demonstrate that acute LD IgG and IgM N-glycan chromatograms significantly differ from healthy controls, in accordance with some embodiments. N-glycans were enzymatically released from total IgG (FIG. 5A) and total IgM (FIG. 5B) purified from patient serum. N-glycans were fluorescently tagged and separated using column chromatography. Each peak presented represents a species of N-glycan. N-glycans identities were confirmed by mass spectrometry. The overlaid N-glycan profile peaks that discriminate between acute LD and healthy controls are indicated with arrows with respective structures presented to the left of each arrow. Samples presented were derived from the CDC Research I serum panel.

FIGS. 6A-6D describe certain aspects of the relationships between the IgG glycosylation of acute Lyme disease patients and healthy controls, in accordance with some embodiments.

FIGS. 6A-6B: IgG N-glycosylation Volcano plot identified N-glycans from UPLC-FLR-ESI-MS that differ significantly during acute Lyme disease (n=43) compared to healthy controls (n=114) and are listed in FIG. 6B. FIGS. 6C-6D: The top 17 statistically significant N-glycans differing during acute Lyme disease are plotted in the graphs to the right, separated by high- and low-abundance. Data presented as means+/−standard deviation with statistical significance denoted from multiple t-tests by *p>0.05, **p>0.01, ***p>0.001.

FIGS. 7A-7D describe certain aspects of the relationships between IgM glycosylation in acute Lyme disease patients and healthy controls, in accordance with some embodiments. FIGS. 7A-7B: IgM N-glycosylation Volcano plot identified N-glycans from UPLC-FLR-ESI-MS that differ significantly during acute Lyme disease (n=43) compared to healthy controls (n=116) and are listed in FIG. 7B. FIGS. 7C-7D: The top 21 statistically significant N-glycans differing during acute Lyme disease are plotted in the graphs to the right, separated by high- and low-abundance. For IgM, the S1, S2 and Bisecting classes decrease. Conversely the G1, G2, and mannosylated N-glycans increase in abundance. Data presented as means+/−standard deviation with statistical significance denoted from multiple t-tests by *p>0.05, **p>0.01, ***p>0.001.

FIG. 8 describes certain aspects of the relationships between healthy controls and acute Lyme disease N-glycan biomarkers, in accordance with some embodiments. When IgG and IgM N-glycans are combined, there is a statistically significant difference between healthy control cohorts (n=114) and acute Lyme disease (n=43) were analyzed together, resulting in an N-glycan score that separated a majority of the acute Lyme disease cohort from healthy controls (left). The resulting ROC curve (right) is presented with an area under the curve (AUC) of 0.9028, sensitivity of 77%, and specificity of 87%.

FIG. 9 describes certain aspects of the relationships between primary infection and patients that have been reinfected, in accordance with some embodiments. IgG N-glycan profiles identify primary acute Lyme disease (naive) and Acute Lyme disease in patients with a previous history of Lyme disease. Healthy control n=18, acute 1st infection n=9, acute with previous infection n=9, standard error of the mean presented. Statistical analysis was performed using One-way ANOVA with Tukey's multiple comparisons test, **p<0.01

FIGS. 10A-10B describe certain aspects of the relationships between healthy control, acute Lyme disease, and treated/convalescent Lyme disease N-glycan biomarkers in IgG and IgM, in accordance with some embodiments. FIGS. 10A-10B compares IgG and IgM N-glycans (respectively) from healthy controls, acute Lyme disease, and matching re-draws from 16-18 of the acute Lyme disease patients after antibiotic treatment and 70-90 days of convalescence had occurred. In many cases, the N-glycans increased or decreased during acute Lyme disease resolve to healthy control-levels following treatment and convalescence. Data presented as means+/−standard deviation with statistical significance denoted from multiple t-tests by *p>0.05, **p>0.01, ***p>0.001.

FIG. 11 describes certain aspects of the relationships between acute Lyme disease state and ‘mimic’ disease N-glycan biomarkers in IgG and IgM, in accordance with some embodiments. ROC curves of acute Lyme disease (n=43) IgG and IgM N-glycans were compared to an n=20 from Lyme disease “mimic diseases” such as fibromyalgia, syphilis, lupus, and rheumatoid arthritis. The resulting ROC curves demonstrate acute Lyme disease can be discriminated from the mimic diseases with high accuracy as evidenced by the AUCs range of 0.81-0.92. Data presented as means+/−standard deviation with statistical significance denoted from student's t-test by **p>0.01.

FIGS. 12A-12B describe certain aspects of the relationships between acute Lyme disease state and healthy control N-glycan biomarkers in IgG and IgM, in accordance with some embodiments. FIG. 12A: IgG and IgM N-glycan scores discriminate EM (+) patients who test positive on the standard-two-tiered serology (STTT) assay from patients that are STTT negative. ROC curves from Control (n=114) vs Acute LD STTT (+) (n=29) result in an AUC of 0.93, sensitivity of 72%, and specificity of 89%. FIG. 12B: control (n=114) vs Acute LD STTT (−) (n=14) result in an AUC of 0.89, sensitivity of 64%, and specificity of 87%. Data presented as means+/−standard deviation with statistical significance denoted from multiple t-tests by *p>0.05, **p>0.01, ***p>0.001.

FIGS. 13A-13D describe certain aspects of the relationships between acute Lyme disease state and healthy control N-glycan biomarkers in serum, in accordance with some embodiments. FIGS. 13A-13B: total serum N-glycosylation volcano plot identified N-glycans from UPLC-FLR-ESI-MS that differ significantly during acute Lyme disease (n=41) compared to healthy controls (n=26). FIGS. 13C-13D: the top 9 statistically significant N-glycans differing during acute Lyme disease are plotted in the graphs to the right, separated by high- and low-abundance. For total serum N-glycans, the fucosylated, G0, G1, and G2 N-glycans decreased compared to healthy controls. Data presented as means+/−standard deviation with statistical significance denoted from multiple t-tests by *p>0.05, **p>0.01, ***p>0.001.

FIGS. 14A-14B describe certain aspects of the relationships between acute Lyme disease state, healthy control, and late-stage Lyme disease N-glycan biomarkers in IgG and IgM, in accordance with some embodiments. FIG. 14A. Comparison of IgG and IgM N-glycans from healthy controls (n-114), acute Lyme disease (n=43), and Lyme disease (n=4:2 Lyme arthritis patients and 2 Lyme Neuroborreliosis patients). During acute Lyme disease, IgG the G0 N-glycans decrease while in late-stage Lyme disease the G0 increases to levels higher than healthy controls indicating a significant change in disease response occurring during late-stage Lyme disease. Moreover, the larger G2, S1, and S2 N-glycans increase during acute Lyme disease while decreasing lower than healthy control levels in late-stage Lyme patients. FIG. 12B. IgM N-glycans S1 and Mannose increase during late-stage Lyme (n=4) compared to acute Lyme disease (n=116) while the S2, S3 and total G3 classes of N-glycans decreased in late-Lyme disease compared to acute Lyme disease. Data presented as means+/−standard deviation without statistical analysis due to the n=4 sample number for late-stage Lyme.

FIG. 15 lists the Lyme disease biobank (LDB) and precision for medicine biobank (PFM) cohort demographics in accordance with some embodiments. Positive standard two-tiered test (STTT) is defined by a positive or equivocal enzyme immunoassay (EIA) or immunofluorescence assay (IFA) followed by a positive IgG or IgM WB as defined by CDC criteria and described in Branda et al. 2021. n/a=data not provided by the biobank; EM=erythema migrans; WB=western immunoblot; POS=positive.

FIGS. 16A-16F demonstrate that IgG and IgM N-glycome discriminate Lyme-endemic healthy controls (LEHC), acute Lyme disease (ALD) patients, and treated Lyme disease (TLD) patients. Random forest method and cross-validation ROC with raw N-glycome data were used. FIG. 16A: IgG, IgM, or a combination of IgG and IgM N-glycans were used to discriminate between patients with acute Lyme disease (n=94) and healthy controls from Lyme-endemic areas (n=112), listing the significant N-glycans from the IgG and IgM N-glycome. FIG. 16B: C6 peptide ELISA results (positive, indeterminate, or negative) in combination with IgG and IgM N-glycan profiles were used to discriminate between patients with acute Lyme disease (n=78) and healthy controls from Lyme-endemic areas (n=100). FIG. 16C: IgM western immunoblot results (positive, indeterminate, or negative) in combination with IgG and IgM N-glycan profiles were used to discriminate between patients with acute Lyme disease (n=112) and healthy controls from Lyme-endemic areas (n=94). FIG. 16D: IgG, IgM, or a combination of IgG and IgM N-glycans were used to discriminate between patients with acute Lyme disease (n=94) and patients convalescing (treated) from Lyme disease (n=50), listing the significant predictors from the IgG and IgM N-glycome. FIG. 16E: C6 peptide ELISA results (positive, indeterminate, or negative) in combination with IgG and IgM N-glycan profiles were used to discriminate between patients with acute Lyme disease (n=78) and patients convalescing (treated) from Lyme disease (n=30). FIG. 16F: IgM western immunoblot results (positive, indeterminate, or negative) in combination with IgG and IgM N-glycan profiles were used to discriminate between patients with acute Lyme disease (n=112) and patients convalescing (treated) from Lyme disease (n=38). Area Under the Curve (AUC), Sensitivity (Sen.), and Specificity (Spc.) are presented for each ROC curve. DeLong's test was used to compare two ROC curves with **p<0.01 and ***p<0.001.

FIGS. 17A-17G demonstrate that Borrelia OspC- and VlsE-specific IgG combined with sialic acid abundance identifies acute Lyme disease patients, in accordance with some embodiments. FIG. 17A: Example of traditional detection of Borrelia burgdorferi (Bb) antigen-specific antibodies using a fluorescent reporter. FIG. 17B: Example of GlycoLyme method detects a multiplexed sialic acid content and anti-IgG signal in a Bb antigen-specific antibody population. FIG. 17C: Comparison of IgG abundance from non-Lyme endemic healthy controls (NLEHC) n=96, Lyme endemic healthy controls (LEHC) n=111, acute Lyme disease (ALD) n=89, treated Lyme disease (TLD) n=50, rheumatoid arthritis (RA) n=20, lupus (L) n=20, syphilis (S) n=20, and fibromyalgia (F) n=20. FIG. 17D: Comparison of GlycoLyme [anti-OspC/VlsE IgG*SNA] scores from non-Lyme endemic healthy controls (NLEHC) n=96, Lyme endemic healthy controls (LEHC) n=111, acute Lyme disease (ALD) n=89, treated Lyme disease (TLD) n=50, rheumatoid arthritis (RA) n=20, lupus (L) n=20, syphilis (S) n=20, and fibromyalgia (F) n=20. FIG. 17E: Receiver operating characteristic (ROC) curve compares ALD to non-ALD (NLEHC, LEHC, RA, L, S, F) with and without accounting for sialic acid abundance using the GlycoLyme method was analyzed with DeLong's test to determine statical significance with ***p<0.001. FIG. 17F: Comparison of n=35 GlycoLyme score positive ALD with matching TLD timepoint Bb-specific IgG abundance. FIG. 17G: Comparison of n=35 GlycoLyme score positive ALD with TLD time point Bb-specific immunoglobulin SNA fluorescence. Error bars present the mean+/−standard deviation (SD). Statistical significance was determined using an ordinary one-way ANOVA with Tukey's multiple comparisons test with ***p<0.001 for FIGS. 17A and 17B, while paired two-tailed student's t-test determined significance for FIGS. 17F and 17G with ****p<0.0001 and ns=not significant.

FIGS. 18A-18C demonstrate that sialic acid impairs VlsE-specific antibody-dependent cellular cytotoxicity (ADCC), in accordance with some embodiments. FIG. 18A: Example of the VlsE-specific ADCC assay method with and without exoglycosidase digestions created with BioRender. FIG. 18B: ADCC assay results expressed as a percent of the luminescent signal of the mock-exoglycosidase (Mock) compared to that of the PNGase F (PNG) or sialidase (SA) digested antibody ADCC activity. Pooled sets of sera were assayed from Lyme-endemic healthy control (LEHC) n=2, acute Lyme disease (ALD) n=4, and treated Lyme disease (TLD) n=2. FIG. 18C: Following ADCC assay detection, the plate-bound anti-VlsE IgG from LEHC (n=2), ALD (n=4), and TLD (n=2) was quantified using a fluorescent antibody reporter in triplicate. Statistical significance was determined using a nonparametric, repeated measures one-way ANOVA with Dunn's multiple comparisons test with *p<0.05, **p<0.01, n.d.=ADCC activity below the limit of detection, and n.s.=not significant.

FIGS. 19A-19C demonstrate that sialic acid impairs VlsE-specific antibody-dependent complement deposition (ADCD), in accordance with some embodiments. FIG. 19A: Example of the VlsE-specific ADCD assay method with and without exoglycosidase digestions. FIG. 19B: Example of gating strategy to detect ADCD using flow cytometry. FIG. 19C: VlsE-specific IgG complement deposition presented as a ratio of the Lyme-endemic healthy control (LEHC) n=2 complement deposition to either the acute Lyme disease (ALD) n=4 or treated Lyme disease (TLD) n=2 complement deposition. Samples were assayed in duplicates and statistical significance was determined using a one-way ANOVA with Dunn's multiple comparisons test with *p<0.05. The precent change between mock-digested and sialidase-digested sets of LEHC, ALD, and TLD are indicated in the graph. The PBS background is noted by the dashed line.

FIGS. 20A-20B illustrate the N-glycan profiles of IgM and IgG isolated from human sera, in accordance with some embodiments. FIG. 20A: IgM N-glycans labeled with the RapiFluor (RFMS) were profiled with UPLC-FLR-ESI-MS platform. The resulting N-glycans were identified using mass spectrometry and retention time data. IgM N-glycans are grouped by class: G0 refers to core diantennary N-glycans lacking galactose, G1 refers to core diantennary N-glycans with a single galactose, G2 refers to core diantennary N-glycans with two galactoses, S1 refers to diantennary N-glycans with a single sialic acid, S2 refers to di- and tri-antennary N-glycans with two sialic acids, S3 refers to triantennary N-glycans with three sialic acids, Mannose refers to M4-M10 and hybrid-type N-glycans, Fucose refers to any N-glycan with a core-fucose, Bisecting refers to any N-glycan with a bisecting GlcNAc moiety. FIG. 20B: IgG N-glycans are grouped by class: G0 refers to core diantennary N-glycans lacking galactose, G1 refers to core diantennary N-glycans with a single galactose, G2 refers to core diantennary N-glycans with two galactoses, S1 refers to diantennary N-glycans with a single sialic acid, S2 refers to diantennary N-glycans with two sialic acids, Bisecting refers to any N-glycan with a bisecting GlcNAc moiety, Fucose refers N-glycans with a core-fucose.

FIGS. 21A-21D demonstrate that IgG and IgM N-glycome discriminate acute Lyme disease (ALD) patients from autoimmune and infectious diseases using the random-forest machine learning method on cross-validation, in accordance with some embodiments. FIG. 21A: IgG, IgM, or a combination of IgG and IgM N-glycans were used to discriminate between patients with acute Lyme disease (n=94) and patients with lupus (n=18). FIG. 21B: IgG, IgM, or a combination of IgG and IgM N-glycans were used to discriminate between patients with acute Lyme disease (n=94) and patients with fibromyalgia (n=37). FIG. 21C: IgG, IgM, or a combination of IgG and IgM N-glycans were used to discriminate between patients with acute Lyme disease (n=94) and patients with syphilis (n=20). FIG. 21D: IgG, IgM, or a combination of IgG and IgM N-glycans were used to discriminate between patients with acute Lyme disease (n=94) and patients with rheumatoid arthritis (n=20). Area Under the Curve (AUC), Sensitivity (Sen.), and Specificity (Spc.) are presented for each ROC curve. DeLong's test compared two ROC curves with *p<0.05 and ***p<0.001.

FIG. 22 illustrates certain aspects of Bb Antigen Selection. Pools of sera from non-Lyme endemic healthy controls (NLEHC) n=2, Lyme endemic healthy controls (LEHC) n=2, acute Lyme disease (ALD) n=4, treated Lyme disease (TLD) n=2, rheumatoid arthritis (RA) n=2, lupus (L) n=2, syphilis (S) n=2, and fibromyalgia (F) n=2 were assayed using the GlycoLyme score approach [IgG*SNA] to determine which antigen(s) bound to the plates at 50 ng/well yielded the highest area under the curve (AUC).

FIG. 23 illustrates certain aspects of the validation of sialidase digestion on human samples. Sialidase-digested (SA) pools of LEHC n=2, ALD n=4, and TLD n=2 sera were compared to mock-digested (Mock) pools, respectively, with percent of signal reduction noted for each cohort.

FIG. 24: Cross-validation AUC collected from total IgG and IgM N-glycans (raw data or log ratios combined with raw data) determined by least absolute shrinkage and selection operator (Lasso), Naïve Bayes (NB), Support vector machine (SVM), Random forest (RF), Gradient Boosting Machine (GBM), or Extreme Gradient Boosting Trees (XGBOOST), in accordance with some embodiments.

FIG. 25: LEHC vs ALD top 30 important IgG and IgM N-glycan predictors determined by two-sample t-test, presenting mean+/−standard deviation (SD), in accordance with some embodiments.

FIGS. 26A-26B: ALD vs TLD top 30 important IgG and IgM N-glycan predictors determined by paired t-test, presenting mean+/−standard deviation (SD) and (%) change from ALD to TLD, in accordance with some embodiments.

FIG. 27: ALD vs lupus top 30 important IgG and IgM N-glycan predictors determined by two-sample t-test, presenting mean+/−standard deviation (SD), in accordance with some embodiments.

FIGS. 28A-28B: ALD vs fibromyalgia top 30 important IgG and IgM Nglycan predictors determined by two-sample t-test, presenting mean+/−standard deviation (SD), in accordance with some embodiments.

FIGS. 29A-29B: ALD vs syphilis top 30 important IgG and IgM N-glycan predictors determined by two-sample t-test, presenting mean+/−standard deviation (SD), in accordance with some embodiments.

FIG. 30A-30B: ALD vs rheumatoid arthritis top 30 important IgG and IgM N-glycan predictors determined by two-sample t-test, presenting mean+/−standard deviation (SD), in accordance with some embodiments.

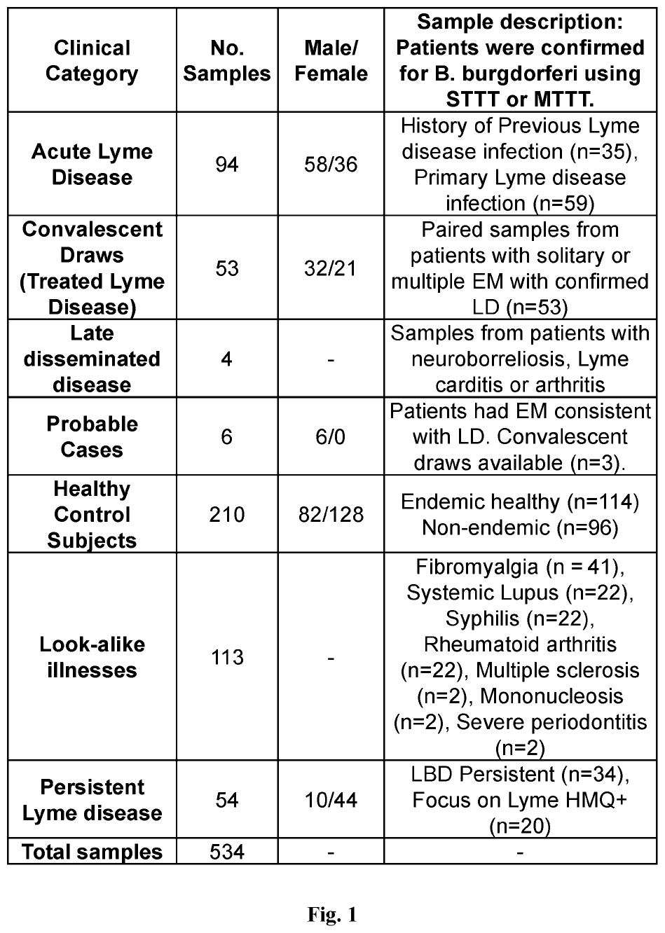

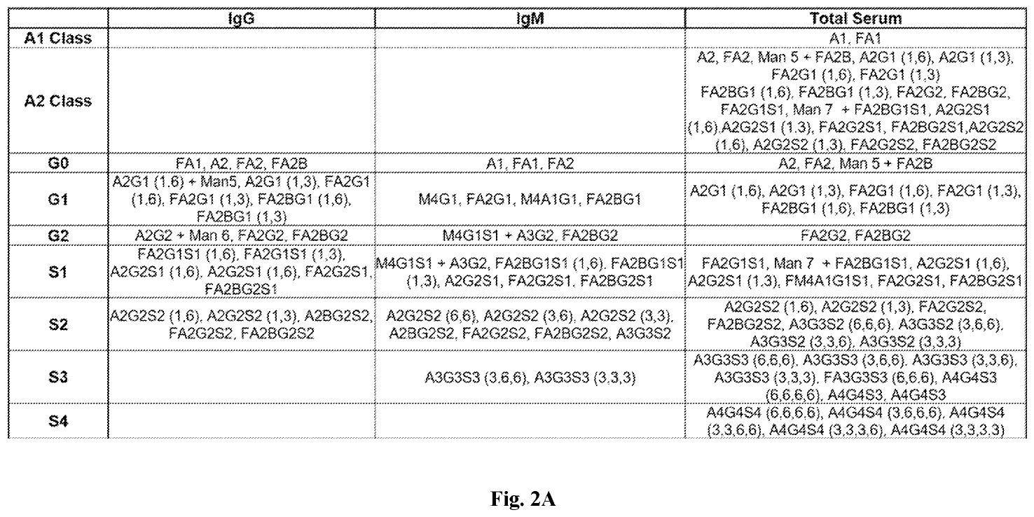

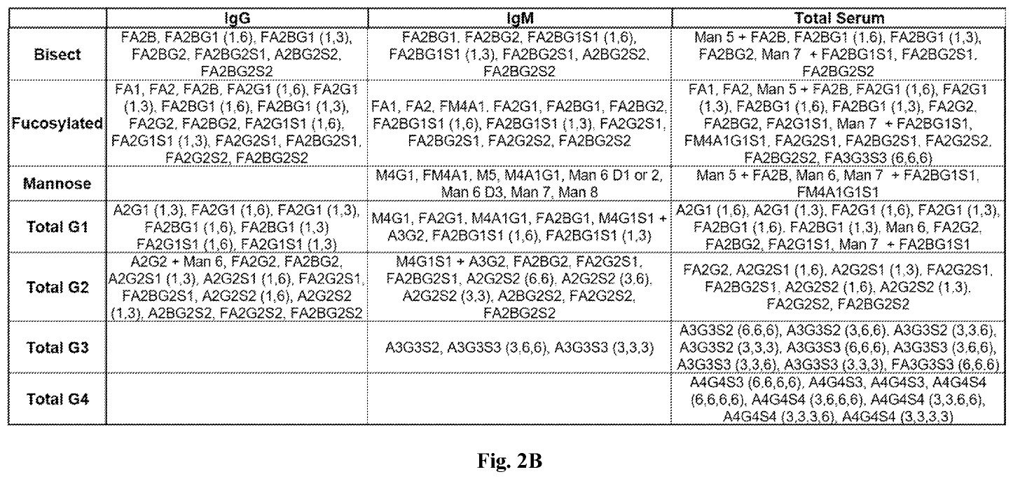

### DETAILED DESCRIPTION

The following disclosure provides many different embodiments, or examples, for implementing different features of the provided subject matter. Specific examples of components and arrangements are described below to simplify the present disclosure. These are, of course, /merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Currently, acute Lyme disease patients are often diagnosed on clinical grounds, such as for example based on the presence of an erythema migrans (EM) rash. Oftentimes, however; EMs are atypical, unnoticed, or completely absent in the patient. Thus, diagnosis of early Lyme disease is difficult for physicians and is frustrating for patients, particularly in the early stage of the disease such as the first month. When acute Lyme disease is not treated in the early stage, the spirochete disseminates to the joints, brain, and heart, resulting in chronic disability and/or potential loss of life.

Early diagnosis and treatment can effectively avoid the long-term disabilities that are commonly observed with Lyme infections. Early diagnosis of the disease, however, is not currently viable. In responding to the need in the art, the study described herein (“the present study”) developed a diagnostic test to detect active Lyme disease infection and/or distinguish it from certain common infections or inflammatory diseases with similar symptoms. In certain embodiments, the diagnostic test employs high sensitivity biomarkers. In certain embodiments, the diagnostic test enables early Lyme disease diagnosis, allows for early treatment of Lyme disease, and is useful in tracking the effectiveness of treatments.

A non-limiting version of the glycoproteomic based test according to the present invention, referred to herein as “GlycoLyme,” works by identifying unique changes to the sugars attached to proteins in patients, such as but not limited to immunoglobulins. These covalently N-linked glycans participate in modulating immune response. The present study discovered that patients with acute Lyme disease contain proteins that have significantly altered levels of mannose, galactose, and sialic acid content in the proteins, resulting in larger, more negatively charged glycans compared to healthy controls. In certain embodiments, the GlycoLyme test quantitates and identifies glycans from total serum Immunoglobulin G and M using fluorescent chromatography and mass spectrometry, respectively, or by plate-based assays. If patients' glycan profiles meet GlycoLyme's algorithmic threshold, the patient is deemed positive for acute Lyme disease.

The present study confirmed that the GlycoLyme test is highly accurate and can distinguish acute Lyme disease from related diseases or diseases with similar symptoms. As tested in the present study, GlycoLyme has a sensitivity of 72% in EM (+), STTT (+) patients This increased sensitivity allows for better detection of acute cases, allowing for patients to start treatments in a timelier fashion. Importantly, the assay can also identify patients who have the EM rash but are not yet seropositive according to EIA or STTT tests. GlycoLyme retains 80-100% specificity when tested using endemic and non-endemic healthy controls. Importantly, GlycoLyme can discriminate rheumatoid arthritis, syphilis, mononucleosis, and lupus from acute Lyme disease. Furthermore, GlycoLyme can distinguish Lyme neuroborreliosis and Lyme arthritis from acute Lyme disease, because samples of patients suffering from late-stage Lyme disease contain a different glycan profile compared to acute Lyme disease. As such, the GlycoLyme approach significantly improves testing accuracy, avoids false positives from mimic diseases, and offers a method to measure treatment efficacy.

The present study further confirmed that GlycoLyme test can be used to determine effectiveness of treatment for Lyme disease. Immunoglobulin glycans are dynamic and reflect the immune state. The GlycoLyme test identifies N-glycan structures significantly elevated in acute Lyme disease that resolve to healthy-control N-glycan levels in patients who returned to donate blood after remaining symptom-free during convalescence. Thus, patients and clinicians can confirm that the treatment for acute Lyme disease worked, by re-testing their blood using the GlycoLyme test to track their immune response over time.

Accordingly, in some embodiments, the present invention is directed to a method of diagnosing Lyme disease.

In some embodiments, the present invention is directed to a method of treating, ameliorating, and/or preventing Lyme disease.

In some embodiments, the present invention is directed to a method of determining an effectiveness of a treatment for Lyme disease.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in immunology, proteomic, glycomic, and glycoproteomics, protein and peptide chemistry, and organic chemistry are well-known and commonly employed in the art. It should be understood that the order of steps or order for performing certain actions is immaterial, so long as the present teachings remain operable. Any use of section headings is intended to aid the reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

Unless defined otherwise, all technical and scientific terms used herein have the same meaning as commonly understood by one of ordinary skill in the art to which the instant specification pertains. Although any methods and materials similar or equivalent to those described herein can be used in the practice for testing of the instant specification, selected materials and methods are described herein. In describing and claiming the instant specification, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably +5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the specification with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the specification within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the specification, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the specification, and are physiologically acceptable to the patient. Supplementary active compounds may also be incorporated into the compositions. The “pharmaceutically acceptable carrier” may further include a pharmaceutically acceptable salt of the compound useful within the instant specification. Other additional ingredients that may be included in the pharmaceutical compositions used in the practice of the instant specification are known in the art and described, for example in Remington's Pharmaceutical Sciences (Genaro, Ed., Mack Publishing Co., 1985, Easton, PA), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses therapy. Accordingly, the compositions and methods of the instant specification include therapeutic applications. Therefore “treating” or “treatment” of a state, disorder or condition includes: (i) inhibiting the state, disorder or condition, i.e., arresting or reducing the development of the disease or at least one clinical or subclinical symptom thereof, and/or (iii) relieving the disease, i.e. causing regression of the state, disorder or condition or at least one of its clinical or subclinical symptoms.

As used herein, the term “prevention” or “preventing” encompasses prophylaxis. Accordingly, the compositions and methods of the instant specification include prophylactic applications. Therefore prevention” of or “preventing” a state, disorder or condition includes preventing or delaying the appearance of clinical symptoms of the state, disorder or condition developing in a subject that may be afflicted with or predisposed to the state, disorder or condition but does not yet experience or display clinical or subclinical symptoms of the state, disorder or condition.

Ranges: throughout this disclosure, various aspects can be presented in a range format. It should be understood that the description in range format is merely for convenience and brevity and should not be construed as an inflexible limitation on the scope of the instant specification. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Abbreviations: Bb: Borrelia burgdorferi or Borrelia burgdorferi sensu lato. CE: capillary electrophoresis. DESI: desorption electrospray ionization. EIA: enzyme immunoassay. ELISA: enzyme linked immunosorbent assay. EM: erythema migrans. ESI: electrospray ionization. FLISA: fluorescent immunoassay. HPLC: high-performance liquid chromatography. LC: liquid chromatography. LIT: linear ion trap. LD: Lyme disease. MALDI: matrix-assisted laser desorption/ionization. MALDI FT-ICR: matrix-assisted laser desorption/ionization Fourier transform ion cyclotron resonance. MALDI-TOF: matrix-assisted laser desorption/ionization time of flight. MS: mass spectrometry. PTLDS: post-treatment Lyme disease syndrome. QIT: quadrupole ion trap. SALDI: scanning microprobe MALDI mass spectrometry. SIMS: secondary ion mass spectrometry. SMALDI: scanning microprobe MALDI. STTT: Standard Two-Tiered Test. UPLC: ultra-performance liquid chromatography.

Glycan abbreviations are as follows: A1 Class: glycans with a single-antennary glycan arm. A2 Class: glycans containing bi-antennary glycan arms. G0: glycan structures that lacking terminal galactose moieties. G1: glycan structures that containing one terminal galactose moiety. G2: glycan structures that containing two terminal galactose moieties. G3: glycan structures that containing three terminal galactose moieties. G4: glycan structures that containing four terminal galactose moieties. S1: glycan structures that containing one terminal sialic acid moieties. S2: glycan structures that containing two terminal sialic acid moieties. S3: glycan structures that containing three terminal sialic acid moieties. S4: glycan structures that containing four terminal sialic acid moieties. Bisecting: glycans containing a bisecting N-acetylglucosamine in complex asparagine lined glycans. Total G1: Glycans containing a single galactose moiety. Total G2: Glycans containing two galactose moieties. Total G3: Glycans containing three galactose moieties. Total G4: Glycans containing four galactose moieties.

### Method of Diagnosing Lyme Disease

The present study discovered that the samples obtained from acute Lyme disease patients have a unique set of protein glycosylation profiles, which distinguishes from those obtained from subjects that do not have acute Lyme diseases. For example, the present study discovered that the protein glycosylation profiles obtained from acute Lyme disease patients are different from those obtained from healthy subjects, subjects that have recovered from Lyme disease, or subjects suffering from certain infectious or inflammatory diseases that show symptoms similar to those found in acute Lyme disease. As such, the present study confirmed that the protein glycosylation profiles of samples obtained from patients can be used to diagnose acute Lyme disease.

Accordingly, in some aspects, the present invention is directed to a method of diagnosing acute Lyme disease.

In some embodiments, the method comprises determining a glycosylation profile of a protein in the subject, wherein a change of the glycosylation profile of the protein relative to a normal glycosylation profile is associated with Lyme disease; and comparing the glycosylation profile of the protein in the subject with a predetermined first glycosylation profile indicating acute Lyme disease, or a predetermined second glycosylation profile indicating a state other than acute Lyme disease.

In some embodiments, the term “Lyme disease” refers to the pathologies caused by or involves Borrelia infection, such as but not limited to a Borrelia burgdorferi infection, such as but not limited to a Borrelia burgdorferi sensu stricto infection.

In some embodiments, the term “acute Lyme disease” refers to the early stage of localized disease caused by or involves a Borrelia infection. Symptoms with early localized (or “acute”) Lyme disease may begin hours, a few days, or even weeks after a tick bite. At this stage, the infection has not yet spread throughout the body and/or formed biofilms that are resistant to antibiotic treatment. Treatments of Lyme disease with antibiotics at this stage has significantly higher chance of curing the disease. In contrast, in the later stages of Lyme disease, the bacteria are beginning to spread throughout the body (“early disseminated Lyme disease”) or have spread throughout the body (“late disseminated Lyme disease”). At these stages, the bacteria have started to form or have already formed antibiotic-resistant biofilms. Treatments with antibiotics at these stages are less effective.

In some embodiments, the sample for determining glycosylation profile is a biological fluid, such as a serum. In some embodiments, the sample is from a subject suspected of having been infected with Borrelia spirochete, which can be used for diagnosing the subject. In some embodiments, the sample is from a subject confirmed to have acute Lyme disease, confirmed to be healthy, confirmed to have been treated for Lyme disease, confirmed to have recovered from Lyme disease, or confirmed to have a non-Lyme infectious, inflammatory disease, or a disease that has symptoms or a clinical presentation similar to Lyme disease, such that the sample can be used to establish a reference glycosylation profile, such as the first glycosylation profile or the second glycosylation profile.

In some embodiments, the sample is used directly for determining the protein glycosylation profile. In some embodiments, the sample is a serum sample, and the sample is used for determining a total serum glycosylation profile.

In some embodiments, the sample is further processed to purify one or more proteins such that the glycosylation profile can be determined for the specific proteins.

The method of determining the glycosylation profile of the protein in the subject or constructing glycosylation profiles of Lyme disease, non-Lyme infections or inflammatory diseases, recovered Lyme patients, and healthy subjects are not limited.

In some embodiments, the protein of interest is purified from the sample. In some embodiments, all the proteins in the sample are proteins of interest, or total serum analysis can eliminate the need to purify proteins and/or removing glycans from proteins). In some embodiments, the protein is purified by affinity chromatography. In some embodiments the protein is purified by precipitation. In some embodiments, the protein is purified by solid phase chromatography, binding to beads or other solid matrix. In some embodiments, the glycans are released from the protein before the glycosylation profile is determined. In some embodiments, the glycans are not released from the protein and the profiles are determined with glycans attached to the protein. In some embodiments, the protein is digested into peptides and the glycan profiles are determined with the glycans remaining attached to the peptide.

The method of determining glycosylation profile of a protein is not limited. In some embodiments, the glycosylation profiles are determined by a mass spectrometry method selected from the group consisting of matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, matrix-assisted laser desorption/ionization imaging Fourier transform ion cyclotron resonance (MALDI-FT-ICR) mass spectrometry, Quadrupole ion trap (QIT) mass spectrometry, Linear Ion Trap (LIT) mass spectrometry, Orbitrap mass spectrometry, Magnetic sector mass analyzer, High Pressure Liquid Chromatography (HPLC), Ultra-Low Pressure Liquid Chromatography (UPLC) used alone or coupled with a selected mass spectrometry method, capillary electrophoresis-based systems (CE) used alone or coupled with a selected mass spectrometry method, microchipped based systems, a lectin or enzyme-linked immunosorbent assay (FLISA/ELISA)-based method, microchip array methods, or western blotting methods that that incorporate direct or indirect detection methods.

In some embodiments, the detection can be through any suitable method, including plate-based assays, ELISA/FLISA, western blot assays, microchip assays, microarrays, capillary electrophoresis, chromatography, or mass spectrometry. Detection may be through a labeled or unlabeled glycosyl moiety. The detection reagent can directly label the glycosyl moieties, for example, via carbohydrate-specific chemicals, labels, dyes, or using a labeled carbohydrate binding protein such as a lectin, immunoglobulin, or other suitable binding agent. Detection can be through labeling the protein or peptide attached to the glycosyl moieties, for example, by first capturing the target glycan analyte with a lectin and then binding the protein portion with an antibody. Detection can be through a secondary reagent, for example by first capturing the target analyte and then contacting the capture reagent-target complex with a labeled secondary reagent. Detection can be through immobilizing antigen(s) and capturing the target analyte, and then contacting the target analyte with a labeled secondary reagent. Detection can proceed by separating glycosyl moieties from the proteins prior to the quantifiable detection of glycosylation. Detection can proceed by enzymatically digesting the protein and identifying the glycopeptide. Detection can proceed by separating glycoproteins from the test sample before the quantifiable detection of glycosylation.

In some embodiments the protein used for profiling, such as the purified protein used for profiling, comprises at least one selected from the group consisting of Immunoglobulin G (IgG) and Immunoglobulin M (IgM), including all subclasses, for example, IgG1, IgG2, IgG3, IgG4, and/or IgM J chains.

In some embodiments, the Borrelia antigen protein or peptide(s) are immobilized to a suitable substrate (e.g. nitrocellulose membrane) at the test manufacturing site. In certain embodiments, the peptides are attached to or immobilized on a solid support, for example, a bead (e.g. colloidal particle, latex bead, etc.), a flow path in a lateral flow immunoassay device, a flow path in an analytical rotor, or a tube or well (e.g. plate). In certain embodiments, the protein or peptide mixture is attached to a dendrimer and/or incorporated into a MAPS system. In certain embodiments, the kits contain beads or a plate (e.g. dot blot, ELISA or FLISA assay). In other embodiments, the kits comprise a device such as a lateral flow immunoassay, analytical rotor, electrochemical sensor, optical sensor, or optoelectronic sensor. In certain embodiments, the beads, plate, or device detect the formation of antibody-peptide/protein complexes or lectin-antibody-peptide/protein complexes. In certain embodiments, the kit contains a peptide, protein, or mixture of different peptides and/or proteins. The sample is in contact with a single or mixture of two, three, four, or more Borrelia antigens. In certain embodiments, the kits contain instructions on using the population of beads, plates, or devices to detect naturally occurring antibodies to Borrelia antigens or glycans of proteins bound to Borrelia antigens (e.g. IgG, IgM). In certain embodiments, the kit combines labeled protein reporters and glycan reporters to detect the invention's protein and/or carbohydrate changes. The reporters can be labeled with a direct detection method (e.g. fluorophores) or an indirect detection method (e.g. biotin-enzyme reporters). In some embodiments, the methods comprise contacting a sample with the bound Borrelia antigen and detecting the antibody-antigen complex and the antibody glycan signature identified in the invention.

In the present study, glycosylation profiles of IgG, IgM, and total serum proteins (for example, as assayed by total serum glycome N-glycans) were studied in samples from Lyme disease patients of different disease stages, as well as samples from patients with non-Lyme disease patients, patients with diseases that have clinical presentations that ‘mimic’ Lyme disease, or healthy subjects. Such glycosylation profiles were confirmed to be useful for diagnosing Lyme disease, including establishing the stages of the Lyme disease. Accordingly, in some embodiments, the protein includes IgG, IgM, or total serum proteins.

The present study further identified that IgM and IgG specific for BorreliaLyme antigens are useful for diagnosing acute Lyme disease using glycosylation profiles. Accordingly, in some embodiments, the protein includes LymeBorrelia-specific IgG or LymeBorrelia-specific IgM. In some embodiments, the BorreliaLyme-specific immunoglobulin have specificity against antigens such as Bb antigens OspC, VlsE and etc.

In some embodiments, the measure of protein glycosylation levels/patterns shows an increased complexity of sugar structure with decreased fucosylation and/or terminal galactose structures and an increase in, antennary, terminal galactosylated, and/or sialic acid N-glycan structures.

In some embodiments, the glycosylation profile is a profile of IgG glycosylation or a total serum glycosylation profile, the change of the glycosylation profile includes: (a) a decreased level of terminal agalactosylated and/or fucosylated structures, and/or (b) an increase in tri- or tetra-antennary, terminal galactose or sialic acid structures.

In some embodiments, the glycosylation profile is a profile of IgM glycosylation, and the change of the glycosylation profile includes (a) an increase in mannose, G0, G1 or G2, and/or (b) a decrease in bisecting and sialic acid containing structures.

In some embodiments, IgG N-glycans that decrease during acute Lyme disease are: G0, G1, total G1, FA2G1 (1,6), core-fucosylated N-glycans, and FA2BG2S2. The IgG N-glycans that increase during acute Lyme disease are: Total G2, S2, FA1, FA2BG1 (1,3), FA2BG2, A12G2+Man 6, A2G2S1 (1,6), FA2G1S1 (1,6), A2G2S1 (1,3), FA2G2S1 (1,6), A2BG2S2, FA2BG2S2, and A2G2S2 (1,6).

In some embodiments, IgM N-glycans that decrease during acute Lyme disease are: Total G2, FA2BG1S1 (1,3), S2, FA2BG2S2, bisecting N-glycans, A2G2S2 (3,6), A2G2S2 (6,6). The IgM N-glycans that increase during acute Lyme disease are: Total G1, G1, G2, Total mannose N-glycans, Man 5, M4G1S1+A3G2, A1, FA1, G0, FA2G1, M4G1, FM4A1, M4A1G1, and Man 6 D1 or D2.

In some embodiments, total serum N-glycans that decrease during acute Lyme disease are: core-fucosylated N-glycans, G0, total G1, G1, FA2G1 (1,6), FA2G1 (1,3). FA2BG1 (1,6), G2, and FA2BG2. The total serum N-glycans that increase during acute Lyme disease are: S2 and A2G2S2 (1,3).

In some embodiments, the N-glycans isolated from IgG, IgM, and total serum listed above can be combined to discriminate between acute Lyme disease patients and healthy controls with higher sensitivity and specificity than any single source of glycans alone.

The presence of anti-Borrelia antibody, such as an anti-Borrelia burgdorferi antibody, indicates that the subject is: in the late phase of acute Lyme disease, in early or late disseminated Lyme disease, recovering from Lyme disease, having an acute Lyme disease from the second or subsequent infection, or has recovered from Lyme disease. Further, the absence of the antibody may indicate that the subject has never suffered from Lyme disease, or is suffering from the first-time acute Lyme disease, or was treated for a previous infection prior to seroconversion. Thus, the presence/titer or absence of anti-Borrelia antibodies can provide useful information to increase the accuracy of the present glycosylation profile-based method. Accordingly, in some embodiments, the method further comprises detecting an anti-Borrelia antibody in the subject.

The present study discovered that the method herein is able to detect acute Lyme disease before, as well as after, anti-Borrelia antibodies are detectable in the serum (pre-seroconversion). Accordingly, in some embodiments, the method diagnoses an acute Lyme disease in the subject before or after seroconversion.

Referring to Tables 6-10, 16-20 and 26-30, the present study further identified the differences in glycosylation profiles between acute Lyme disease samples and samples of rheumatoid arthritis, syphilis, lupus and fibromyalgia—diseases that sometimes show similar symptoms to those found in acute Lyme diseases. Accordingly, in some embodiments, the diagnosis of acute Lyme disease includes excluding the possibility of a mimic disease of acute Lyme disease, such as rheumatoid arthritis, syphilis, lupus, and fibromyalgia.

In some embodiments, the subject is a mammal, such as a human.

Method of Treating, Ameliorating, and/or Preventing Lyme Disease

The present study developed a novel approach of diagnosing Lyme disease, which can diagnose Lyme disease at acute stage before seroconversion. Since the treatments for Lyme disease are significantly more effective at acute Lyme disease stage than later stages, in some aspects, the present invention is directed to methods of treating, ameliorating, and/or preventing Lyme disease.

In some embodiments, the method of treating, ameliorating, and/or preventing Lyme disease includes: determining a glycosylation profile of a protein in the subject, wherein a change of the glycosylation levels/patterns of the protein in relative to a normal glycosylation profile is associated with Lyme disease; comparing the glycosylation profile of the protein in the subject with a predetermined first glycosylation profile indicating free of Lyme disease or a predetermined second glycosylation profile indicating Lyme disease; and if the glycosylation profile of the protein in the subject does not correspond to the first glycosylation profile or the glycosylation profile of the protein in the subject correspond to the second predetermined level, administering to the subject an effective amount of compound effective for treating, ameliorating, and/or preventing Lyme disease.

In some embodiments, the step of determining a glycosylation profile of a protein in the subject, the step of comparing the glycosylation profile of the protein in the subject with the first/second glycosylation profile, as well as establishing the first/second glycosylation profiles, are the same as or similar to those described elsewhere herein, such as in the “Method of Diagnosing Lyme disease” section.

In some embodiments, the compound includes an antibiotic effective for killing a Borrelia bacteria.

In some embodiments, the antibiotic includes at least one selected from the group consisting of doxycycline, amoxicillin, a cephalosporin (such as ceftriaxone, cefotaxime, or cefuroxime axetil) and azithromycin.

In some embodiments, the antibiotic is administered orally or intravenously.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: State of Human Lyme Borreliosis and the Potential Application of Glycome Analysis

Cases of Lyme Borreliosis have steadily increased over the last four decades. When the disseminated disease is not diagnosed and treated in a human host, the long-term effects are devastating. Strengths and shortcomings of current Lyme Borreliosis diagnostics are reviewed. The application of glycan analysis is introduced as a novel method to potentially diagnose and characterize Lyme Borreliosis. Glycosylated acute-phase proteins which are altered during early Lyme Borreliosis are examined. Lastly, characterization of the glycosylation pattern of total serum protein glycans, and IgM and IgG glycans is discussed as markers of response-to-treatment.

### Example 1-1: The Rise of Lyme

In North America, Europe, and Asia, Lyme disease is caused by the transmission of Borrelia burgdorferi sensu lato species complex via the feeding of Ixodes spp. ticks to the human host. The earliest known case of Lyme disease was confirmed by genome sequencing in a 5,300-year-old individual in the Italian part of the Ötztal Alps. More recently, Lyme disease was identified in the US during the late 1970s in association with unexplained cases of childhood arthritis. The causative agent of the arthritis was determined to be Borrelia burgdorferi sensu stricto (Bb). The rise in Lyme disease has been attributed in part to the reintroduction of forests and deer into the Northeast and Midwest US during the mid-20^{th} century. Case counts continue to rise; with over 20,000 annual reported cases of Lyme disease by 2010. A decade later, Lyme disease is the most common vector-borne disease in the US and totals an estimated 476,000 cases annually. While a majority of the Lyme disease cases are still located within the Northeast and upper-Midwest regions, ecological and environmental changes are promoting a gradual geographic expansion.

### Example 1-2: Etiology and Pathology of Lyme Disease

Bb is a highly motile gram-negative extracellular pathogen. One third of the Bb genome exists as linear and circular plasmids—making the species contain the most complex genomes among the known bacteria. During the acute phase infection in humans, Bb activates a proinflammatory Th1 response via interferon alpha and Toll-like receptor 2 signaling pathways. Bb encodes proteins that promote avoidance of compliment mediated lysis, antigenic variation of VIsE and outer surface protein C (OpsC), and adhesion to host decorin, glycosaminoglycans, and fibronectin. Bb has a slow growth rate due to its dependance on glycolysis to produce ATP and a requirement to scavenge choline phospholipids from host cell membranes. Human host cell metabolism of arachidonic acids, glycolysis, and glycolipids are altered during Lyme disease.

The acute stage of Lyme disease occurs within 1-2 weeks post-exposure with an erythema migrans skin rash (EM) identified in roughly half of infected individuals. Moreover, the classic Bull's Eye Rash EM is present in 9% of Lyme disease cases, while roughly 50% of Lyme disease patients will present with a ‘diffusely homogenous red plaque or patch’ EM. Independent of EM, patients will be either asymptomatic or present with headache, arthralgias, and fever. Dendritic cells located within the dermis phagocytose and present Bb antigens to T-cells for production of antibodies early during disease progression. Untreated infections will disseminate over the following weeks to months leading to a wide array of symptoms including bilateral Bell's facial nerve palsy, Lyme carditis, Lyme arthritis, and Lyme neuroborreliosis. In addition, pregnant women often lack a clear serodiagnostic profile. Thus, in untreated pregnant women, the Bb spirochete has been documented to cause myriad congenital Lyme disease-associated developmental disorders and fetal demise.

### Example 1-3: Diagnosis

Prior to laboratory testing for Lyme disease, physicians will screen for high pretest probability by collecting a detailed exposure history as well as patient signs and symptoms such as headache or arthralgias. The pre-test screening reduces the rate of false positives during diagnosis. Patients who live in or have traveled to a Lyme disease-endemic area presenting with an EM may be diagnosed and will begin treatment without laboratory testing.

Most patients with a suspected Lyme disease case require a stepwise serological assay to confirm the diagnosis. A positive preliminary enzyme immunoassay of patient serum is followed by a western immunoblot of IgM and IgG targeting Bb whole-cell lysate or a second enzyme immunoassay. Serology based assays for early acute Lyme disease have a sensitivity of ˜50%. In addition, the nature of serodiagnosis requires the host to mount an immune response over 1-2 weeks. This serological ‘Window Period’ increases the chance for the Bb infection to disseminate into the host before a clear clinical diagnosis is rendered. Moreover, enzyme immunoassay and immunoblots cannot differentiate between antibodies responding to an active infection and a previously treated infection. Thus, once seroconversion has occurred, reinfection with Lyme disease proves difficult to diagnose with the current testing protocol in the absence of an EM.

Ixodes spp. ticks can harbor additional pathogens such as Babesia microti simultaneously which can make diagnosis of Lyme disease more challenging. Studies indicate that 66% of residents of Long Island, New York diagnosed with Lyme disease were also seropositive against B. microti. Withstanding the challenges presented, novel Lyme disease detection methods are under development. Strategies include metabolomics, interferon-release assays, chemokine assays, xenodiagnositics, and proteomic markers.

### Example 1-4: Treatment

With a diagnosis in hand, antibiotics are effective for treating most Lyme disease infections. The majority of patients experience a resolution of symptoms after twice-daily doses of oral doxycycline, amoxicillin, or ceftriaxone for 2-4 weeks. Intravenous delivery of ceftriaxone for 30 days is required if there is evidence of CNS dissemination. In addition, a single prophylactic dose of doxycycline can prevent Lyme disease development if administered after the discovery of an Ixodes tick feeding for >24 hours in Lyme disease-endemic locations. In 7-30% of Lyme disease patients, antibiotic treatment leads to a transient Jarisch-Herxheimer reaction (JHR) with marked signs of inflammation and elevated C-Reactive Protein (CRP) during the first 10 days of treatment.

### Example 1-5: Post-Treatment Lyme Disease Syndrome

Chronic fatigue and musculoskeletal symptoms have been reported after the conclusion of antibiotic therapy treating Lyme disease. These patients are characterized within the broad and controversial category of Post-Treatment Lyme Disease Syndrome (PTLDS). Patients with PTLDS and a recurrent EM have been diagnosed with a re-infection from a second tick-bite via Bb genomic polymorphism sequencing. For other cases of PTLDS lacking an EM, further doses of antibiotics are not effective in relieving symptoms.

Prominent theories attempting to explain the discrepancy in Lyme disease antibiotic-refractory symptoms include: Bb OspA inducing cross-reactive autoimmunity towards the HLA-DRB1*0401 allele, components of Bb persisting near cartilaginous tissue leading to persistent inflammation, or a low level of persistent infection post-antibiotic treatment as indicated in murine and primate Lyme disease models.

In a recent case report, a biofilm has been histologically associated with Bb in human heart, kidney, liver and brain. This Bb biofilm was surrounded by infiltrating lymphocytes and stained positively for the specific biofilm marker alginate. The subject of the case report suffered from pericarditis, decreased cognitive function, and lymphocytic pleocytosis among other complications of disseminated Lyme disease. The 39-year-old subject's infection was refractory to multiple rounds of IV antibiotics. A Bb biofilm could explain the persistence of symptoms in the patient's clinical history. Another case report has also demonstrated intact Bb staining on CNS autopsy histology.

In a small cohort of PTLDS patients an increase non-specific antibody response directed against neural antigens. In a larger cohort of patients, PTLDS patients were found to have a statistically significant increase in the antibodies reactive to OspA (P31) compared to patients that remained symptom free after completing antibiotic therapy.

The current two-step diagnostic protocol cannot discriminate between a previous vs a new or refractory Lyme disease infection. This makes the characterization and treatment of PTLDS challenging. A novel diagnostic tracking the resolution of disease would better characterize a PTLDS patient's response to treatment and shed new light on this divisive topic.

### Example 1-6: a Crash-Course on Glycans

Glycans are linked monosaccharides conjugated to proteins and lipids in a stepwise post-translational fashion within the ER and Golgi apparatus. Glycans decorate over 70% of human proteins. Asparagine N-linked and serine/threonine O-linked glycans are small molecules that can be harvested from their glycoconjugates and characterized via high-performance liquid chromatography (HPLC) and mass spectroscopy (MS). Glycans comprise a diverse range of configurations and can undergo further chemical modifications such as acetylation or sulfation. In health, glycans participate in biochemical processes such as cell communication, cell-cell interactions, cell recognition, and fertilization. During disease, changes in glycosylation can drive metastatic properties, promote autoimmunity, and alter the effector function of antibodies.

Large scale sera glycome screening combined with glycoproteomics and novel analytical technologies allow for identification of disease-associated biomarkers. For example, the detection of altered core 1,3-fucosylation of alpha-1-anti-trypsin serves as a diagnostic for early forms of hepatocellular carcinomas.

### Examples 1-7: Diagnostics Under Development—where Glycans can Help

Direct study of Lyme disease's effect on the human sera glycome has not been accomplished. Therefore, the following genomic, proteomic, and metabolomic studies offer support for the application of glycomics and glycoproteomic research to Lyme disease.

A longitudinal transcriptome analysis of 29 Lyme disease patients revealed specific changes in gene expression during the first 3 weeks of infection. Four of the six independently verified proteins altered during early Lyme disease [C9, CRP, CST6, PGLYRP2] are N-glycosylated. In a separate study, acute phase markers measured from the serum of untreated acute Lyme disease patients revealed elevated levels of CRP, Serum amyloid A, and T-cell specific mediators which correlated to elevated liver enzymes. Yet another group identified acute phase proteins CRP, complement component 9, and trypsin inhibitor heavy chain 2, from human sera via MStern blotting-based serum proteomics. The increased levels of Interleukin-1 receptor accessory protein, Serum amyloid A-1, and Serum amyloid A-2 were found in disseminated Lyme disease sera which proved to be predictive in quantifying the stage infection. Furthermore, a multi-omic analysis of the murine macrophage response to Bb led to the downregulation of the glycosylated CD180 protein and an altered metabolic phenotype. All the proteins identified should be analyzed to determine if their associated N-glycan structures are modified in a specific manner in response to the Bb infection. For example, CRP is differentially induced as a glycosylated molecular variant in response to specific disease states such as Tuberculosis. It was expected that Bb would induce a specific CRP glycosylation pattern during early disease.

Akin to changes in protein levels, acute Lyme disease alters eicosanoid, bile acid, sphingolipid, and carnitine host metabolic pathways as indicated from human sera and urine. Specific metabolite signatures were also identified comparing PTLDS vs non-PTLDS sera. Glycan synthesis pathways respond to altered metabolic and/or inflammatory states. Therefore, the perturbation of host cellular metabolism supports the rationale for the detection of altered glycans in early Lyme disease, disseminated Lyme disease, or PTLDS.

Bb itself is also no stranger to the world of human glycans. For example, patients deficient in Mannose-Binding Lectin (MBL) complement pathway were at higher risk to develop disseminated Lyme disease. Lectins recognize specific glycan groups. Thus, a defective MBL complement pathway not clearing the Bb infection highlights just one of the roles that glycans play during Lyme disease.

Many of the Bb adhesins target N-glycosylated extracellular proteins including decorin, aggrecan, integrin, and plasminogen. Lyme disease arthritis severity was increased in a murine model deficient in the lysosomal beta-glucuronidase responsible for clearing glycosaminoglycans (GAGs). Therefore, a Bb infection appears to promote the turnover of glycosylated products in the synovium during disseminated disease. Transcriptomic analysis also revealed increased glycosaminoglycan/proteoglycan-biding activity by Bb during CNS tissue colonization in a primate model. It was expected that the buildup of these glycosylated proinflammatory GAGs in synovium can be detected in human sera and be utilized as a marker of disseminated Lyme disease. It was also expected that a cellular response to the binding of Bb adhesins to extracellular proteins would lead to an alteration in host-glycosylation during infection.

### Glycan Immunoglobulin Modulation Applications

In addition to glycans serving as acute-phase biomarkers, studies have implicated the alteration of glycans on Immunoglobulin Fc regions to the modulation of immune responses in cancer and autoimmune disorders. Examples of glycosylation of immunoglobulins promoting aberrant disease include Rheumatoid Arthritis via IgG, IgA Nephropathy, and IgE-induced anaphylaxis. Myriad of factors can affect B-cell glycosyltransferase activity during antibody production; ‘fine-tuning’ the immune-cell receptor downstream signaling towards either a pro- or anti-inflammatory pathway.

Thus, in contrast to the first goal of early detection, the glycosylation pattern on IgG or IgM may allow clinicians to track their patient's response to Lyme disease treatment. PTLDS patients would also gain insight into the source of their symptoms via analysis of their immunoglobin glycosylation patterns.

### Example 1-8

Glycomic analysis of human sera brings fresh potential to Lyme disease diagnostics at the acute infection and post-treatment stages. The analytic technologies required to interrogate glycans in human sera have been validated in cancer and autoimmune disease. Proteomic studies of Lyme disease have identified N-glycosylated targets in acute Lyme disease sera. Lastly, analysis of the Fc portions of IgM and IgG glycans may have the potential to track immune response during treatment or disease reactivation in PTLDS.

### Example 2: Glycoproteomic Approach to Lyme Disease Diagnostics

In Example 2, evidence that altered glycosylation of immunoglobulin G and M (IgG, IgM) has great potential as a diagnostic and prognostic biomarker for Lyme disease (LD) will be presented. The technological basis of the approach is glycomics coupled with liquid chromatography and electrospray ionization mass spectrometry (LC-ESI-MS).

This diagnostic test uses less than 50 μL of patient serum. In general, in this test, total serum immunoglobulins are purified, and the N-linked sugars are enzymatically released, labeled, and identified. Glycan moieties that change with LD are quantitated (UPLC-ESI-MS) and subjected to an algorithm trained on previous data sets. A positive result indicates an active acute LD infection. In addition, the test can detect patients who responded to antibiotic therapy. In response to our survey of patient and clinician needs, we address the current diagnostic deficiencies for acute LD with our diagnostic.

Advantages of the glycan-based diagnostic include, but are not limited to: (i) detect acute LD biomarkers before seroconversion, (ii) accurately confirm an active case of acute LD in patients with a history of previous LD infections, (iii) track therapeutic outcomes, (iv) discriminate acute LD from healthy endemic controls, late-stage LD, and other ‘mimic’ inflammatory diseases, and (v) detect acute LD with higher sensitivity than current two-tiered diagnostics. Example 2 presents data for each stated claim.

Advantages of the technology include but are not limited to: (i) adapts existing clinical mass spectrometry instruments to run high-throughput Lyme disease diagnostics, (ii) assays require small volumes of serum, and (iii) the assay produces highly reproducible results.

To the best of the inventors' knowledge, GlycoLyme is the first approach that uses glycomics coupled with LC-ESI-MS to diagnose acute LD. Because ESI-MS is considered a routine diagnostic tool in clinical laboratories, the herein test could quickly be adapted to high-throughput workflows. Glycomics is expanding the possibilities of diagnostics in multiple fields. Because glycosylation is a dynamic process it can be used to monitor disease progression and personalize medicinal treatments.

### Example 2-1: Description of Solution

Direct testing for LD is elusive because the spirochete has a limited presence in the bloodstream. Thus, testing has historically relied on indirect methods of detection, specifically Borrelia burgdorferi (Bb)-reactive antibodies.

The present study observed that symptoms of acute LD include swollen lymph nodes, indicating that the lymphatic system is disrupted early in the disease progression before seroconversion occurs. The glycan-based assay detects the lymphatic system's response to the disseminating spirochete's modulation of the immune system. The present study studied this immuno-modulation by assaying the N-glycome of circulating IgG and IgM. It is worth noting that the test herein does not rely on detecting seroconversion or Borrelia-specific IgG or IgM. Instead, the test analyzes the global or total immunoglobulin glycosylation profile.

IgG and IgM serum glycoproteins are produced by B cells in the lymph nodes and play an important role in the LD immune response. Both immunoglobulins contain tightly regulated N-linked glycans. Moreover, antibody glycosylation affects antibody function and can reflect acute and convalescent disease states.

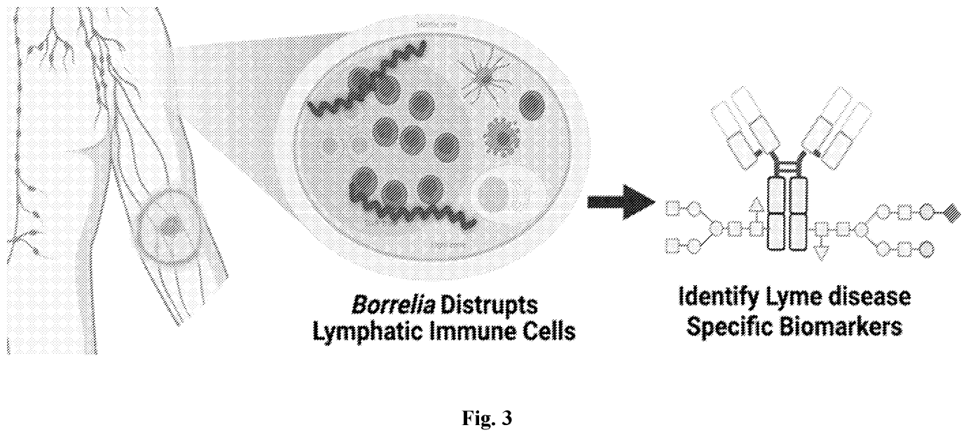

Because Bb invades the lymphatic system during acute LD and ultimately disrupts the antibody-producing germinal centers in the lymph tissue, it was hypothesized that the invasion of the lymphatic system by Borrelia would have a global impact on the glycosylation status of IgG and IgM (FIG. 3). This approach is based on the B cell perturbance and circumvents the need to wait for patient's Bb-specific seroconversion.

During infection, the pathogen induces transcription factors that regulate cellular glycosyltransferases, resulting in altered glycan moieties. Small glycans occupy IgG during most inflammatory conditions to promote immune activation by lowering steric hindrance to binding downstream cellular receptors. Inflammatory diseases normally show a trend for global IgG N-glycans to reduce galactose and sialic acid content. Surprisingly, acute LD IgG N-glycans increase their galactose and sialic acid content. Accordingly, the present glycan-based approach diagnoses acute disease while also identifying immuno-modulation that reduces the host's ability to clear a Borrelia infection.

### Example 2-2: Initial Validation Data

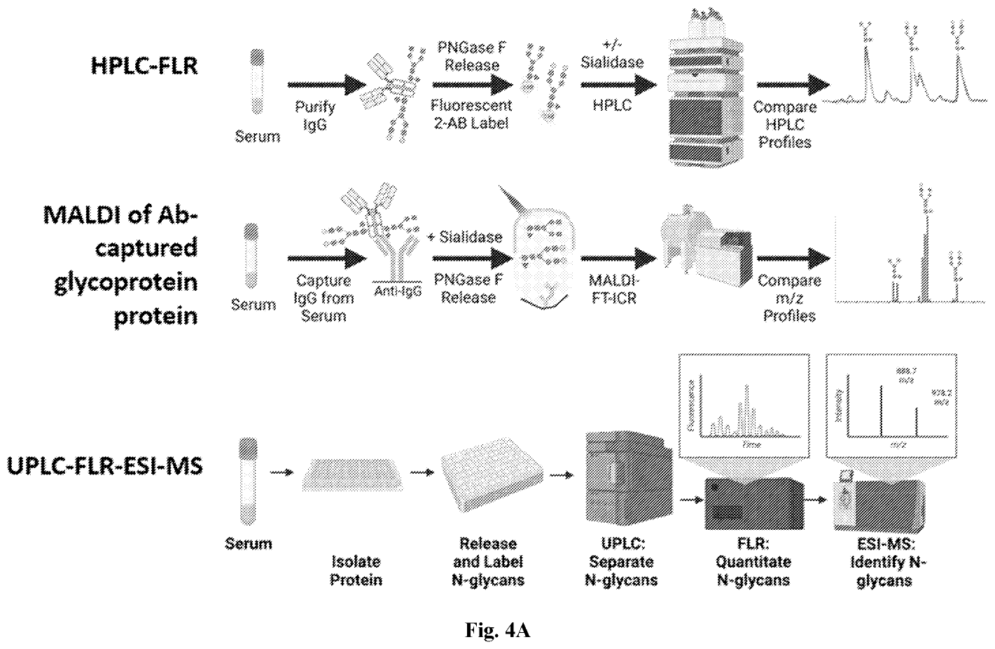

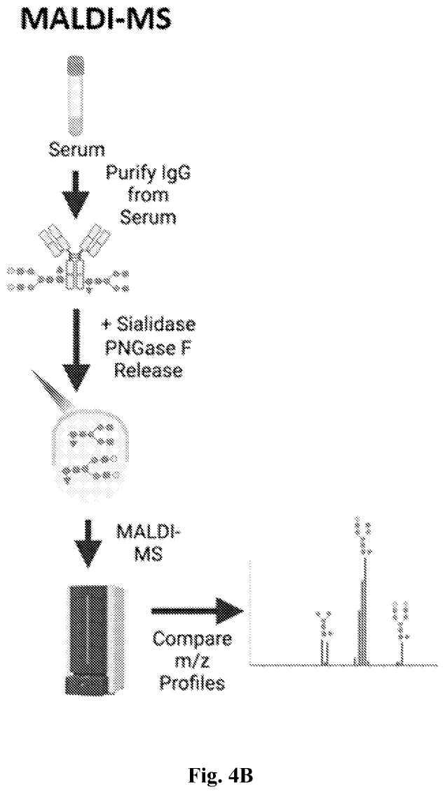

Patient sample sets were curated by The Bay Area Lyme Foundation and the CDC Lyme disease serum repository Panel 1. The data was generated by the Comunale Lab at Drexel University College of Medicine using serum samples supplied by The Bay Area Lyme Disease Biobank, the Center for Disease Control, and Precision for Medicine Group LLC. The analysis was completed using UPLC-ESI-MS and confirmed using other glycan sequencing and quantification methods (FIGS. 4A-4B). Each glycan was quantitated by calculating the peak area as a percent of the total in the chromatography methods and by peak height in mass spectrometry methods.

N-glycans are complex sugar structures that are covalently linked to many circulating proteins. Depending on the state of the immune system, different N-glycans species occupy specific sites on immunoglobulins. These N-glycans impact protein function due to the steric hindrance (varied size and charge) for protein binding partners. For example, IgG Fc tails with bulky, negatively charged N-glycans is blocked from binding lymphocyte receptors to signal for inflammation. Only one of 24 glycans can occupy the single glycosylation site on the heavy chain of IgG and one of 6 mannose glycans or 24 complex glycans can occupy the five IgM glycosylation sites. The high degree of N-glycan variability during immune responses allows for multiple biomarkers to identify and track disease states. To analyze glycoprotein N-glycan composition, the N-glycans were released from purified proteins using an enzyme and separate the N-glycans by size using chromatography.

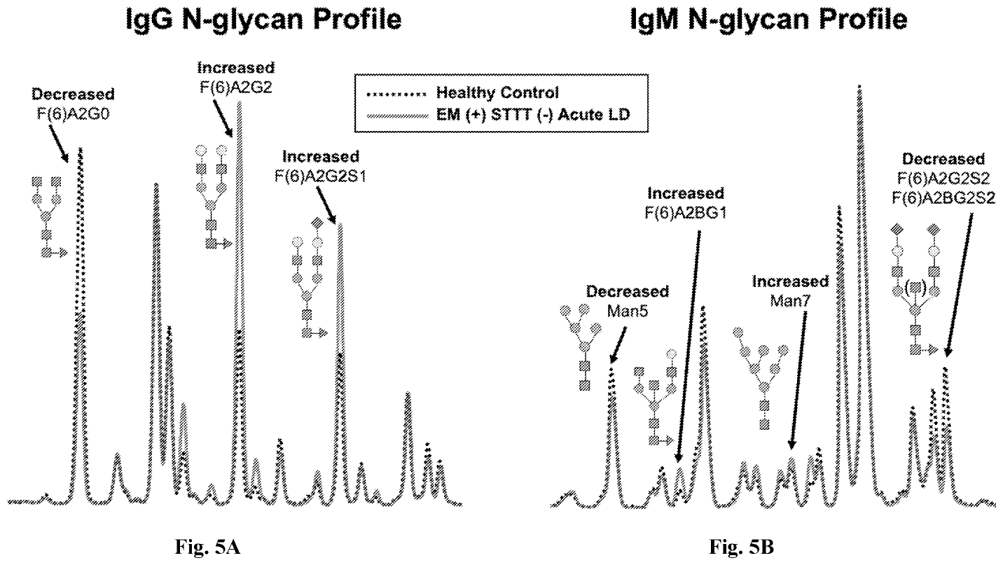

It was demonstrated that the glycan-based assay can detect acute Lyme disease biomarkers before seroconversion. Representative chromatograms of N-glycans released from total IgG and IgM from patients with acute Lyme disease and healthy controls are presented in FIGS. 5A-5B. Arrows label multiple N-glycans that specifically increase or decrease in abundance during acute Lyme disease from the CDC Research Panel 1. IgG contains significantly more galactose and sialic acid (FIG. 5A) while IgM increases the length of the mannose N-glycans and loses di-sialylated N-glycan structures (FIG. 5B). These N-glycan profiles are not canonically associated with inflammatory disease responses and suggest immuno-modulation occurs as the Borrelia spirochete interacts with the lymphatic system. By combining the observed abundance shifts in the N-glycan profiles of IgG and IgM, we propose that N-glycans represent an early biomarker of immuno-modulation in response to acute Lyme disease

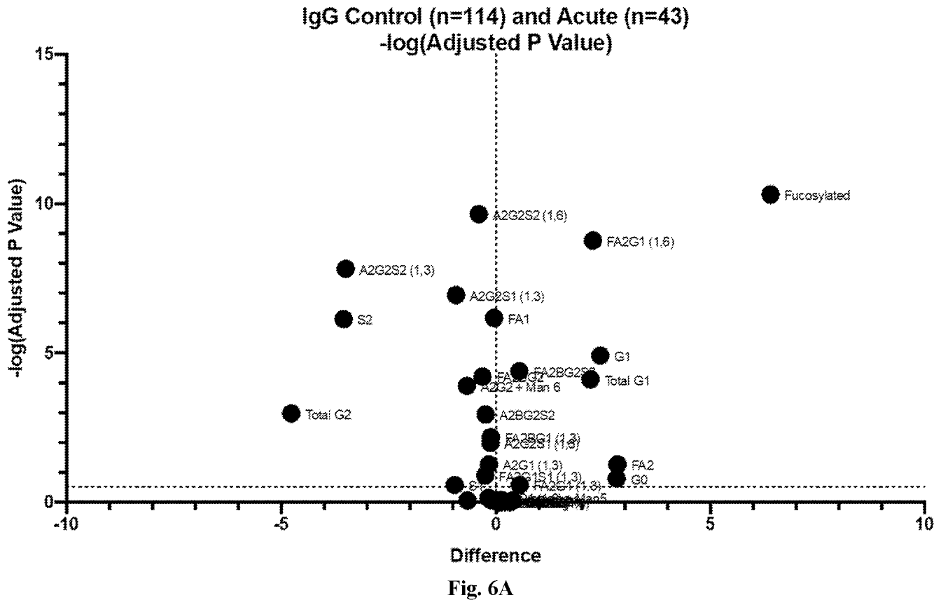

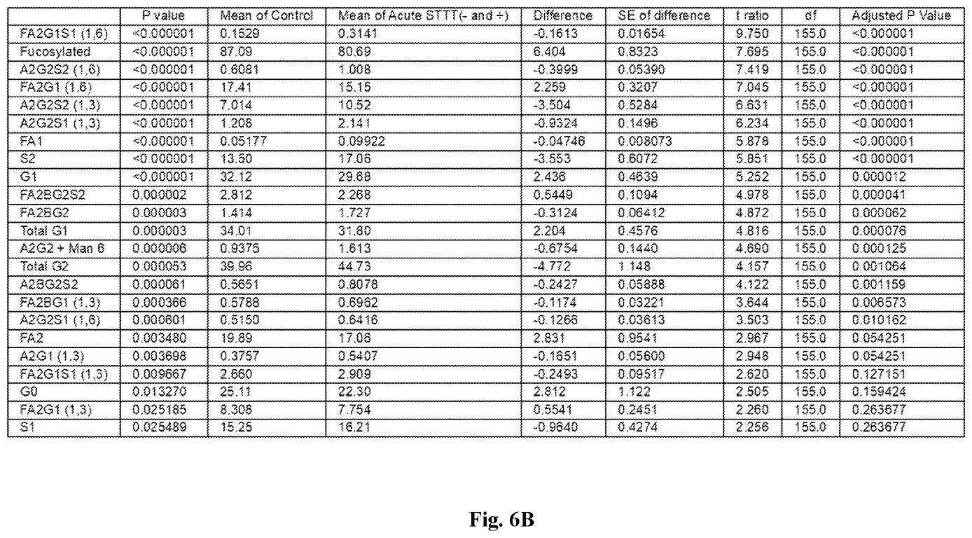

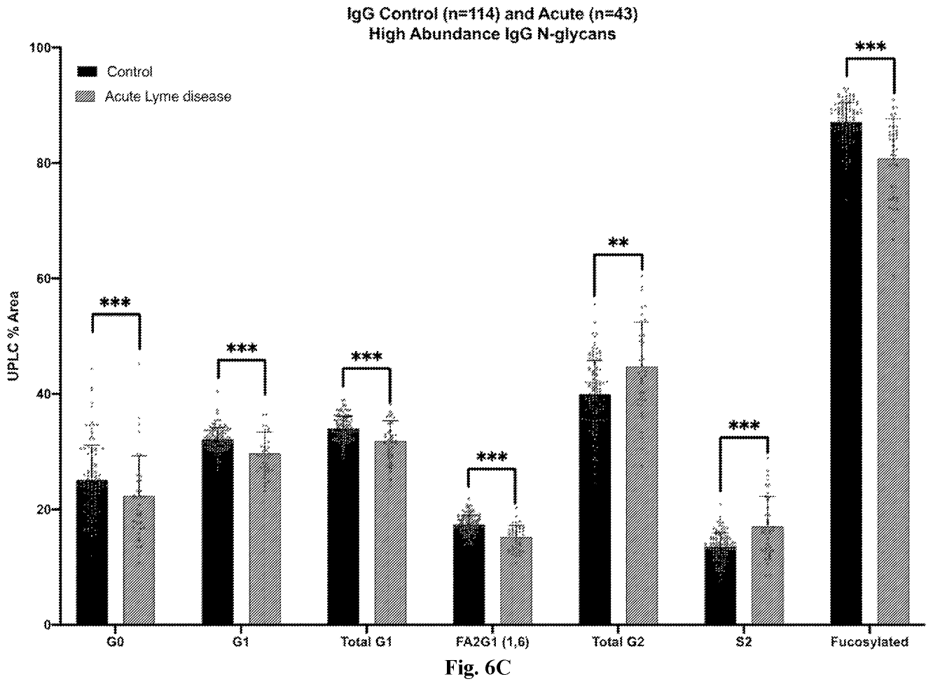

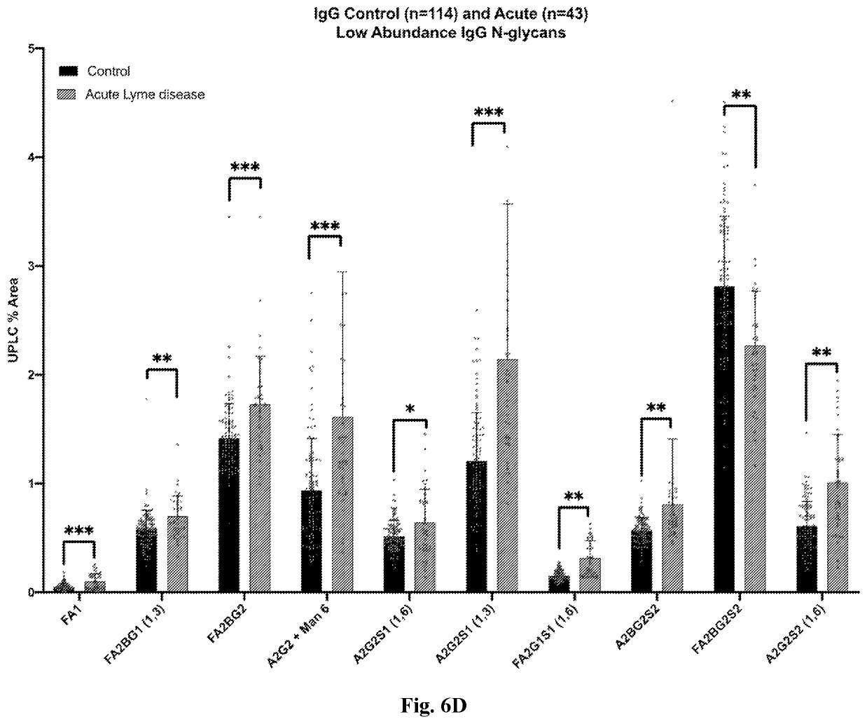

It was demonstrated that IgG glycosylation can accurately differentiate acute LD from healthy controls. IgG N-glycosylation Volcano plot identified N-glycans from UPLC-FLR-ESI-MS that differ significantly during acute Lyme disease (n=43) compared to healthy controls (n=114). The top 17 statistically significant N-glycans differing during acute Lyme disease are plotted in the graphs to the right, separated by high- and low-abundance (FIGS. 6C and 6D). IgG N-glycans classes: G0 (agalactosylated), G1 (monogalactosylated), and Fucosylated decrease. Conversely the G2 (di-galactosylated), S1 (monosialyated), and S2 (Di-sialyated)N-glycans increase in abundance. Data presented as means+/−standard deviation with statistical significance denoted from multiple t-tests by *p>0.05, **p>0.01, ***p>0.001.

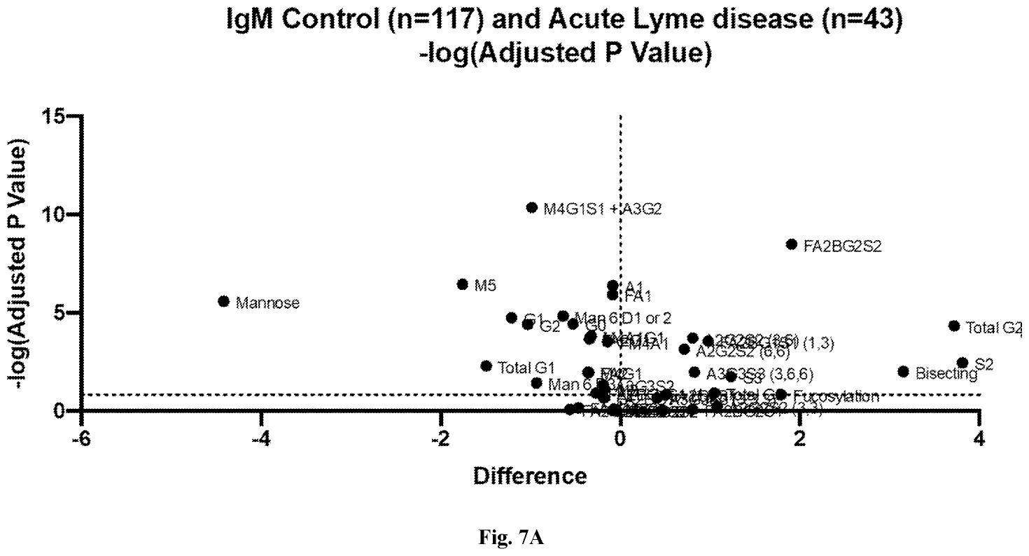

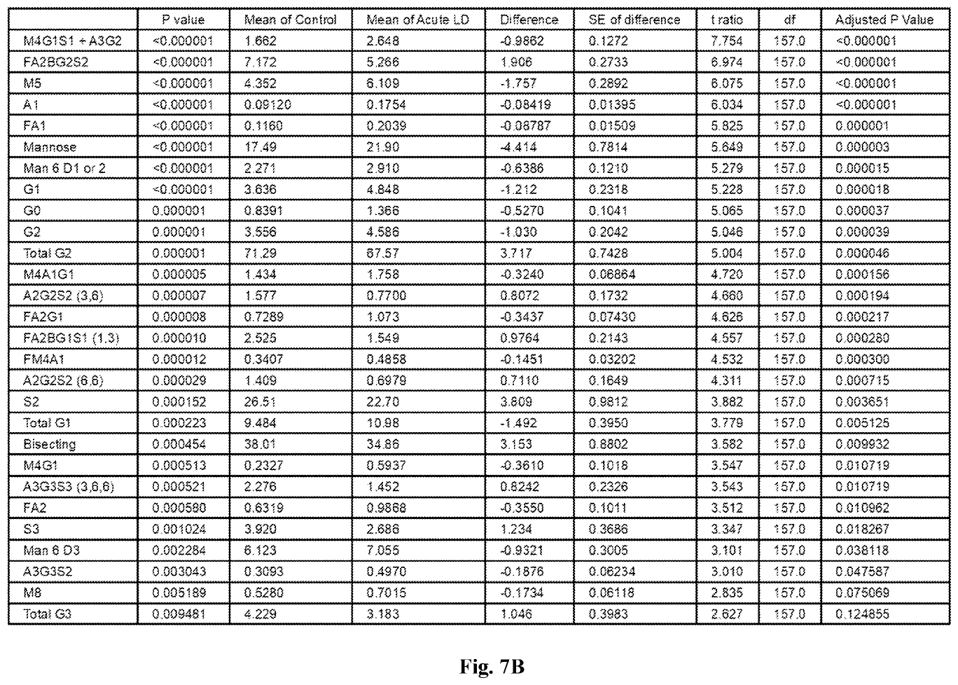

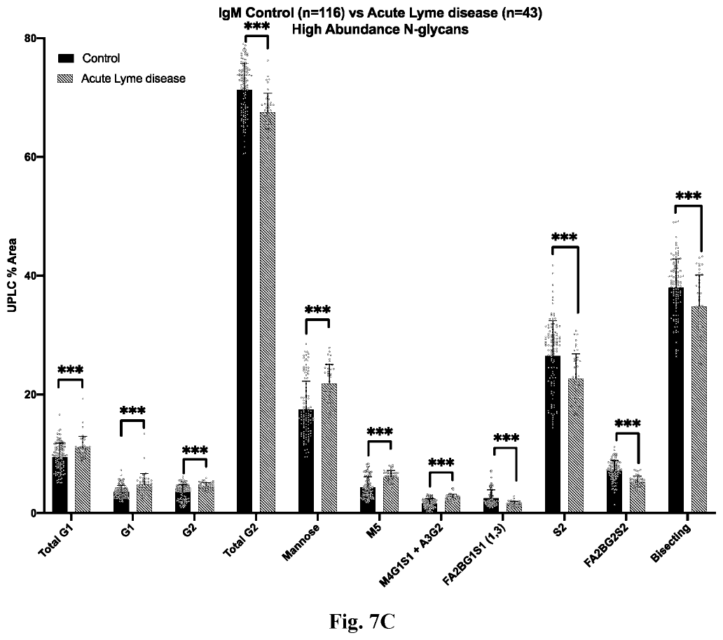

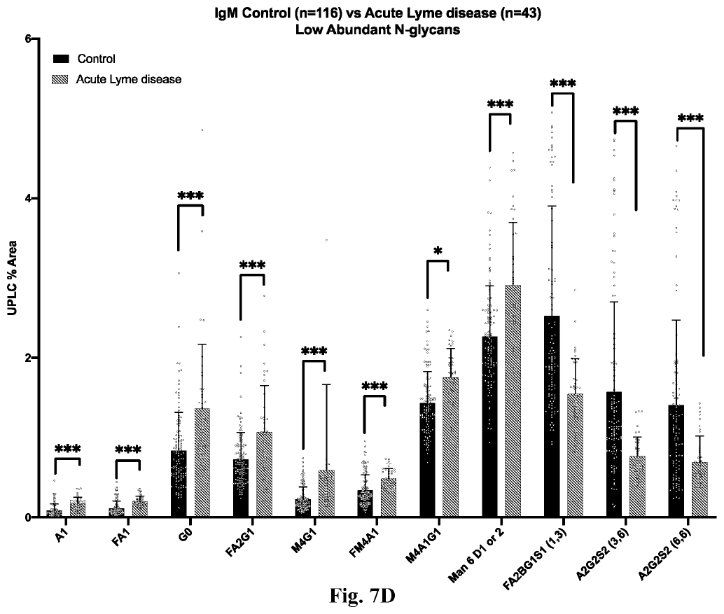

It was demonstrated that IgM glycosylation can accurately differentiate acute LD from healthy controls. IgM N-glycosylation Volcano plot identified N-glycans from UPLC-FLR-ESI-MS that differ significantly during acute Lyme disease (n=43) compared to healthy controls (n=116) (FIGS. 7A-7B). The top 21 statistically significant N-glycans differing during acute Lyme disease are plotted in the graphs to the right, separated by high- and low-abundance (FIGS. 7C and 7D). For IgM, the S1 (mono-sialyated), S2 (di-sialyated) and Bisecting classes decrease. Conversely the G1 (monogalactosylated), G2 (di-galactosylated), and Mannosylated N-glycans increase in abundance. Data presented as means+/−standard deviation with statistical significance denoted from multiple t-tests by *p>0.05, **p>0.01, ***p>0.001.

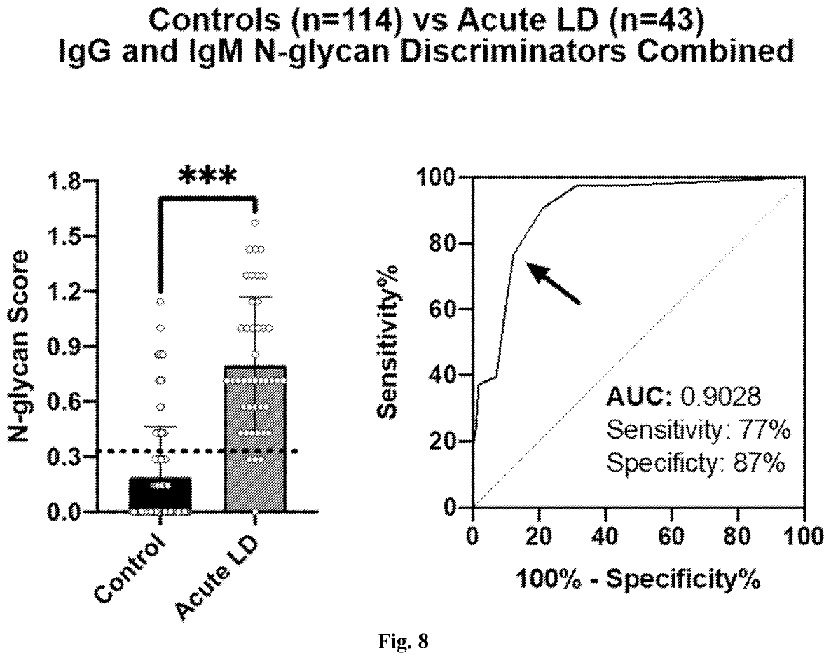

It was demonstrated that when IgG and IgM glycosylation was combined in one algorithm, the test was able to separate a majority of the acute Lyme disease cohort from healthy controls (FIG. 8). The resulting ROC curve is presented with an area under the curve (AUC) of 0.9028, sensitivity of 77%, and specificity of 87%.

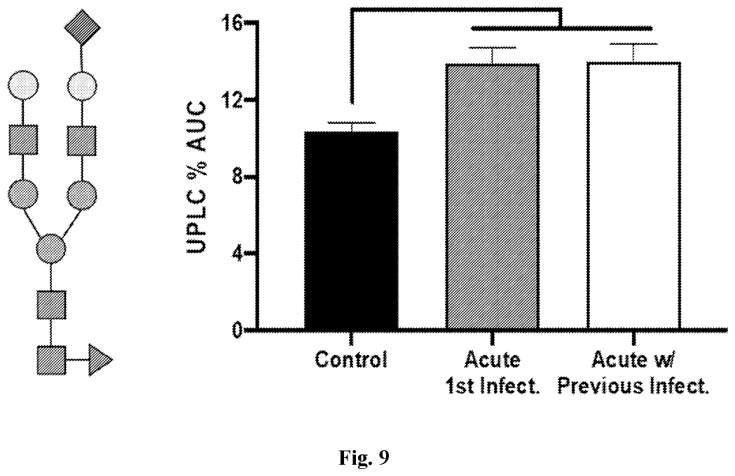

It was demonstrated that the glycan-based assay can accurately confirm acute LD in patients with a history of previous LD infections. FIG. 9 exemplifies that the changes in IgG N-glycan content in acute Lyme disease patients with a primary infection or those that have been reinfected. Both are associated with active disease. There are no statistically significant differences between acute LD patients infected for the first time (n=9 Acute 1^{st} Infect.) compared to those who were infected with LD previously (n=9 Acute w/Previous Infect.). Therefore, the biomarkers for immuno-modulation observed during primary acute infection can be detected each time a patient is re-infected with acute LD. This is in direct contrast to the current STTT testing paradigm wherein it is unclear if a patient with a previous history of LD has already seroconverted or is responding to the reinfection. As such, the test herein can add clinical value and help clinicians treat patients faster with confidence independent of LD infection history.

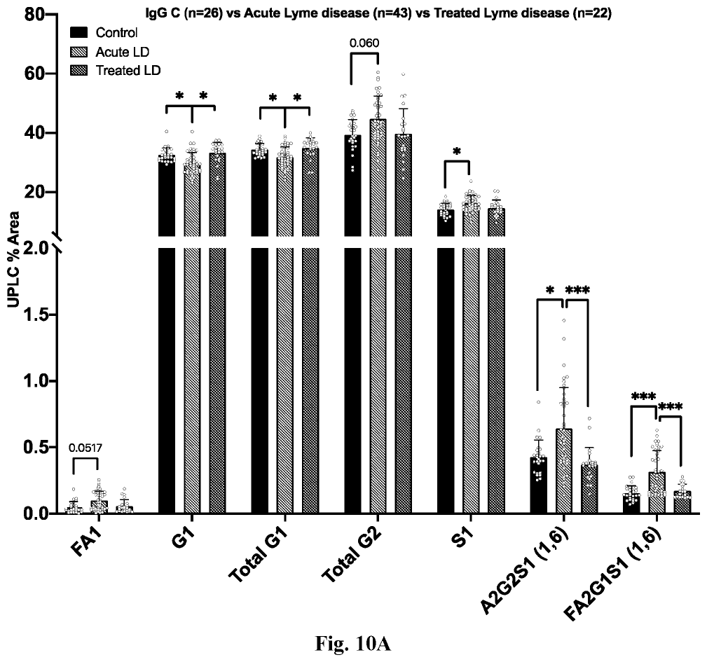

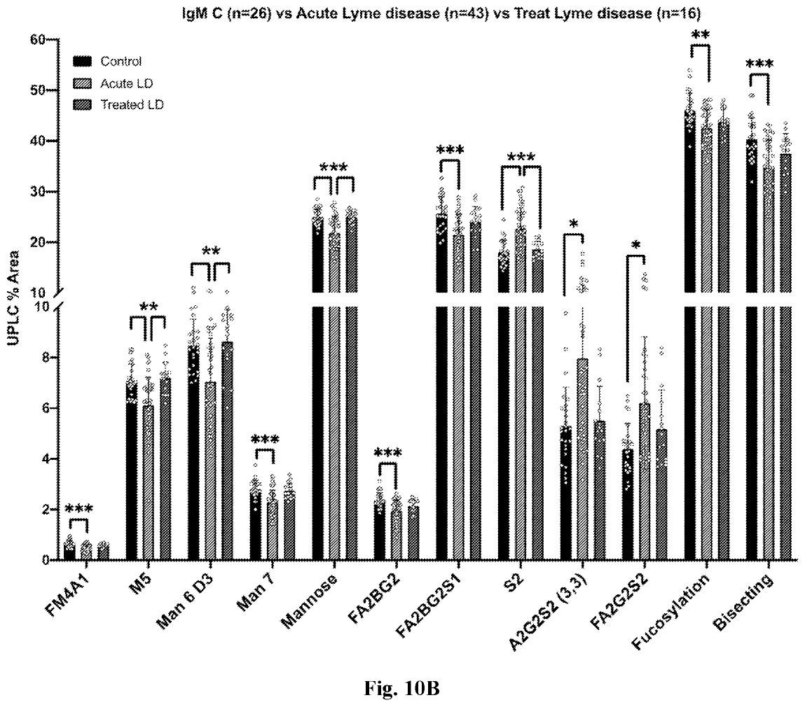

The present study demonstrated that the glycan-based assay herein can track therapeutic outcomes. Patients with acute LD (n=18) returned to donate a serum after completing 10-21 days of antibiotic treatment and 70-90 days of convalescence. None of the 18 patients returning to the clinic reported symptoms that suggested they had Post-treatment Lyme disease Syndrome (PTLDS). FIGS. 10A-10B identify the progression to a healthy control glycosylation profile for IgG (FIG. 10A) and IgM (FIG. 10B). The most notable IgG trend to normalcy is shown by the decrease in sialic acid content. For IgM, the increase in mannose structures resembling a healthier profile is prominent, along with the return of increased bisecting structures, while the sialylated structures revert to lower levels.

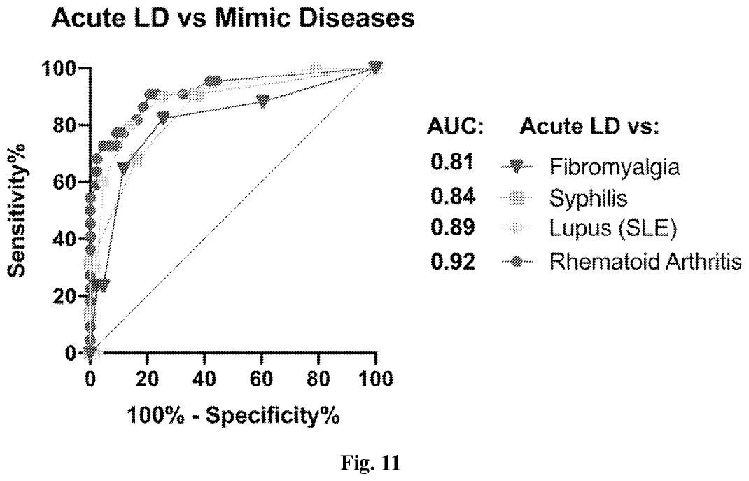

FIG. 11 demonstrates that the glycan-based assay can discriminate acute LD from and other diseases whose clinical manifestations may present similarly to Lyme disease, hence are known as Lyme ‘mimic’ diseases. Patients' N-glycan profiles from total IgG and IgM were scored using set thresholds for each discriminating N-glycan. Acute Lyme disease can be discriminated from the mimic diseases with high accuracy as evidenced by the AUCs range of 0.81-0.92.

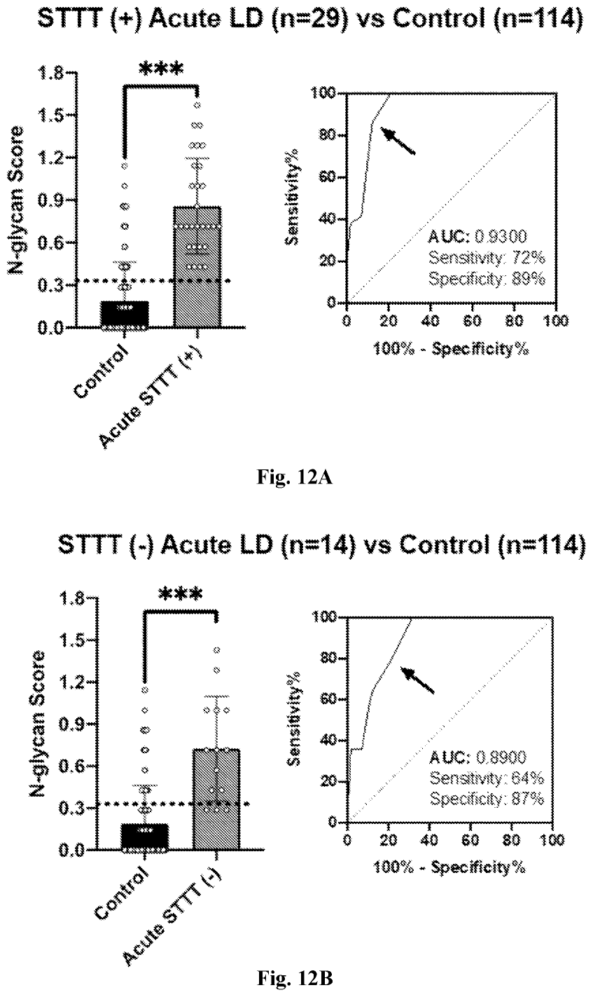

In the current study, the GlycoLyme test was able to discriminate between healthy control samples and patients with STTT (+) and STTT (−) acute Lyme disease with greater accuracy than that of the current FDA cleared Lyme disease diagnostics. IgG and IgM N-glycan scores discriminate EM (+) STTT (+) patients with 72% sensitivity and 89% specificity and an AUC of 0.93 (FIG. 12A). IgG and IgM N-glycan scores discriminate EM (+) STTT (−) patients with 64% sensitivity and 87% specificity and an AUC of 0.89 (FIG. 12B). In comparison, the sensitivity of the GlycoLyme test is higher than the FDA cleared conventional two-tiered testing for acute Lyme disease sensitivity is ˜₄₆%. Hence, by combining total IgG and IgM N-glycan profiles, the present study discriminated between endemic healthy control and acute LD serum from patients that seroconverted and, importantly, from patients that have not yet seroconverted. Therefore, the glycan-based assay herein can significantly improve the number of patients that are diagnosed with acute Lyme disease. Specific patient populations that would benefit from the glycan-based assays are those that previously tested positive for LD who have become re-infected, patients with a skin pigmentation that obscures the classic EM rash, and those patients who wish to track their response to treatment over time.

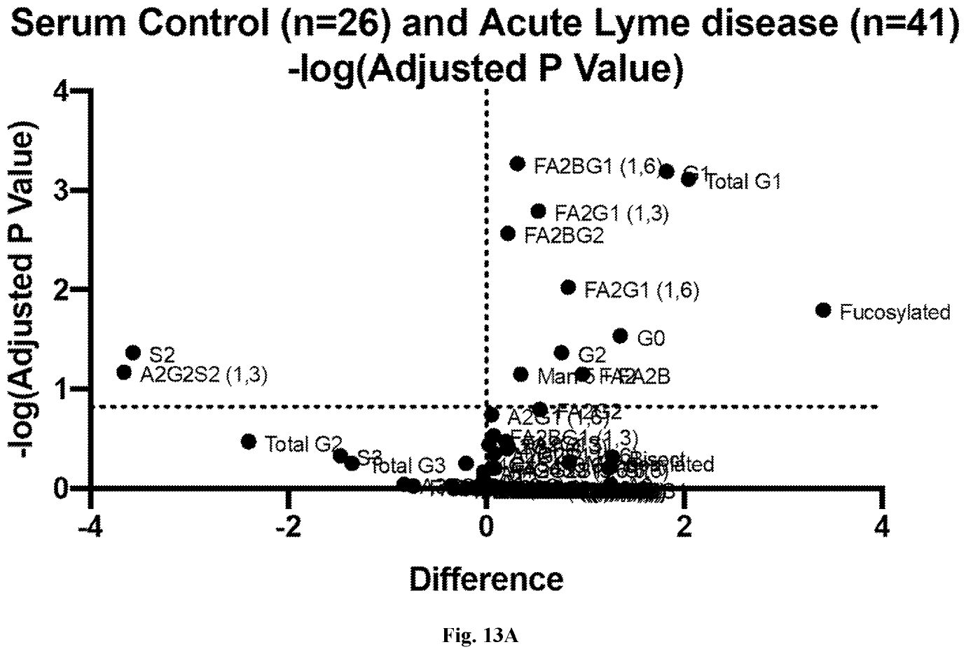

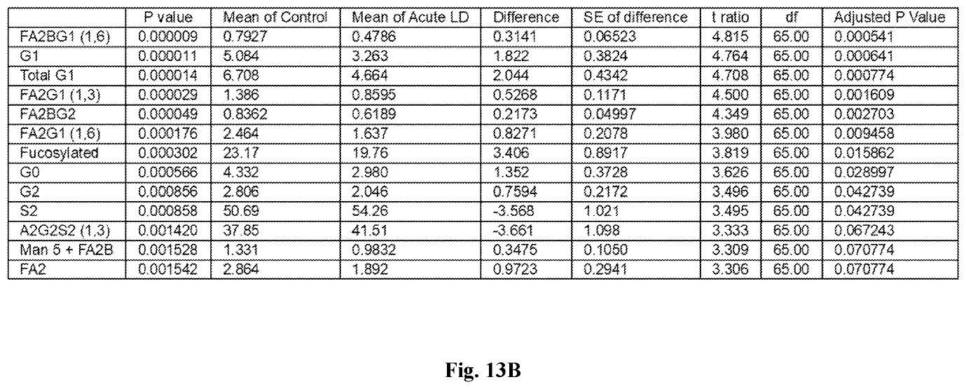

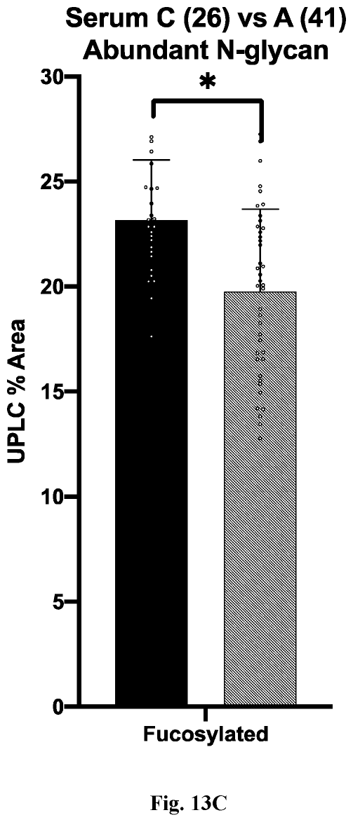

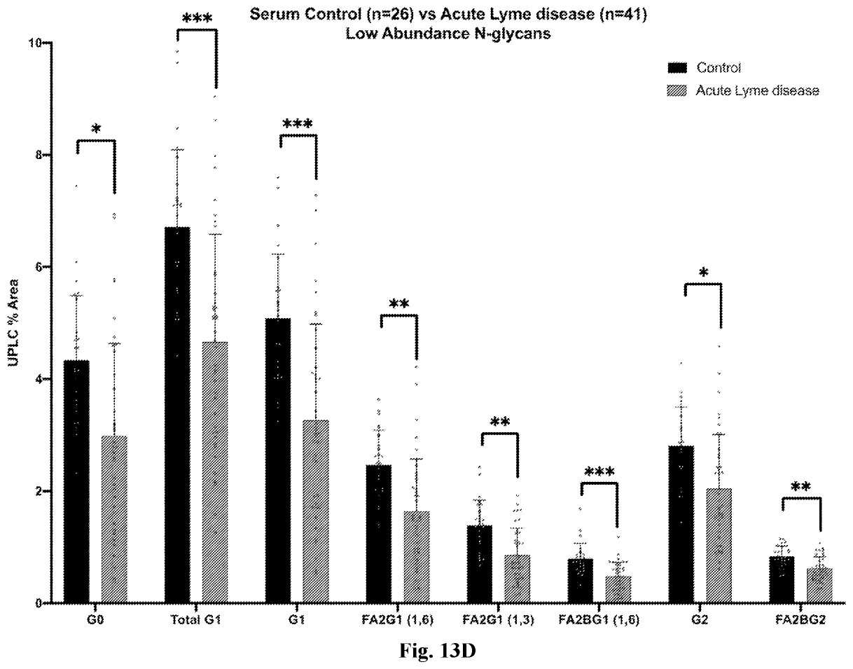

In the current study, the GlycoLyme test was able to discriminate between healthy and acutely infected Lyme disease patients using the total serum glycome analysis. The top 9 statistically significant N-glycans differing during acute Lyme disease are plotted in graphs (FIGS. 13A-13D), separated by high- and low-abundance. For total serum N-glycans, the fucosylated, G0 (agalactosylated), G1 (mono-galactosylated), and G2 (di-galactosylated) N-glycans decreased compared to healthy controls. Data presented as means+/−standard deviation with statistical significance denoted from multiple t-tests by *p>0.05, **p>0.01, ***p>0.001.

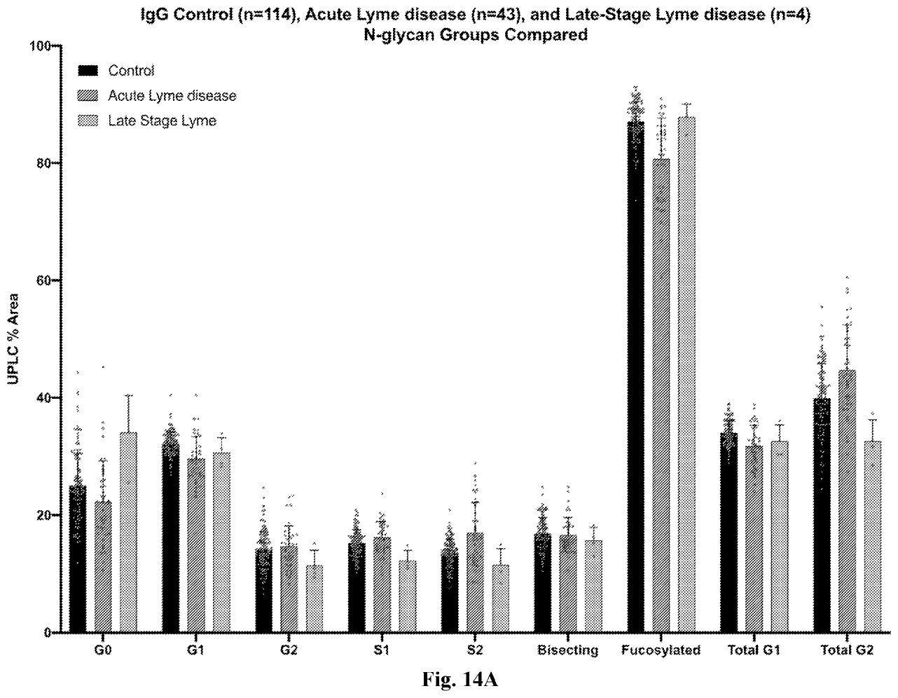

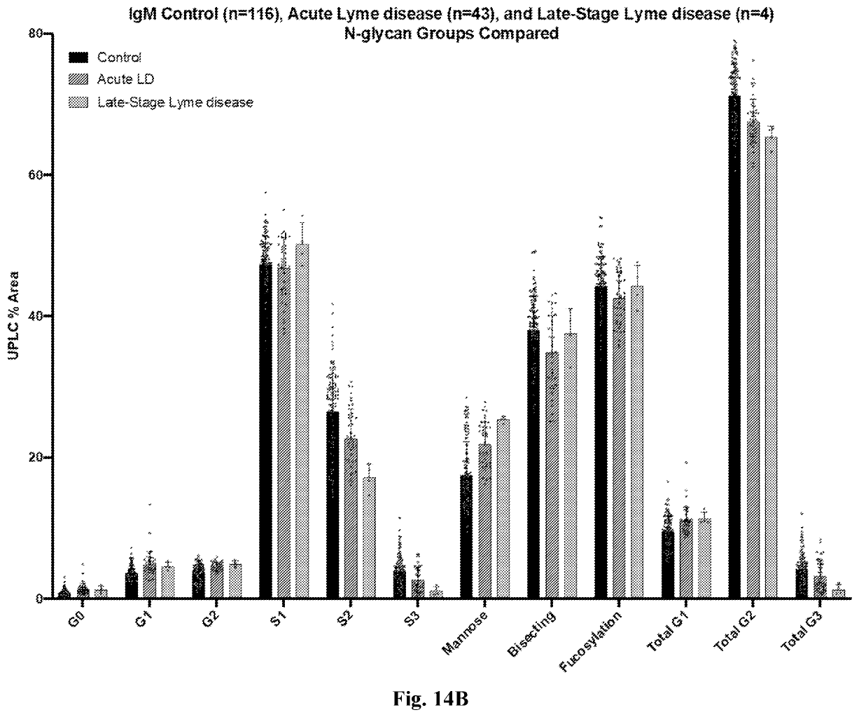

In the current study, the GlycoLyme test was able to discriminate between acute Lyme disease and late-stage Lyme disease (FIGS. 14A-14B). Comparison of IgG and IgM N-glycans from healthy controls, acute Lyme disease, and an n=4 of late-stage Lyme disease (2 Lyme arthritis patients and 2 Lyme Neuroborreliosis patients) (FIG. 14A). During acute Lyme disease, IgG the G0 N-glycans decrease while in late-stage Lyme disease the G0 increases to levels higher than healthy controls indicating a significant change in disease response occurring during late-stage Lyme disease. Moreover, the larger G2, S1, and S2 N-glycans increase during acute Lyme disease while decreasing lower than healthy control levels in late-stage Lyme patients. FIG. 14B. IgM N-glycans S1 and Mannose increase during late-stage Lyme compared to acute Lyme disease while the S2, S3 and total G3 classes of N-glycans decreased in late-Lyme disease compared to acute Lyme disease. Data presented as means+/−standard deviation without statistical analysis due to the n=4 sample number for late-stage Lyme.

### Example 2-3: Use of GlycoLyme

### General Use:

The GlycoLyme test is intended as a qualitative measure of aberrant total IgG and IgM N-glycans in human serum with or without total serum N-linked glycosylation from symptomatic patients with suspected acute B. burgdorferi infection.

The GlycoLyme test can be prescribed by a clinician at the initial visit to confirm the diagnosis of acute LD and to ensure the patient has a baseline for monitoring treatment efficacy. Subsequent tests should be conducted to confirm treatment response or upon subsequent infections.

The GlycoLyme test is intended to be used in conjunction with antibody titers for suspected acute LD re-infections. The glycan profile will indicate an active infection and will be used to monitor treatment responses.

The assay is also intended to be used as a second-tier confirmation test following a positive first-tier enzyme immunoassay (EIA).

### Predicted Outcome of Use:

The test provides patient centeredness in that the patient will be able to have (i) confirmation that the treatment is working or (ii) a metric that will correlate their continued and unresolved symptoms with a test result. Subsequent tests are administered following treatment as needed and compared to the results of the initial visit.

The test provides the treating clinician with a test result that informs on antibiotic effectiveness and guide treatment decisions.

### Example 2-4

### Lyme Disease is on the Rise

With the rates of LD climbing, it is crucial to have accurate diagnostics that can detect an acute LD infection. In addition to the percent of ticks infected with Borrelia burgdorferi, climate change is causing an increase in the hospitable tick habitat and altered deer populations.

Undiagnosed LD Quickly Disseminates into the Host Organs, Making Treatment More Difficult

When acute LD goes untreated, the spirochete disseminates into synovial, cardiac, and neuronal tissue resulting in chronic disabilities and, in severe cases, death. Pregnant women who do not receive treatment have experienced multiple adverse pregnancy outcomes including, preterm delivery, stillbirth, and congenital cardiac malformations. In addition, children's hospitals report increased Lyme carditis in minors. These disseminated cases of Lyme disease would be prevented by an accurate diagnostic to catch the acute stage of disease, when a two-to-three-week course of antibiotics effectively treats patients. Due to the low sensitivity of the current acute Lyme disease diagnostic, patients with Lyme disease are often missed as “false negatives.”

### Early Diagnosis Means Lowered Disease Burden

The GlycoLyme test improves sensitivity and minimizes false negatives, significantly reducing disease burden. By lessoning the long-term cost of disseminated disease in disability-adjusted life years (number of years lost due to ill-health, disability, or early death) the endemic population will show measurable improvements in ‘healthy’ life lost by virtue of being in states of poor health.

### Example 3: Comparison of Glycosylation Profiles

The present study compared IgG, IgM and total serum N-glycan profiles of control subject, subjects with acute Lyme disease, subjects with rheumatoid arthritis, subjects with syphilis, subjects with lupus, and subjects with fibromyalgia. The results are listed in the Tables below:

TABLE 1
IgG N-glycan profiles, Control Vs. Acute Lyme Disease

| | Adjusted | Mean of | Mean of | |
|---|---|---|---|---|
| | P Value | Control | Acute LD | Difference |
| FA2G1S1 (1, 6) | 0.000252 | 0.1554 | 0.3141 | −0.1587 |
| G1 | 0.021261 | 32.58 | 29.68 | 2.895 |
| A2G2S1 (1, 6) | 0.03748 | 0.4269 | 0.6416 | −0.2147 |
| S1 | 0.037962 | 14.11 | 16.21 | −2.1 |
| Total G1 | 0.039972 | 34.33 | 31.8 | 2.523 |
| FA1 | 0.051714 | 0.04926 | 0.09922 | −0.04996 |
| Total G2 | 0.060205 | 39.3 | 44.73 | −5.424 |
| FA2G1 (1, 6) | 0.166737 | 16.53 | 15.15 | 1.38 |
| FA2G1 (1, 3) | 0.166737 | 8.866 | 7.754 | 1.112 |
| Fucosylated | 0.166737 | 84.95 | 80.69 | 4.255 |
| S2 | 0.190136 | 14.02 | 17.06 | −3.031 |
| A2G2S2 (1, 6) | 0.214032 | 0.7502 | 1.008 | −0.2578 |

Increases in the following IgG N-glycans over normal indicate acute Lyme disease: FA2G1S1 (1,6), A2G2S1 (1,6), S1, FA1, Total G2, S2 and A2G2S2 (1,6). Decreases in the following IgG N-glycans over normal indicate acute Lyme disease: G1, Total G1, FA2G1 (1,6), FA2G1 (1,3) and fucosylated.

TABLE 2
IgG N-glycan profiles, Acute Lyme
Disease Vs. Lyme Disease, Treated

| | Adjusted | Mean of | Mean of | |
|---|---|---|---|---|
| | P Value | Acute LD | Treated LD | Difference |
| FA2G1S1 (1, 6) | 0.005055 | 0.3141 | 0.1721 | 0.142 |
| FA2G1 (1, 6) | 0.005055 | 15.15 | 17.37 | −2.213 |
| A2G2S1 (1, 6) | 0.005961 | 0.6416 | 0.3694 | 0.2722 |
| G1 | 0.012415 | 29.68 | 33.25 | −3.566 |
| A2G2S2 (1, 6) | 0.014905 | 1.008 | 0.581 | 0.4269 |
| S2 | 0.016758 | 17.06 | 11.96 | 5.095 |
| Total G1 | 0.034016 | 31.8 | 34.87 | −3.068 |
| FA2G2S2 | 0.034085 | 2.454 | 1.926 | 0.528 |
| Fucosylated | 0.035203 | 80.69 | 86.75 | −6.06 |
| FA2BG1 (1, 3) | 0.051792 | 0.6962 | 0.5552 | 0.141 |
| FA2G1S1 (1, 3) | 0.090103 | 2.909 | 2.453 | 0.4558 |
| A2G2S2 (1, 3) | 0.09808 | 10.52 | 6.876 | 3.642 |
| FA2G1 (1, 3) | 0.257673 | 7.754 | 8.796 | −1.042 |
| FA1 | 0.264877 | 0.09922 | 0.05675 | 0.04247 |
| Total G2 | 0.299069 | 44.73 | 39.66 | 5.065 |

Lower levels of the following IgG N-glycans distinguish acute Lyme disease from treated Lyme disease: FA2G1 (1,6), G1, Total G, fucosylated, FA2G1 (1,3). Higher levels of the following IgG N-glycans distinguish acute Lyme disease from treated Lyme disease: FA2G1S-(1,6), A2G2S1 (1,6), A2G2S2 (1,6), S2, FA2G2S2, FA2BG1 (1,3), FA2G1S1 (1,3), A2G2S2 (1,3), FA1, Total G2.

TABLE 3
IgG N-glycan profiles, Acute Lyme
Disease Vs. Rheumatoid Arthritis

| | Adjusted | Mean of | Mean of | |
|---|---|---|---|---|
| | P Value | Acute LD | RA | Difference |
| FA1 | <0.000001 | 0.09922 | 0.2867 | −0.1875 |
| FA2G1S1 (1, 6) | <0.000001 | 0.3141 | 0.5969 | −0.2827 |
| A2G2S1 (1, 6) | 0.000017 | 0.6416 | 1.046 | −0.404 |
| FA2G1S1 (1, 3) | 0.000083 | 2.909 | 3.749 | −0.8398 |
| FA2BG1 (1, 3) | 0.000107 | 0.6962 | 0.9483 | −0.2521 |
| FA2G2 | 0.013794 | 11.34 | 8.467 | 2.876 |
| FA2BG2S2 | 0.035133 | 2.268 | 2.728 | −0.4607 |
| G1 | 0.054957 | 29.68 | 26.78 | 2.901 |
| G2 | 0.079123 | 14.68 | 12.07 | 2.612 |
| FA2G1 (1, 3) | 0.125296 | 7.754 | 6.66 | 1.094 |
| FA2G1 (1, 6) | 0.13853 | 15.15 | 13.57 | 1.582 |
| G0 | 0.155632 | 22.3 | 27.55 | −5.249 |
| FA2 | 0.158483 | 17.06 | 21.38 | −4.319 |
| FA2G2S1 | 0.20103 | 7.774 | 6.438 | 1.336 |
| Bisecting | 0.284339 | 16.64 | 18.36 | −1.725 |

Lower levels of the following IgG N-glycans distinguish acute Lyme disease from rheumatoid arthritis: FA1, FA2G1S1 (1,6), A2G2S1 (1,6), FA2G1S1 (1,3), FA2BG1 (1,3), FA2BG2S2, G0, FA2, Bisecting. Higher levels of the following IgG N-glycans distinguish acute Lyme disease from rheumatoid arthritis: FA2G2, G1, G2, FA2G1 (1,3), FA2G1 (1,6), FA2G2S1.

TABLE 4
IgG N-glycan profiles, Acute Lyme Disease Vs. Syphilis

| | Adjusted | Mean of | Mean of | |
|---|---|---|---|---|
| | P Value | Acute LD | Syphilis | Difference |
| FA2BG2S2 | 0.007712 | 2.268 | 2.832 | −0.5646 |
| FA2G2S2 | 0.00972 | 2.454 | 3.074 | −0.6205 |

Lower levels of the following IgG N-glycans distinguish acute Lyme disease from syphilis: FA2BG2S2, FA2G2S2.

TABLE 5
IgG N-glycan profiles, Acute Lyme Disease Vs. Lupus

| | Adjusted | Mean of | Mean of | |
|---|---|---|---|---|
| | P Value | Acute LD | Lupus (SLE) | Difference |
| FA2BG2S2 | 0.001371 | 2.268 | 3.189 | −0.9214 |
| Bisecting | 0.017108 | 16.64 | 19.46 | −2.823 |
| G2 | 0.032814 | 14.68 | 11.44 | 3.239 |
| FA2B | 0.132355 | 4.272 | 5.507 | −1.235 |
| FA2G2 | 0.193314 | 11.34 | 9.035 | 2.309 |
| S1 | 0.270371 | 16.21 | 14.32 | 1.897 |
| G0 | 0.290781 | 22.3 | 27.37 | −5.068 |
| FA1 | 0.290781 | 0.09922 | 0.05409 | 0.04513 |

Lower levels of the following IgG N-glycans distinguish acute Lyme disease from lupus: FA2BG2S2, Bisecting, FA2B, G0. Higher levels of the following IgG N-glycans distinguish acute Lyme disease from lupus: G2, FA2G2, S1, FA1.

TABLE 6
IgG N-glycan profiles, Acute Lyme Disease Vs. Fibromyalgia

| | Adjusted | Mean of | Mean of | |
|---|---|---|---|---|
| | P Value | Acute LD | Fibromyalgia | Difference |
| FA1 | 0.028568 | 0.09922 | 0.03761 | 0.06161 |
| FA2BG2S2 | 0.149748 | 2.268 | 2.71 | −0.4428 |

Lower levels of the following IgG N-glycans distinguish acute Lyme disease from fibromyalgia: FA2BG2S2. Higher levels of the following IgG N-glycans distinguish acute Lyme disease from fibromyalgia: FA1.

TABLE 7
IgM N-glycan profiles, Control Vs. Acute Lyme Disease

| | Adjusted | Mean of | Mean of | |
|---|---|---|---|---|
| | P Value | Control | Acute LD | Difference |
| S2 | 0.000061 | 17.99 | 22.7 | −4.712 |
| FM4A1 | 0.000066 | 0.6525 | 0.4858 | 0.1666 |
| Man 7 | 0.000333 | 2.808 | 2.289 | 0.5183 |
| Mannose | 0.000708 | 24.97 | 21.9 | 3.068 |
| Bisecting | 0.000843 | 40.37 | 34.86 | 5.513 |
| FA2BG2 | 0.001025 | 2.377 | 1.938 | 0.4393 |
| FA2BG2S1 | 0.001116 | 25.67 | 21.46 | 4.21 |
| M5 | 0.004546 | 7.081 | 6.109 | 0.9713 |
| Fucosylation | 0.005958 | 46.04 | 42.52 | 3.521 |
| Man 6 D3 | 0.00893 | 8.473 | 7.055 | 1.418 |
| A2G2S2 (3, 3) | 0.017596 | 5.292 | 7.972 | −2.679 |
| FA2G2S2 | 0.03447 | 4.391 | 6.202 | −1.811 |
| Total G3 | 0.060644 | 1.749 | 3.183 | −1.435 |
| M8 | 0.084759 | 0.4344 | 0.7015 | −0.2671 |
| S3 | 0.100376 | 1.545 | 2.686 | −1.142 |
| Total G2 | 0.132866 | 65.6 | 67.57 | −1.971 |

Increases in the following IgM N-glycans over normal indicate acute Lyme disease: S2, A2G2S2 (3,3), FA2G2S2, Total G3, M8, S3, Total G2. Decreases in the following IgM N-glycans over normal indicate acute Lyme disease: FM4A1, Man 7, Mannose, Bisecting, FA2BG2, FA2BG2S1, M5, Fucosylation, Man 6 D3.

TABLE 8
IgM N-glycan profiles, Acute Lyme
Disease Vs. Lyme Disease, Treated

| | Adjusted | Mean of | Mean of | |
|---|---|---|---|---|
| | P Value | Acute LD | Treated LD | Difference |
| S2 | 0.011216 | 22.7 | 18.57 | 4.131 |
| Mannose | 0.013277 | 21.9 | 25 | −3.094 |
| M5 | 0.019801 | 6.109 | 7.192 | −1.083 |
| Man 7 | 0.032459 | 2.289 | 2.739 | −0.4493 |
| Man 6 D3 | 0.049381 | 7.055 | 8.626 | −1.571 |

Lower levels of the following IgM N-glycans distinguish acute Lyme disease from treated Lyme disease: Mannose, M5, Man 7, Man 6 D3. Higher levels of the following IgM N-glycans distinguish acute Lyme disease from treated Lyme disease: S2.

TABLE 9
IgM N-glycan profiles, Acute Lyme
Disease Vs. Rheumatoid Arthritis

| | Adjusted | Mean of | Mean of | |
|---|---|---|---|---|
| | P Value | Acute LD | RA | Difference |
| FA2BG2S2 | <0.000001 | 5.266 | 7.467 | −2.201 |
| A3G3S2 | 0.000002 | 0.497 | 1.524 | −1.027 |
| FA2G2S1 | 0.000235 | 16.09 | 12.04 | 4.053 |
| FA2BG1S1 (1, 3) | 0.000446 | 1.549 | 2.232 | −0.683 |
| Man 6 D1 or 2 | 0.007487 | 2.91 | 2.167 | 0.7432 |
| Mannose | 0.007871 | 21.9 | 18.89 | 3.011 |
| Total G3 | 0.00864 | 3.183 | 5.761 | −2.578 |
| M8 | 0.028261 | 0.7015 | 1.044 | −0.3422 |
| Fucosylation | 0.036229 | 42.52 | 46.5 | −3.985 |
| S2 | 0.046549 | 22.7 | 26.58 | −3.881 |
| Man 6 D3 | 0.064435 | 7.055 | 5.749 | 1.306 |
| M5 | 0.087683 | 6.109 | 5.302 | 0.8074 |
| S1 | 0.093565 | 46.91 | 43.31 | 3.598 |
| A3G3S3 (3, 6, 6) | 0.110644 | 1.452 | 2.35 | −0.898 |
| M4G1S1 + A3G2 | 0.117185 | 2.648 | 2.248 | 0.4009 |
| A2G2S1 | 0.137645 | 3.238 | 2.702 | 0.5356 |

Lower levels of the following IgM N-glycans distinguish acute Lyme disease from rheumatoid arthritis: FA2BG2S2, A3G3S2, FA2BG1S1 (1,3), Total G3, M8, Fucosylation, S2, A3G3S3 (3,6,6). Higher levels of the following IgM N-glycans distinguish acute Lyme disease from rheumatoid arthritis: FA2G2S1, Man 6 D1 or 2, Mannose, Man 6 D3, M5, S1, M4G1S1+A3G2, A2G2S1.

TABLE 10
IgM N-glycan profiles, Acute Lyme Disease Vs. Syphilis

| | Adjusted | Mean of | Mean of | |
|---|---|---|---|---|
| | P Value | Acute LD | Syphilis | Difference |
| A2BG2S2 | 0.011635 | 1.296 | 1.043 | 0.2522 |
| FA2BG2S2 | 0.131762 | 5.266 | 6.227 | −0.9617 |

Lower levels of the following IgM N-glycans distinguish acute Lyme disease from syphilis: FA2BG2S2. Higher levels of the following IgM N-glycans distinguish acute Lyme disease from syphilis: A2BG2S2.

TABLE 11
IgM N-glycan profiles, Acute Lyme Disease Vs. Lupus

| | Adjusted | Mean of | Mean of | |
|---|---|---|---|---|
| | P Value | Acute LD | Lupus (SLE) | Difference |
| FA2BG2S2 | 0.00008 | 5.266 | 6.788 | −1.522 |
| FA2G2S1 | 0.000314 | 16.09 | 11.58 | 4.513 |
| Man 6 D1 or 2 | 0.00316 | 2.91 | 2.099 | 0.811 |
| FA2BG1S1 (1, 3) | 0.016562 | 1.549 | 2.244 | −0.6951 |
| FA2BG1 | 0.052264 | 1.424 | 1.847 | −0.4228 |

Lower levels of the following IgM N-glycans distinguish acute Lyme disease from lupus: FA2BG2S2, FA2BG1S1 (1,3), FA2BG1. Higher levels of the following IgM N-glycans distinguish acute Lyme disease from lupus: FA2G2S1, Man 6 D1 or 2.

TABLE 12
IgM N-glycan profiles, Acute Lyme Disease Vs. Fibromyalgia

| | Adjusted | Mean of | Mean of | |
|---|---|---|---|---|
| | P Value | Acute LD | Fibromyalgia | Difference |
| FA2BG2S2 | 0.002387 | 5.266 | 6.459 | −1.194 |
| Fucosylation | 0.018187 | 42.52 | 46.74 | −4.22 |
| Bisecting | 0.086224 | 34.86 | 39.67 | −4.804 |
| M4G1S1 + A3G2 | 0.129056 | 2.648 | 2.192 | 0.4567 |

Lower levels of the following IgM N-glycans distinguish acute Lyme disease from fibromyalgia: FA2BG2S2, Fucosylation, Bisecting. Higher levels of the following IgM N-glycans distinguish acute Lyme disease from fibromyalgia: M4G1 S1+A3G2.

TABLE 13
Total Serum N-glycan profiles, Control Vs. Acute Lyme Disease

| | Adjusted | Mean of | Mean of | |
|---|---|---|---|---|
| | P Value | Control | Acute LD | Difference |
| FA2BG1 (1, 6) | 0.000541 | 0.7927 | 0.4786 | 0.3141 |
| G1 | 0.000641 | 5.084 | 3.263 | 1.822 |
| Total G1 | 0.000774 | 6.708 | 4.664 | 2.044 |
| FA2G1 (1, 3) | 0.001609 | 1.386 | 0.8595 | 0.5268 |
| FA2BG2 | 0.002703 | 0.8362 | 0.6189 | 0.2173 |
| FA2G1 (1, 6) | 0.009458 | 2.464 | 1.637 | 0.8271 |
| Fucosylated | 0.015862 | 23.17 | 19.76 | 3.406 |
| GO | 0.028997 | 4.332 | 2.98 | 1.352 |
| G2 | 0.042739 | 2.806 | 2.046 | 0.7594 |
| S2 | 0.042739 | 50.69 | 54.26 | −3.568 |
| A2G2S2 (1, 3) | 0.067243 | 37.85 | 41.51 | −3.661 |
| Man 5 + FA2B | 0.070774 | 1.331 | 0.9832 | 0.3475 |
| FA2 | 0.070774 | 2.864 | 1.892 | 0.9723 |

Increases in the following total serum N-glycans over normal indicate acute Lyme disease: S2, A2G2S2 (1,3). Decreases in the following total serum N-glycans over normal indicate acute Lyme disease: FA21BG1 (1,6), G1, Total G1, FA2G1 (1,3), FA2BG2, FA2G1 (1,6), Fucosylated, G0, G2, Man 5+FA2B, FA2.

TABLE 14
Total Serum N-glycan profiles, Acute Lyme
Disease Vs. Lyme Disease, Treated

| | Adjusted | Mean of | Mean of | |
|---|---|---|---|---|
| | P Value | Acute LD | Treated LD | Difference |
| A3G3S2 (3, 6, 6) | 0.001565 | 0.5414 | 0.6752 | −0.1338 |
| A3G3S2 (3, 3, 6) | 0.008304 | 0.7099 | 0.9538 | −0.2439 |
| A2G2S2 (1, 3) | 0.013203 | 41.51 | 36.88 | 4.628 |
| A3G3S3 (6, 6, 6) | 0.036718 | 0.9928 | 1.288 | −0.2949 |
| A4G4S3 (3, 6, 6, 6) | 0.060682 | 0.1993 | 0.2751 | −0.07587 |
| FA2BG1 (1, 6) | 0.118966 | 0.4786 | 0.6666 | −0.188 |
| G2 | 0.132246 | 2.046 | 2.783 | −0.7368 |
| S2 | 0.148 | 54.26 | 50.8 | 3.459 |

Lower levels of the following total serum N-glycans distinguish acute Lyme disease from treated Lyme disease: A3G3S2 (3,6,6), A3G3S2 (3,3,6), A3G3S3 (6,6,6), A4G4S3 (3,6,6,6), FA21G1 (1,6), G2. Higher levels of the following total serum N-glycans distinguish acute Lyme disease from treated Lyme disease: A2G2S2 (1,3), S2.

TABLE 15
Total Serum N-glycan profiles, Acute
Lyme Disease Vs. Rheumatoid Arthritis

| | Adjusted | Mean of | Mean of | |
|---|---|---|---|---|
| | P Value | Acute LD | RA | Difference |
| FA2G1S1 | 0.001326 | 1.157 | 1.509 | −0.3517 |
| A2G2S1 (1, 6) | 0.00284 | 0.4429 | 0.6364 | −0.1934 |
| A3G3S3 (3, 6, 6) | 0.00299 | 0.5405 | 0.3298 | 0.2107 |
| A4G4S3 (3, 6, 6, 6) | 0.013325 | 0.1993 | 0.125 | 0.07429 |
| A4G4S3 (6, 6, 6, 6) | 0.017327 | 0.5552 | 0.4083 | 0.1469 |
| A3G3S2 (3, 3, 3) | 0.042049 | 0.76 | 0.4973 | 0.2627 |
| A4G4S4 (3, 6, 6, 6) | 0.043252 | 1.597 | 1.253 | 0.3447 |
| Total G4 | 0.087348 | 5.805 | 4.751 | 1.053 |

Lower levels of the following total serum N-glycans distinguish acute Lyme disease from rheumatoid arthritis: FA2G1S1, A2G2S1 (1,6). Higher levels of the following total serum N-glycans distinguish acute Lyme disease from rheumatoid arthritis: A3G3S3 (3,6,6), A4G4S3 (3,6,6,6), A4G4S3 (6,6,6,6), A3G3S2 (3,3,3), A4G4S4 (3,6,6,6), Total G4.

TABLE 16
Total Serum N-glycan profiles, Acute Lyme Disease Vs. Syphilis

| | Adjusted | Mean of | Mean of | |
|---|---|---|---|---|
| | P Value | Acute LD | Syphilis | Difference |
| A3G3S2 (3, 6, 6) | 0.000022 | 0.5414 | 0.3704 | 0.1709 |
| A3G3S2 (3, 3, 3) | 0.000535 | 0.76 | 0.3986 | 0.3614 |
| Al (Class) | 0.000889 | 0.1734 | 0.08862 | 0.08482 |
| A3G3S3 (3, 6, 6) | 0.000998 | 0.5405 | 0.3238 | 0.2167 |
| A1 | 0.001346 | 0.1373 | 0.06413 | 0.07318 |
| A4G4S3 (6, 6, 6, 6) | 0.001538 | 0.5552 | 0.3971 | 0.1581 |
| FA2G1S1 | 0.002635 | 1.157 | 1.498 | −0.3412 |
| FA2G1 (1, 6) | 0.00878 | 1.637 | 2.762 | −1.125 |
| Total G1 | 0.009595 | 4.664 | 6.849 | −2.185 |
| A4G4S3 (3, 6, 6, 6) | 0.013066 | 0.1993 | 0.123 | 0.07629 |
| Total G3 | 0.023321 | 19.45 | 17.02 | 2.433 |
| G1 | 0.023321 | 3.263 | 5.123 | −1.86 |
| FA2 | 0.026381 | 1.892 | 3.224 | −1.332 |
| Fucosylated | 0.026381 | 19.76 | 23.95 | −4.188 |
| | 0.030513 | 2.98 | 4.742 | −1.762 |
| FM4A1G1S1 | 0.055983 | 3.449 | 4.181 | −0.7323 |
| FA2G1 (1, 3) | 0.057344 | 0.8595 | 1.327 | −0.4673 |
| A4G4S4 (3, 3, 3, 3) | 0.058673 | 0.2495 | 0.1611 | 0.08834 |
| A2 | 0.062332 | 70.25 | 72.86 | −2.612 |
| FA2BG2 | 0.082205 | 0.6189 | 0.8116 | −0.1927 |
| G2 | 0.08956 | 2.046 | 2.888 | −0.8421 |
| S3 | 0.113046 | 18.43 | 16.35 | 2.082 |
| Total G4 | 0.124796 | 5.805 | 4.861 | 0.9432 |
| Mannosylated | 0.12896 | 5.544 | 6.785 | −1.241 |
| S1 | 0.133229 | 13.02 | 14.63 | −1.611 |

Lower levels of the following total serum N-glycans distinguish acute Lyme disease from syphilis: FA2G1S1, FA2G1 (1,6), Total G1, G1, FA2, Fucosylated, FM4A1G1S1, FA2G1 (1,3), A2, FA2BG2, G2, Mannosylated, S1. Higher levels of the following total serum N-glycans distinguish acute Lyme disease from syphilis: A3G32 (3,6,6), A3G3S2 (3,3,3), A1 (Class), A3G3S3 (3,6,6), A1, A4G4S3 (6,6,6,6), A4G4S3 (3,6,6,6), Total G3, A4G4S4 (3,3,3,3), S3, Total G4.

TABLE 17
Total Serum N-glycan profiles, Acute Lyme Disease Vs. Lupus

| | Adjusted | Mean of | Mean of | |
|---|---|---|---|---|
| | P Value | Acute LD | Lupus (SLE) | Difference |
| A3G3S2 (3, 6, 6) | <0.000001 | 0.5414 | 0.3512 | 0.1901 |
| A3G3S3 (3, 6, 6) | 0.000018 | 0.5405 | 0.2628 | 0.2777 |
| A2G2S1 (1, 6) | 0.000824 | 0.4429 | 0.6228 | −0.1799 |
| A3G3S2 (3, 3, 3) | 0.00732 | 0.76 | 0.428 | 0.332 |
| Total G3 | 0.032313 | 19.45 | 16.8 | 2.653 |
| A2 | 0.043751 | 70.25 | 73.13 | −2.877 |
| FA2BG1 (1, 6) | 0.056271 | 0.4786 | 0.7766 | −0.298 |
| FA2G1S1 | 0.082893 | 1.157 | 1.412 | −0.2546 |
| A3G3S2 (3, 3, 6) | 0.090176 | 0.7099 | 0.5009 | 0.209 |

Lower levels of the following total serum N-glycans distinguish acute Lyme disease from lupus: A2G2S1 (1,6), A2, FA2BG1 (1,6), FA2G1 S1. Higher levels of the following total serum N-glycans distinguish acute Lyme disease from lupus: A3G3S2 (3,6,6), A3G3S3 (3, 6,6), A3G3S2 (3,3,3), Total G3, A3G3S2 (3,3,6).

TABLE 18
Total Serum N-glycan profiles, Acute
Lyme Disease Vs. Fibromyalgia

| | Adjusted | Mean of | Mean of | |
|---|---|---|---|---|
| | P Value | Acute LD | Fibromyalgia | Difference |
| A3G3S2 (3, 6, 6) | 0.023702 | 0.5414 | 0.42 | 0.1214 |
| A3G3S3 (3, 6, 6) | 0.041444 | 0.5405 | 0.3483 | 0.1922 |

Higher levels of the following total serum N-glycans distinguish acute Lyme disease from fibromyalgia: A3G3S2 (3,6,6), A3G3S3 (3,6,6).

### Example 4: Host Glycosylation of Immunoglobulins Impairs the Immune Response to Acute Lyme Disease

Lyme disease is caused by the bacteria Borreliella burgdorferi sensu lato (Bb) transmitted to humans from the bite of an infected Ixodes tick. Current diagnostics for Lyme disease are insensitive at the early disease stage and they cannot differentiate between active infections and people with a recent history of antibiotic-treated Lyme disease.

In the present study, machine learning technology was utilized to improve the prediction of acute Lyme disease and identify sialic acid and galactose sugar structures (N-glycans) on immunoglobulins associated specifically at time points during acute Lyme disease time. A plate-based approach was developed to analyze sialylated N-glycans associated with anti-Bb immunoglobulins. This multiplexed approach quantitates the abundance of Bb-specific IgG and the associated sialic acid, yielding an accuracy of 90% in a powered study.

It was demonstrated that immunoglobulin sialic acid levels increase during acute Lyme disease and following antibiotic therapy and a 3-month convalescence, the sialic acid level returned to that found in healthy control subjects (p<0.001). Furthermore, the abundance of sialic acid on Bb-specific IgG during acute Lyme disease impaired the host's ability to combat Lyme disease via lymphocytic receptor FcγRIIIa signaling and C1q-mediated complement deposition. After enzymatically removing the sialic acid present on Bb-specific antibodies, the induction of cytotoxicity from acute Lyme disease patient antigen-specific IgG was significantly improved.

Taken together, Bb-specific immunoglobulins contain increased sialylation which impairs the host immune response during acute Lyme disease. Furthermore, this Bb specific immunoglobulin sialyation found in acute Lyme disease begins to resolve following antibiotic therapy and convalescence.

### Example 4-1

Lyme disease is caused by the spirochete Borreliella (previously named Borrelia) burgdoiferi sensu lato (Bb) transmitted to humans from the bite of an infected Ixodes tick. The bacterium disseminates to multiple organ systems rapidly and when the erythema migrans rash is absent or unrecognized, acute Lyme disease is challenging to diagnose. The disease is endemic in the upper Midwest and Northeastern US, and incidence rates continue to rise. For decades, clinical laboratories have assessed the humoral response to Lyme disease using western immunoblots and ELISA methods. Serological assays determine the abundance of antigen-specific antibodies; thus, they cannot differentiate acute Lyme disease (ALD) cases from late-stage Lyme disease or people with a recent history of resolved Lyme disease. It was demonstrated that the N-glycosylation of IgG from ALD patients differed from the canonical pro-inflammatory profile observed in other acute bacterial infections. N-glycosylation of proteins is the most common post-translational modification observed in circulating proteins, and the structure, stereochemistry, and charge of the N-glycan motifs confer different functionality and receptor affinity. With this fact in mind, the present study sought to determine if an assessment of host antibody N-glycosylation can improve the diagnostic accuracy of acute Lyme disease and provide more information about the immunologic response to ALD. To do so, the present study analyzed total IgG and IgM N-glycomes from cohorts of ALD, convalescent treated Lyme disease, Lyme-endemic healthy controls, non-Lyme-endemic healthy controls, and patients with rheumatoid arthritis, syphilis, fibromyalgia, and lupus.

IgG N-glycan profiles consist of biantennary complex N-glycans that range in size and charge depending on the presence of N-acetylglucosamine, galactose, core-fucose, and sialic acid. During health, the posttranslational addition of N-glycans to the single conserved glycosylation site on the heavy chain of human IgG yields a reproducible profile. IgG N-glycans determine the affinity for cellular Fc receptors and can promote C1q mediated complement activation. Thus, IgG N-glycans are an excellent resource to understand acute disease responses. IgM glycosylation profiles are markedly different from IgG and less well studied. The heavy chain of IgM has five separate N-glycosylation sites containing complex-type, hybrid N-glycans, and highly-mannosylated Nglycans. In addition, IgM monomers combine to form a pentamer in serum, playing a vital role in combating bacteria and viruses during early immune responses. For example, the classical complement pathway is influenced by IgM N-glycosylation. N-glycans decorating IgG and IgM are sequentially added by B lymphocyte glycosyltransferases (GTs) in a highly regulated fashion. Expression of GTs in B lymphocytes is altered by a growing list of cytokines including INF-g, TNF-α, IL-17A, TH17, and IL-21 as well as environmental factors such as trans-retinoic acid and CpG oligodeoxynucleotide. Thus, extracellular stimuli can alter B cell GT expression, resulting in different immunoglobulin N-glycan profiles. Because Bb is known to disrupt the germinal centers of lymph nodes during acute infection, we hypothesized that the N-glycosylation of serum IgG and IgM could reflect the resulting dysregulation in the immune response.

This is the first comprehensive report on acute Lyme disease host immunoglobulin glycosylation. The initial analysis of total IgG and total IgM N-glycomes revealed ALD patients contained significantly more sialylated species compared to the treated Lyme disease cohort. To study this phenomenon in detail, the present study developed an antigen-specific lectin multiplex assay to track the sialylation of anti-Bb immunoglobulins. Finally, the present study demonstrates the inhibitory role of IgG N-glycan sialylation during the host immune response to Bb infection using two in vitro assays.

### Example 4-2: Methods

### Patient Samples

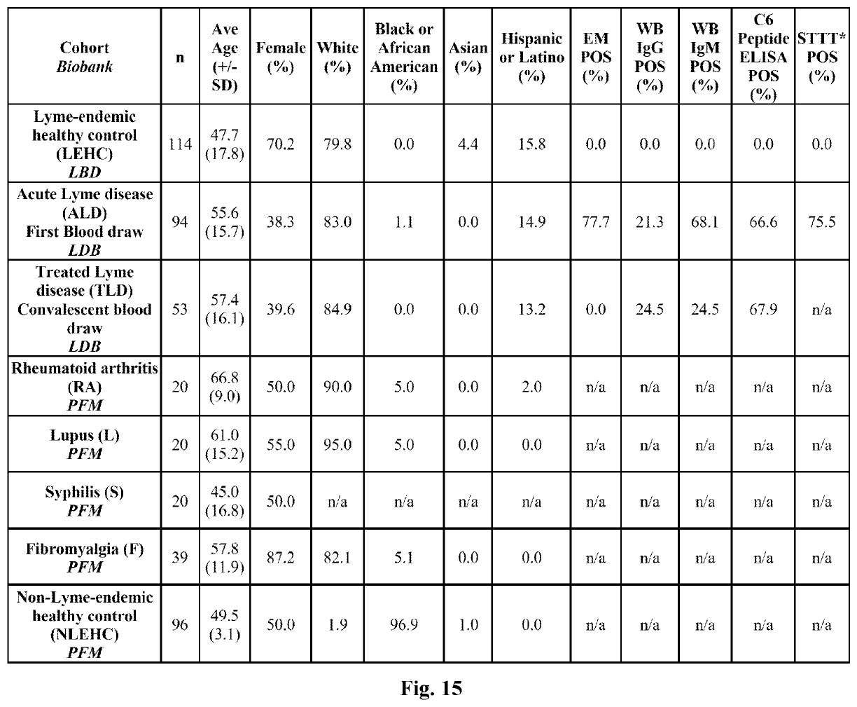

Sera from Lyme-endemic healthy controls (LEHC) and acute Lyme disease (ALD) were collected from consenting individuals. In addition, an optional second draw of sera was collected from a subset of the acute Lyme disease patients following a 2-3-month convalescence period. All patients who were diagnosed with ALD in this study were promptly treated with standard antibiotic therapy. The convalescent timepoint sera is referred to as the ‘treated Lyme disease’ (TLD) cohort within this manuscript. The present study selected the TLD nomenclature because this cohort represents patients who are no longer considered infected with the Bb bacterium. All LDB samples were assessed by standard two-tiered testing (STTT), C6 peptide ELISA (Oxford Immunotec, Marlborough, MA), and by PCR for Borrelia and other tick-borne infections. Samples were provided in a blinded and de-identified manner and analyzed under Drexel's IRB #1808006553. ALD and LEHC patients in this study tested PCR-negative for other tick-borne infections including Anaplasma phagocytophilum, Babesia microti, and Ehrlichia chaffeensis, which were the main limit to the ALD sample size. In addition, human sera samples collected from consenting adults over the age of 18 classified as non-Lyme endemic healthy controls (NLEHC) or diagnosed with rheumatoid arthritis (RA), secondary syphilis (S), fibromyalgia (F), or systemic erythematous lupus (L) were purchased from the Precision for Medicine biobank in a deidentified format. Cohort characteristics from both biobanks including distribution of age, sex, race, and ethnicity, are listed in FIG. 15.

### Isolation of Total IgG

Total IgG was isolated from 20 μL of plasma using a Protein G spin plate as described by the manufacturer (ThermoFisher, MA). Four 200 μL 1×PBS washes removed unbound plasma protein using a vacuum manifold apparatus. Next, IgG was eluted by incubating 150 μL of 0.1M glycine HCl pH 2-3 for 5 minutes at room temperature. The eluate was collected into a 96-well 2 mL collection plate pre-loaded with 15 μL of 1.5M Tris pH 8 to neutralize the glycine elution buffer. The wash process was repeated a second time to ensure a high yield of IgG. The resulting 315 μL of the neutralized eluate was concentrated and buffer-exchanged to 20 μL of 1×PBS using Amicron Ultra-0.5 centrifugal filter—10 kDa MWCO (Millipore) following the manufacturer's instructions. NanoDrop 1000 spectrophotometer readings monitored protein yield through the protein isolation process.

### Isolation of Total IgM

Total IgM was isolated from plasma by incubating 80 μL of goat anti-IgM agarose-conjugated agarose beads (A9935, Millipore Sigma, MA) with 80 μL plasma and 100 μL 1×PBS for 2 hours at room temperature. Following the incubation, the solution was transferred to a 1.2 μm MultiScreen HTS 96-well filter plate. Four 200 μL 1×PBS washes removed unbound plasma protein using a vacuum manifold apparatus. Next, IgM was eluted by incubating 150p of 0.1M glycine HCl pH 2-3 for 5 minutes at room temperature. The eluate was collected into a 96-well 2 mL collection plate pre-loaded with 15 μL of 1.5M Tris pH 8 to neutralize the glycine elution buffer. The wash process was repeated a second time to ensure a high yield of IgM. The resulting 315 μL of the neutralized eluate was concentrated and buffer-exchanged to 20 μL of 1×PBS using Amicron Ultra-0.5 centrifugal filters—10 kDa MWCO (Millipore) as described by the manufacturer. NanoDrop 1000 spectrophotometer readings monitored protein yield through the protein isolation process

### Immunoglobulin N-Glycan Analysis

N-glycans from IgG and IgM were released, labeled, and analyzed as described previously using the Waters GlycoWorks RapiFluor MS procedure, adapted for PCR tube volumes. Briefly, samples were denatured using the RapiGest reagent for 5 minutes at 95° C. using a PCR thermocycler. Next, glycoprotein samples were deglycosylated using PNGase F for 6 minutes at 60° C. using a PCR thermocycler. Afterward, samples were labeled with RapiFluor label (RFMS) for 5 minutes at room temperature. A solid-phase extraction (SPE) clean-up module isolated RFMS labeled N-glycans which were then eluted into a 96-well 2 mL Waters ANSI plate capped with a PFTE 96-well membrane top for high-throughput N-glycan analysis. An ACQUITY Premier UPLC System was used following the setting and protocol. Briefly, an ACQUITY UPLC BEH Amide Column, 130A, 1.7 m, 2.1 mm×50 mm column (Waters, MA) was used to chromatographically separate N-glycans during the 18.3 min run employing a gradient of 50 mM Ammonium Formate pH 4.4 (Waters) made with LC-MS Water (Millipore), LC-MS ACN (VWR, Honeywell) 25%-75% gradient transitioning over 12 min to 60%-40%. N-glycans separated by charge and stereochemistry were quantitated using Waters AQUITY Fluorescent detector set to 265/425 em/ex, 10 Hz using Empower 3 software. Lastly, N-glycan identity was confirmed using a Waters AQUITY QDa Mass spectrometer. The resulting UPLC fluorescent trace was analyzed with Empower v3.3.1 software, UPLC trace percentarea was combined with collected MS-spectra to identify eluted peaks.

### Antigen-Specific Lectin Multiplex Assay

High-binding 96-well plates (Nest Biotechnology, 514201) were incubated with the recombinantly expressed Borrelia burgdorferi (Bb) antigens: Outer Surface Protein C (OspC) protein (Rockland, 000-001-C11) and Variable Lipoprotein Surface-Exposed (VlsE) protein (Rockland, 000-001-C33). The OspC and VlsE antigens were diluted in 1×PBS buffer such that each of the odd-numbered columns of the 96-well plate received 50 ng of each antigen in a final volume of 100 μL. Even-numbered columns of the 96-well plate were incubated with 100 μL of 1×PBS lacking antigen to control for background signals from each sera sample. The 100 μL of antigens and 1×PBS were incubated on the plate at 4° C. for 12 hours. Plates were then aspirated, washed once with 300 μL of 1×PBS 1 mM CaCl₂) 0.1% Tween-20, and blocked with 300 μL 1×CarboFree Block (Vector, SP-5040) for 2 hours at room temperature with orbital shaking. Next, the 1×CarboFree block was aspirated, and 100 μL of 1:20 diluted sera (5 μL sera in 95 μL 1×PBS) was added to both the antigen-coated odd-numbered columns and the even-numbered 1×PBS background control wells. Two wells from each plate were reserved for a 1:20 dilution of a pooled stock of acute Lyme disease (ALD) sera which were used to normalize each plate's final anti-OspC/VlsE IgG*SNA signal herein referred to as the GlycoLyme score. The 1:20 diluted sera was incubated at 4° C. for 12 hours. Next, the plate was aspirated and washed five times with 300 μL of 1×PBS 1 mM CaCl₂) 0.1% Tween-20, followed by the addition of 100 μL of 1 ng/μL biotinylated Sambucus Nigra (SNA) Lectin (Vector Laboratories, B-1305-2) diluted in 1×PBS with 1 mM CaCl₂) and 0.1% Tween-20 incubated at room temperature for 1 hour. Next, the plate contents were aspirated, washed three times with 300 μL of 1×PBS (with 1 mM CaCl₂) and 0.1% Tween-20), and incubated with 100 μL of 1 ng/μL IRDye 800CW Streptavidin (LiCor, 926-32230) diluted in 1×PBS (with 1 mM CaCl₂) 0.1% and Tween-20) incubated at room temperature for 1 hour. Next, the plate contents were aspirated, washed three times with 300 μL of 1×PBS (with 1 mM CaCl₂) and 0.1% Tween-20), and incubated with 100 μL of 0.05 ng/μL IRDye 680LT goat anti-human IgG (LiCor, 926-68078) diluted in 1×PBS at room temperature for 1 hour. Next, the plate contents were aspirated, washed three times with 300 μL of 1×PBS (with 1 mM CaCl₂) and 0.1% Tween-20) and imaged using a LiCor Odyssey CLx on the 700 and 800 IR laser channels with 84 m scanning resolution set to medium quality with a 3.5 mm offset. The resulting data were quantitated using LiCor Image Studio.

### Exoglycosidase Digestion

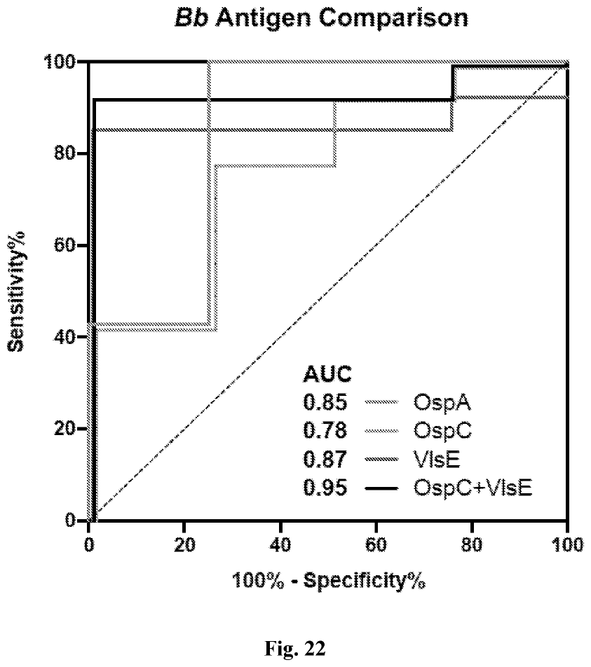

Sera samples were pooled to obtain enough volume required for the validation of the exoglycosidase digestions, cellular cytotoxicity, and complement deposition assays. Pooled samples were then diluted 1:1 in 1×PBS, divided into three equal aliquots, and incubated in a heat block at 37° C. for 12 hours with one of the following per aliquot: 10 μL of 1×PBS as a mock-digestion control (Mock), 10 μL of 1 g/μL PNGase F PRIME-LY (PNG) (Nzyme Scientifics), or 4 μL of 50 U/μL a 2-3,6,8 sialidase (SA) (New England BioLabs, MA, P0720L) as described by the manufacturer. Following the digestion or mock-digestion with 1×PBS, the pooled sera were aliquoted into 30 μL stocks and stored at −80° C. until use. To validate the exoglycosidase digestions, anti-VlsE immunoglobulins were isolated using the plate-based lectin approach detailed above and interrogated for 1 ng/μL SNA reactivity shown in FIG. 22.

### Antigen-Specific Cellular Cytotoxicity Assay (ADCC)

This ADCC assay was adapted from the approach detailed by Chen et al. (J Immunol Methods, 2014. 414: p. 69-81) and Yu et al. (Journal of immunological methods, 2019. 473: p. 112630-112630) using a ADCC Reporter BioAssay procedure (Promega, 7015). In brief, a 96-well high-binding ELISA plate was coated with 50 ng/well of the VlsE antigen and blocked as described previously. Pooled sera samples were diluted to 1:20 in 1×PBS, and 100 μL of each diluted sample was loaded on the plate in triplicate. Next, samples were incubated at 4° C. for 12 hours, washed three times with 300 μL of 1×PBS (with 1 mM CaCl₂) and 0.1% Tween-20), and washed three times with 300 μL of 1×PBS to remove Tween-20. Next, ADCC effector cells engineered from Jurkat cell lines stably expressing the FcγRIIIa receptor, V158 (high affinity) variant, and an NFAT response element driving expression of firefly luciferase were added to the plate as described by the manufacturer and incubated at 37° C. with 5% C02 and humidity for 6 hours. Next, the contents of the plate were transferred to a white, flat-bottomed DeepWell LumaPlate-96 (Revvity, 6005630) containing 75 μL of the reconstituted BioGlo Luciferase Assay System (Promega G7941), and the resulting luminescence was quantitated using the Synergy HTX multi-mode reader (BioTek) and BioTek Gen 5 reader software. ADCC effector function was calculated by subtracting background signal from blank wells, averaging the triplicate reads of the PBS mock-digested, setting that ADCC value as 100% and determining the ratio of mock-digested ADCC compared to each sample's PNGase F or sialidase digested ADCC induction.

Lastly, to confirm that the exoglycosidase digestions did not impact the binding affinity of human IgG for VlsE antigen, plates were washed three times with 300 μL of 1×PBS (with 1 mM CaCl₂) and 0.1% Tween-20) and incubated with 100 μL of 0.05 ng/μL IRDye 680LT goat anti-human IgG (LiCor, 926-68078) diluted in 1×PBS at room temperature for 1 hour. Next, the plate contents were aspirated, washed three times with 300 μL of 1×PBS (with 1 mM CaCl₂) and 0.1% Tween-20) and imaged using a LiCor Odyssey CLx on the 700 and 800 IR laser channels with 84 μm scanning resolution set to medium quality with a 3.5 mm offset. The resulting data were quantitated using LiCor Image Studio.

### Antigen-Specific Complement Deposition Assay (ADCD)

Antibody-specific complement deposition against the VlsE antigen were assayed.

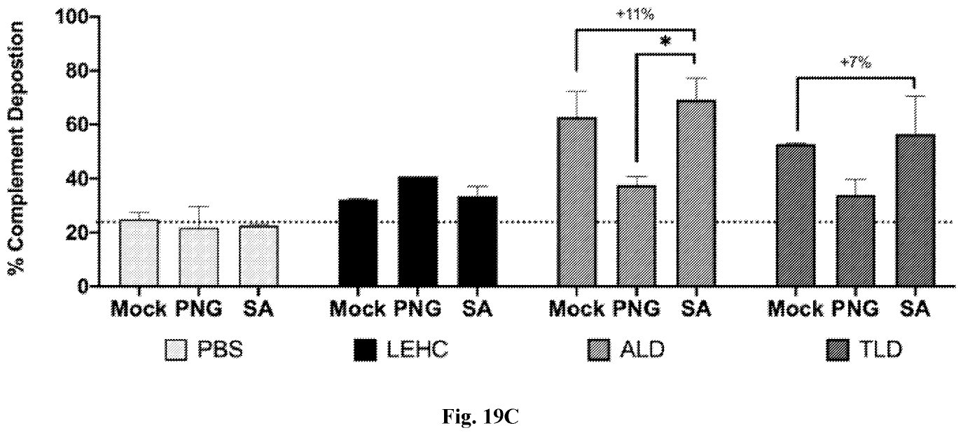

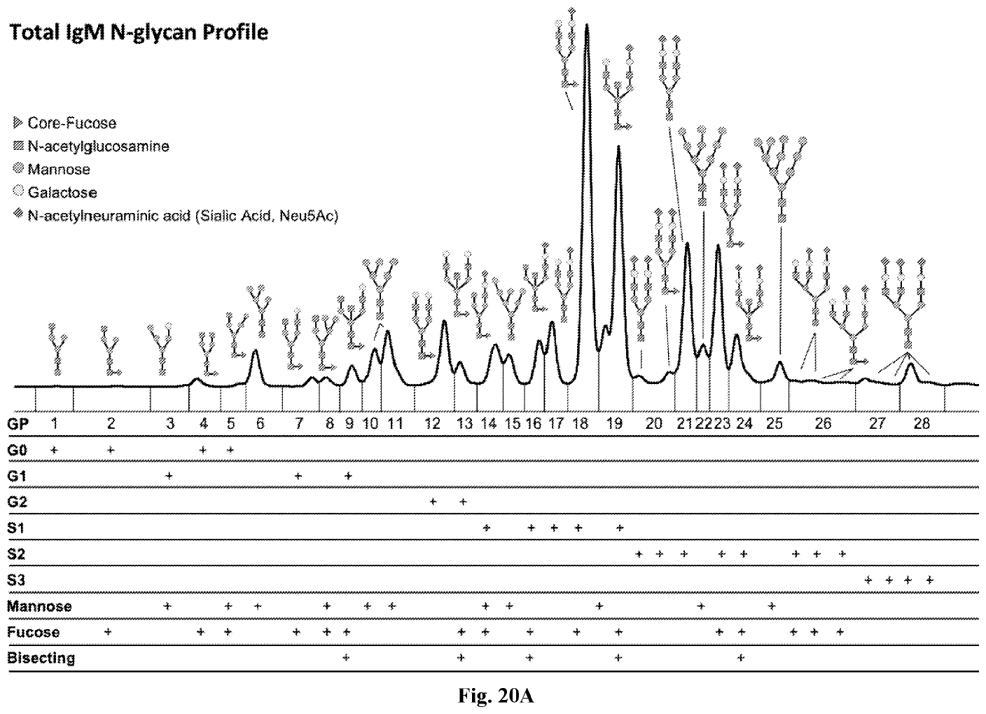

Briefly, 40 g of VIsE was biotinylated with a Micro Biotinylated Buffer with a subsequent desalting procedure (ThermoScientific, 1860301) as described by the manufacturer. Next, 40 μL FluoSpheres™ NeutrAvidin™-Labeled Microspheres (ThermoFisher) were incubated with 40 μg of biotinylated VlsE for 4 hours at 37° C. After washing twice with 200 μL 1×PBS, the antigen-bound beads were blocked with 200 μL 5% BSA in 1×PBS for 1 hour at 37° C. Next, the beads were washed twice with 500 μL of 0.1% BSA in 1×PBS and diluted 1:100 in 1×PBS. Next, 25 μL of the 1:100 bead solution was transferred to low-binding 1.5 mL tubes (Costar, 3207) and incubated with 20 μL of 1:10 1×PBS diluted purified, and pooled IgG at 4° C. overnight. Of note, the IgG was purified from sera using the protein method listed above. Next, the immune-complexed beads were incubated at 37° C. for 15 minutes with freshly resuspended Guinea pig complement (Cedarlane, CL4051) diluted 1:50 in Gelatin Veronal Buffer with Mg2+ & Ca²⁺ (GVB++). The complement deposition was halted with two washes of ice-chilled 200 μL 15 mM EDTA. Next, 50 μL of a 1:100 diluted FITC labeled Goat anti-Guinea pig Complement C3 antibody (MP Biomedicals, 085538) was incubated with the immune-complexed beads at 37° C. for 15 minutes. Lastly, two 200 μL 1×PBS washes removed unbound FITC labeled anti-C3 antibody. Washed samples were re-suspended in 200 μL 1×PBS and analyzed using a Fortessa Flow Cytometer (BD). Beads were gated for the presence or absence of the FITC antibody, and the MFI of the bead content was divided by the total number of beads to determine the rate of complement deposition in each sample. Patient pools were incubated with FITC antibody without the presence of complement to establish background fluorescence for the assay. Patient values were adjusted by subtracting the background mean fluorescence for each sample pool. The gating strategy is displayed in FIG. 19A.

### Statistical Analysis

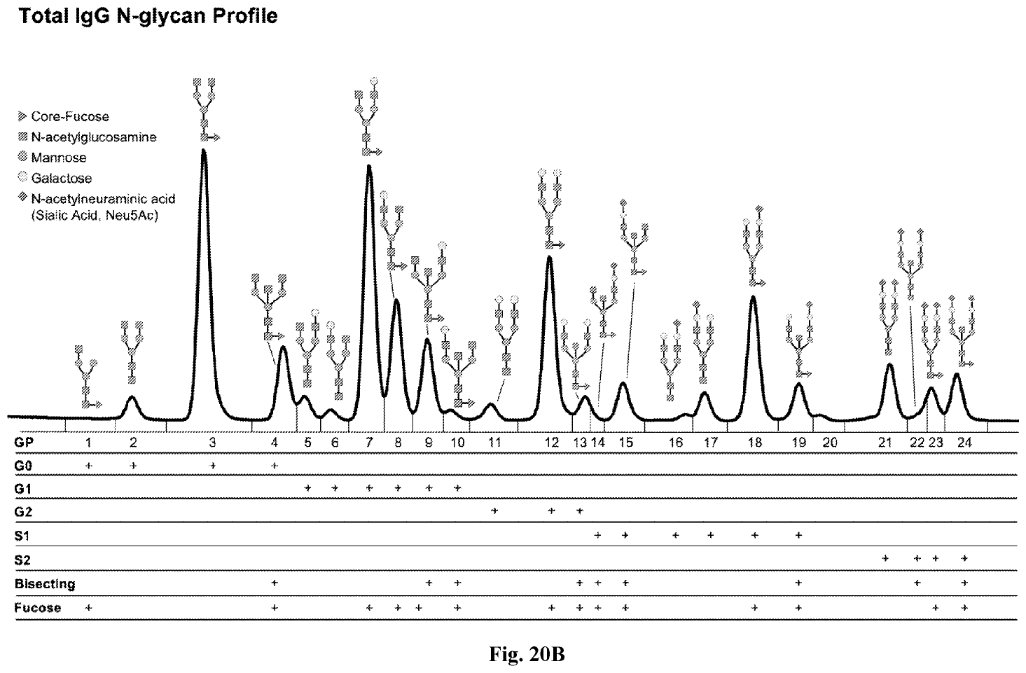

Raw N-glycans from total IgG and total IgM UPLC quantitation includes the relative abundance of 24 IgG N-glycans, 28 IgM N-glycans, and IgG and IgM N-glycan classes summarized from IgG and IgM N-glycans. IgG Nglycan classes include G0, G1, G2, S1, S2, bisecting, fucosylated, total G1, and total G2. IgM N-glycan classes include G0, G1, G2, S1, S2, S3, mannose, bisecting, fucosylated, and hybrids. See FIGS. 20A-20B for a detailed layout of peak identification and N-glycan grouping. Log-ratio transformation was performed on relative abundance values of all pairwise combinations of IgG N-glycans and all pairwise combinations of IgM N-glycans. Mean and standard deviations were calculated for each relative abundance of glycans and glycan class within each diagnostic group, and percent change in each glycan relative abundance between matched ALD and TLD were calculated by (TLD-ALD)/ALD×100 for each patient and then averaged to obtain mean percent change.

Nested stratified 10-fold cross-validation was performed for all machine learning methods including support vector machine (SVM), naïve bayes (NB), least absolute shrinkage and selection operator (lasso)+NB, lasso+SVM, lasso+logistic regression, elastic net, random forest (RF), gradient boosting machine (GBM), and extreme gradient boosting trees (XGBOOST). The predicted probability obtained from each test fold was aggregated to produce receiver operating characteristic (ROC) curves and Area under the ROC Curve (AUC) values.

### Hyperparameters Were

selected using grid search and 10-fold cross-validation within each training cohort. Hyperparameters for each algorithm (if applicable): (a) SVM: gamma=2\hat{ }(−8, −7, −6, −5, −4), cost=1,2,3,4, and 5, epsilon=0.05 (b) RF: Number of trees=60000, mtry=square root of total number of predictors rounded to the lower integer (c) GBM: Number of trees=60000, shrinkage=0.001, maximum depth of each tree=5 (d) XGBOOST: Max number of boosting iterations=300 and 500. Max tree depth=4 and 6. Shrinkage (eta)=0.01, 0.05, and 0.1, Minimum loss reduction (gamma)=0.01 and 0.05, Subsample Ratio of Columns=0.1 and 0.25. Subsample Percentage=0.5 and 0.75. (e) elastic net: alpha=0, 0.1, 0.2, . . . , 0.8, 0.9, 1.

For each algorithm, raw N-glycan data and log-ratios combined with raw data were used to produce and compare AUC values with and without log-ratio transformation. N-glycans from IgG, IgM, or a combination of IgM and IgG were used to train and cross-validate the discrimination between cohorts, resulting in the ROC curves. Paired Delong's test was used to test the difference between two ROC curves on the same patients, and the unpaired Delong's test was used to test the difference between two ROC curves on different patients. One-way ANOVA with Tukey's multiple comparisons test was used to compare GlycoLyme score and IgG abundance signal among multiple diagnostic groups. Paired t-tests were used to compare IgG abundance or SNA fluorescence between matching ALD and TLD patients. Nonparametric repeated measures one-way ANOVA with Dunn's multiple comparisons post-hoc test was used to compare anti-VlsE IgG abundance and Normalized VlsE ADCC, and normalized VlsE ADCD among mock-exoglycosidase, PNGase F, and sialidase measured on the same patient cohorts. A p-value of <0.05 was considered statistically significant for all tests. The statistical software R version 4.2.3 and GraphPad Prism 8 were used to analyze the data.

### Example 4-3: IgG and IgM N-Glycans Discriminate Acute Lyme Disease from Patients Treated for Lyme Disease and Endemic Controls

The present study sought to determine how total IgG and total IgM N-glycomes from acute Lyme disease (ALD) patients differed from Lyme-endemic healthy control (LEHC), non-Lyme-endemic healthy controls (NLEHC), treated Lyme disease (TLD) at a 2-3 month convalescent draw, lupus (L), rheumatoid arthritis (RA), fibromyalgia (F), and syphilis (S). Patient sera samples were obtained from the Lyme Disease Biobank (LDB) and the Precision for Medicine Biobank (PFM). Patient demographics are listed in FIG. 15 including cohort size, average age, sex, race, and presence of erythema migrans (EM) as well as positive serologic testing results from IgM and IgG western immunoblots, C6 peptide ELISA, and the standard two-tiered test (STTT). Of note, samples from ALD and LEHC patients tested PCRnegative for other tick-borne infections including Anaplasma phagocytophilum, Babesia microti, and Ehrlichia chaffeensis.

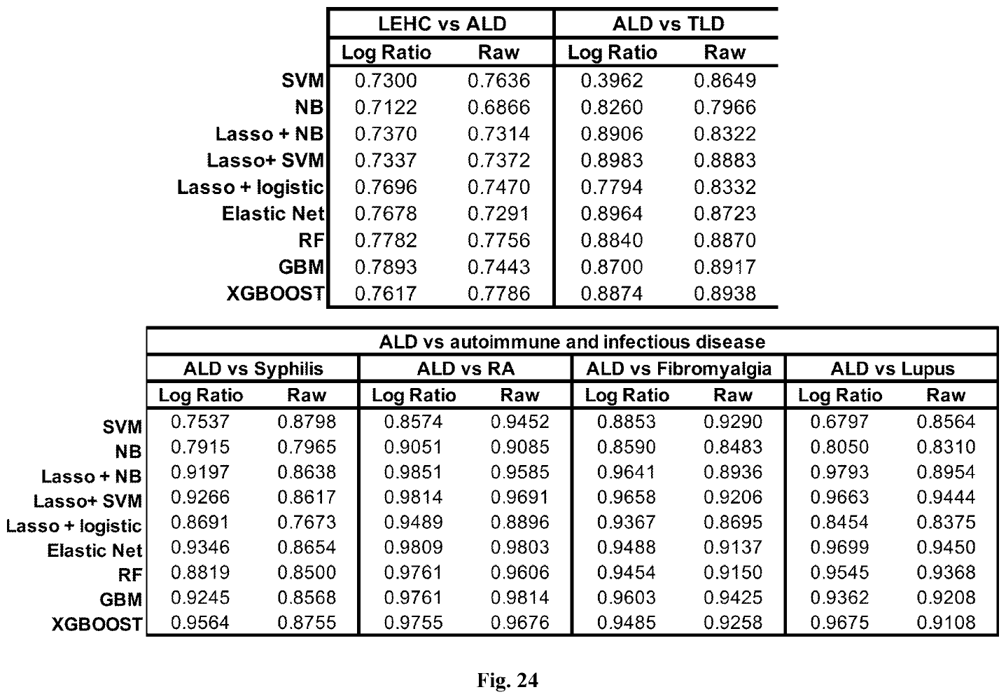

Total IgG and total IgM were purified from each patient using affinity chromatography. Next, N-glycans from each immunoglobulin were released, labeled, quantitated, and identified using a UPLC-FLR-ESI-MS approach. This resulted in 28 IgM N-glycan species and 24 IgG N-glycan species, detailed in FIGS. 20A-20B. Furthermore, IgG N-glycans were grouped into 9 characteristic classes depending on the galactose, sialic acid, and core-fucose characteristics. A similar grouping of IgM N-glycans resulted in 10 IgM N-glycan classes. This yielded 71 ‘raw’ Nglycan features. These 71 raw N-glycans features were log-ratio transformed to create an additional 1,231 features. Raw N-glycan features and log-ratio transformed N-glycan feature sets were analyzed using the lasso, naïve bayes, support vector machine, random forest, gradient boosting machine, or extreme gradient boosting trees machine learning methods. Comparisons of the area under the curve (AUC) are listed in FIG. 24. The present study found the random forest (RF) method trained on raw N-glycan features performed the best across all machine learning methods independent of log-ratio transformation overall. Therefore, the present study presents the receiver operating characteristic (ROC) curves of RF cross-validation to compare ALD N-glycans to other cohorts.

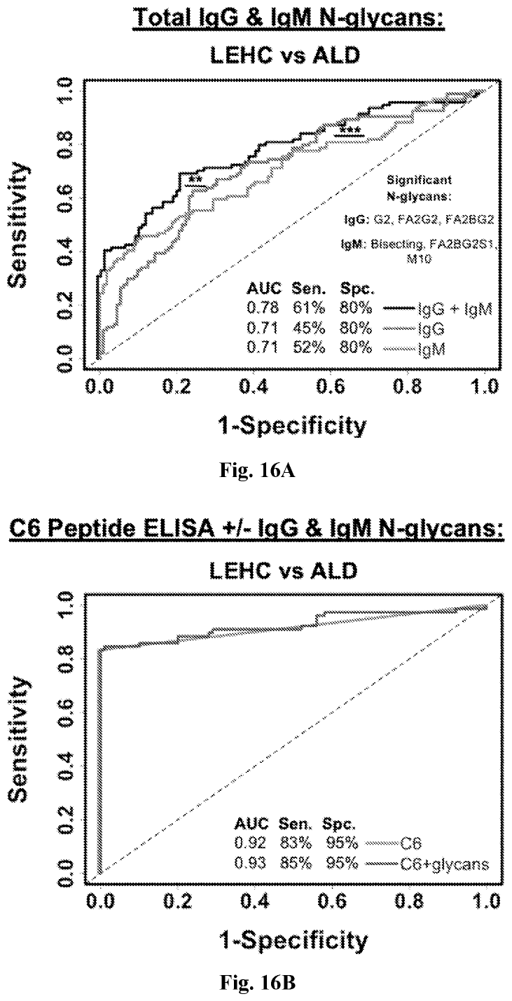

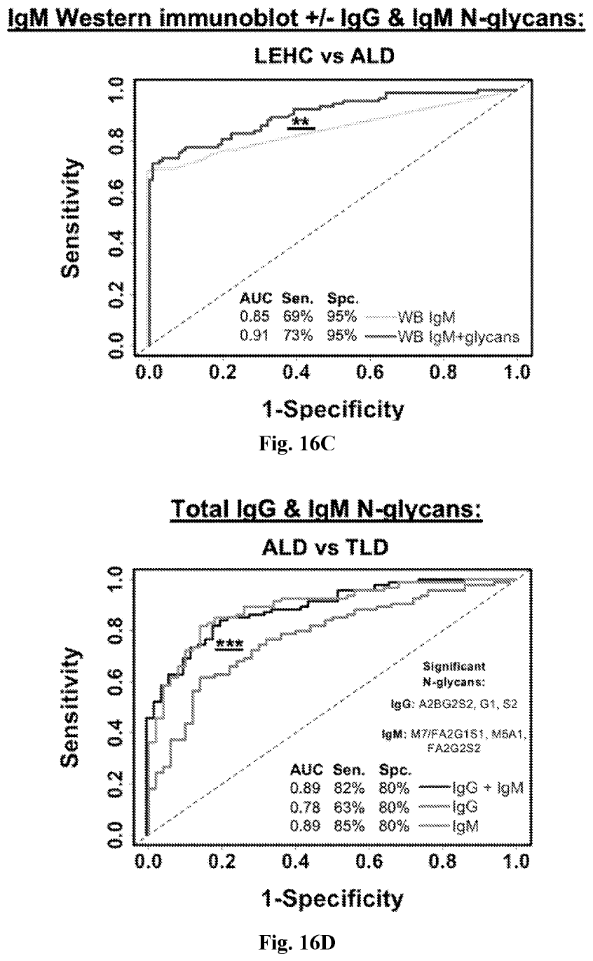

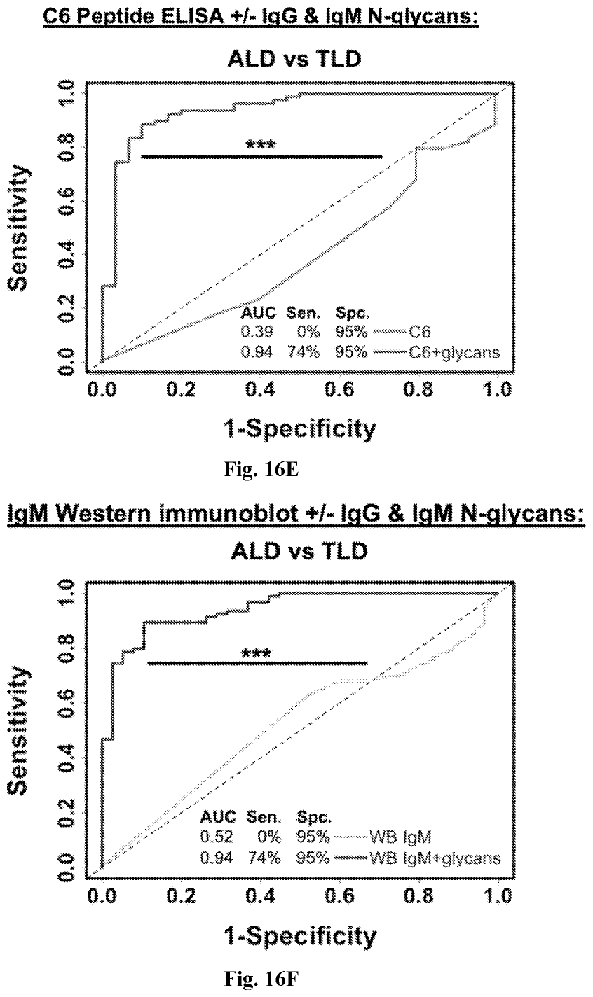

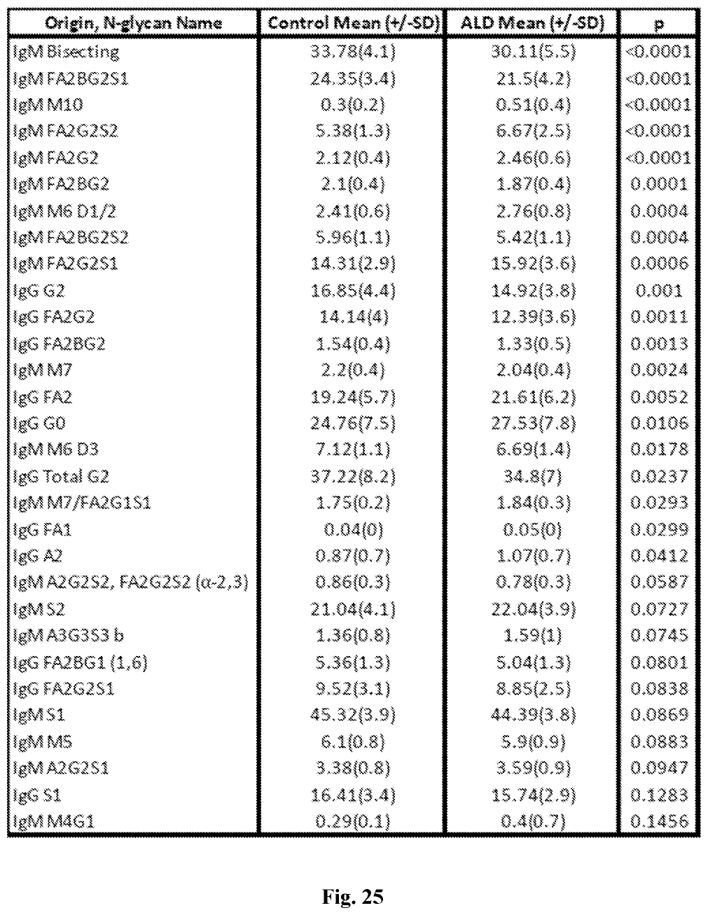

The present study compared IgG and IgM N-glycans from LEHC and ALD patient cohorts (FIGS. 16A-16C). The random forest machine learning model discriminated between LEHC and ALD patient cohorts with an AUC of 0.78 using a combination of IgG and IgM N-glycans (FIG. 16A). Important N-glycans were ranked by the student's t-test p-values, identifying di-galactosylated IgG N-glycans and the IgM N-glycans: bisecting class, FA2BG2S1, and M10. A complete list of the most significant N-glycans is included in FIG. 25. Interestingly, IgG N-glycans exhibited lower cross-validation performance compared to IgM N-glycans as calculated using DeLong's test (FIG. 16A). The present study next sought to determine if the combination of the IgG and IgM N-glycomes could improve the diagnostic accuracy of the C6 peptide ELISA assay performed on the LEHC and ALD cohorts (FIG. 16B). There, the present study observed the AUC increased from 0.92 to 0.93 when IgG and IgM N-glycans were included. The present study observed a greater improvement in the Lyme disease IgM western immunoblot performance when N-glycans from IgG and IgM were included, increasing the AUC from 0.85 to 0.91 (FIG. 16C). This difference was statistically significant between ROC curves after applying paired DeLong's comparison test (p<0.004). Taken together, we determined that IgG and IgM N-glycans can discriminate between ALD and LEHC, and in some cases can improve diagnostic accuracy such as the Lyme disease IgM western immunoblot.

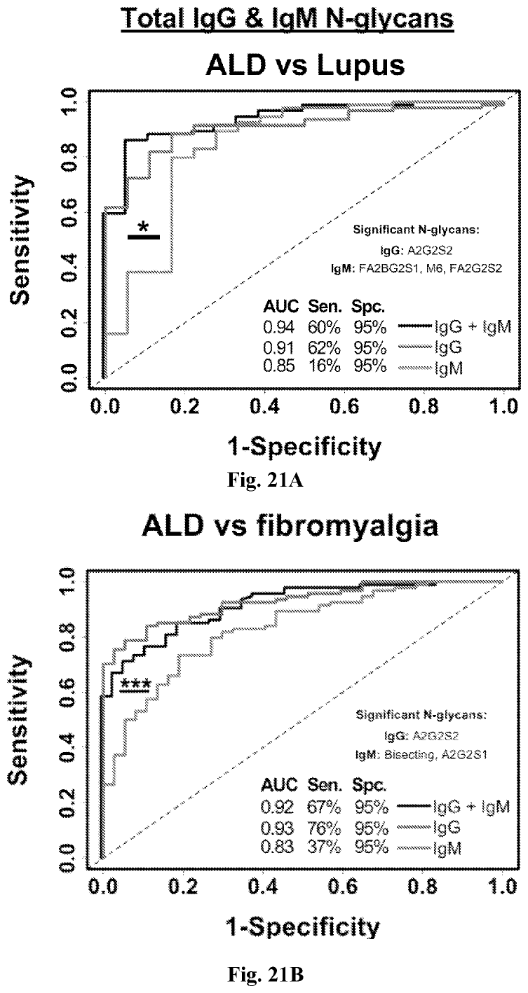

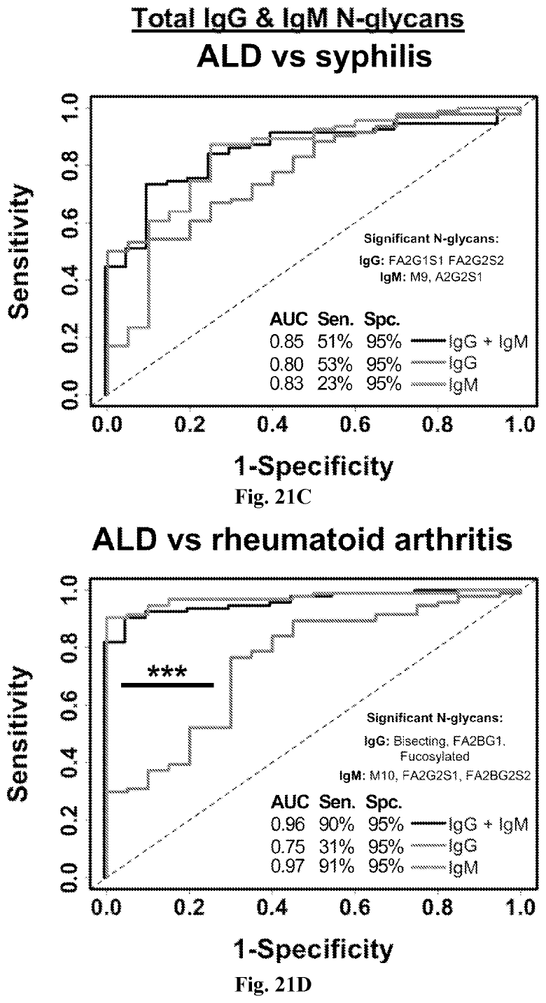

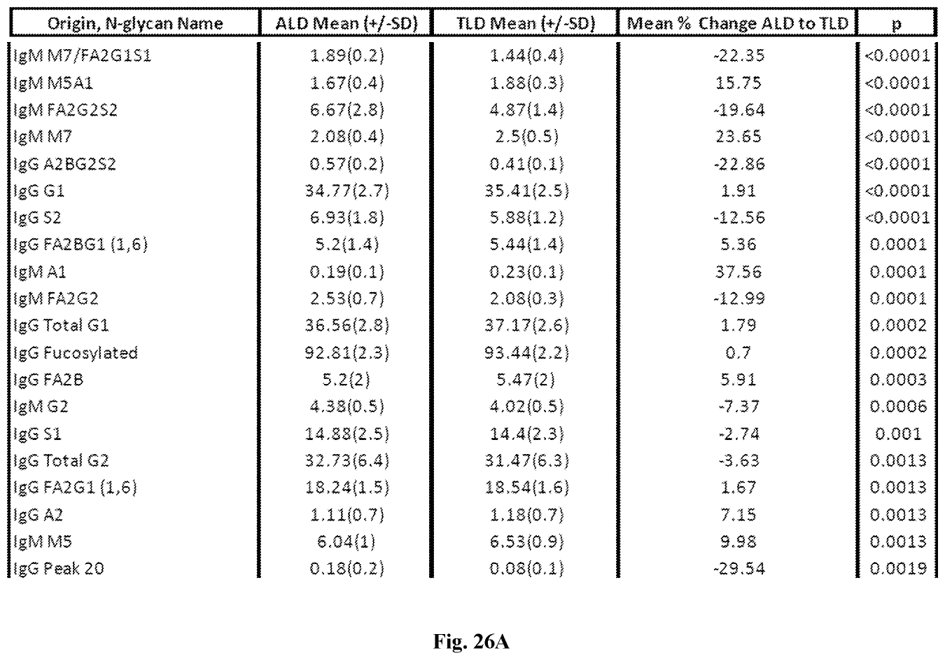

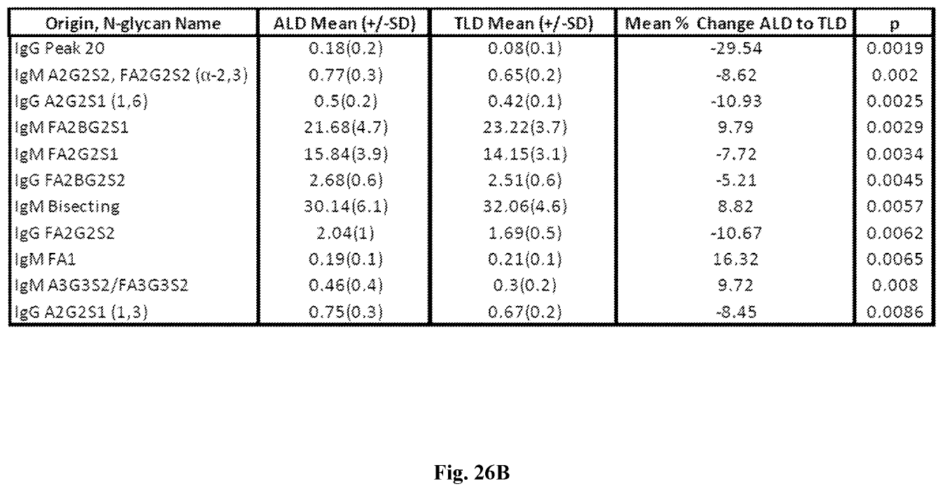

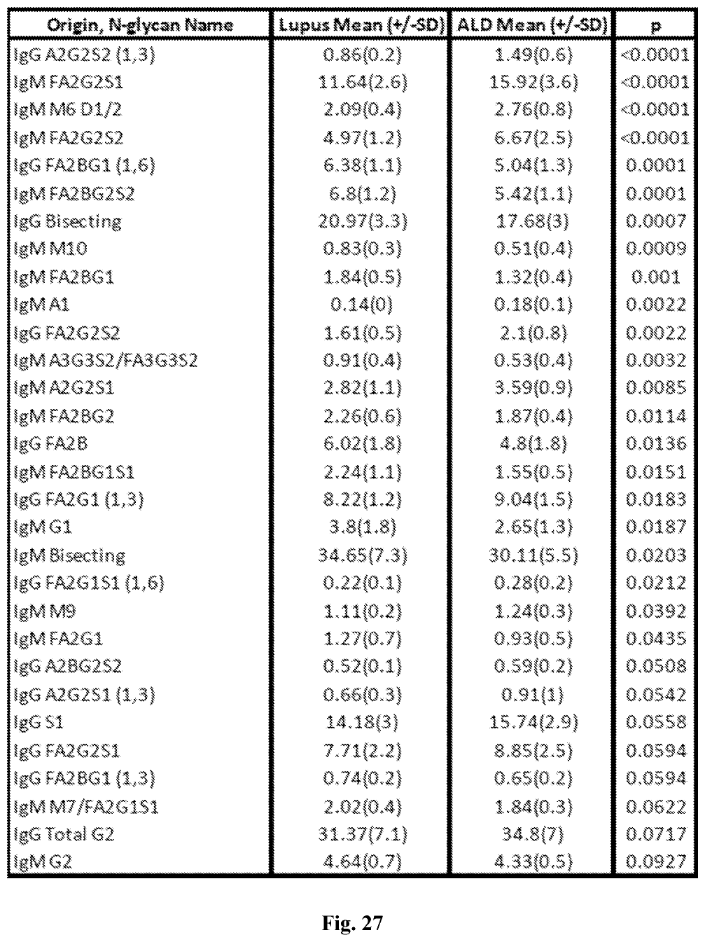

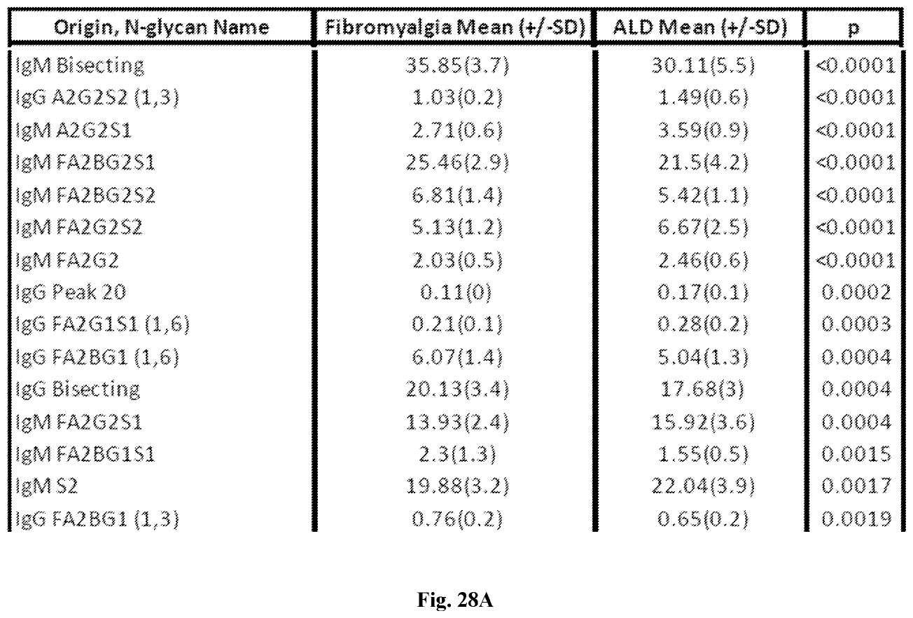

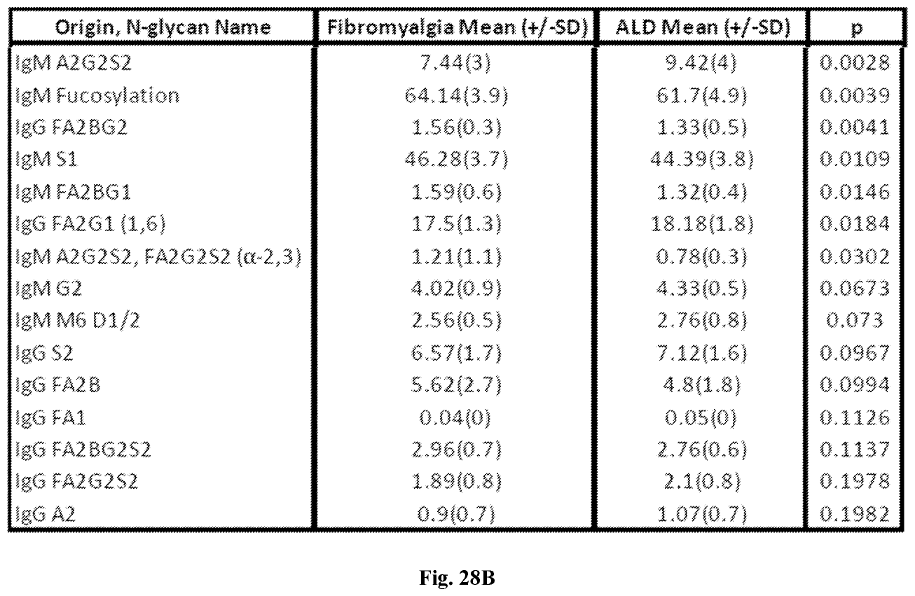

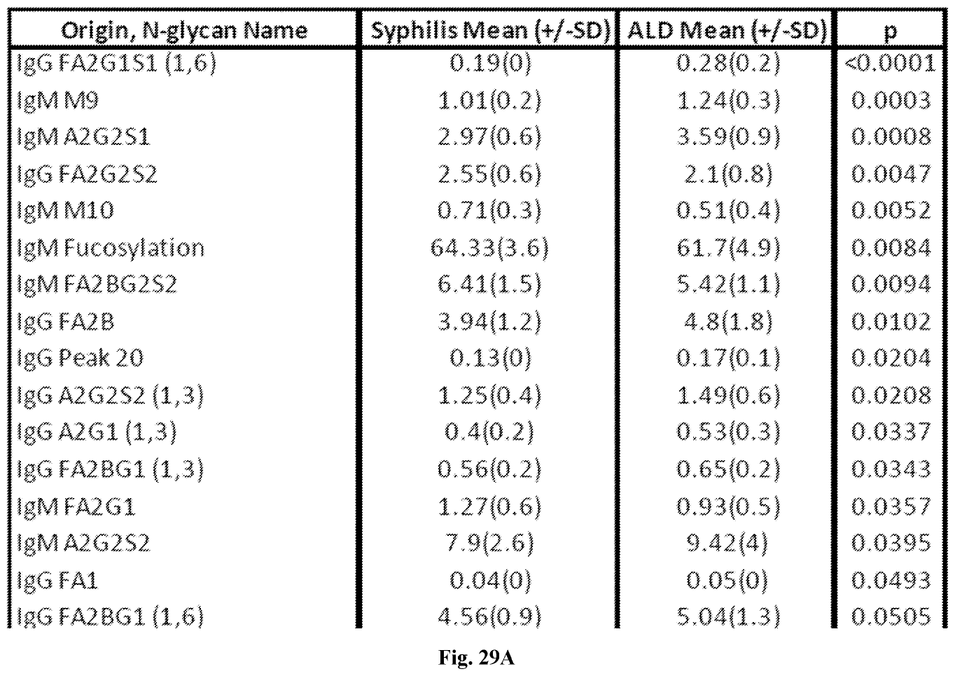

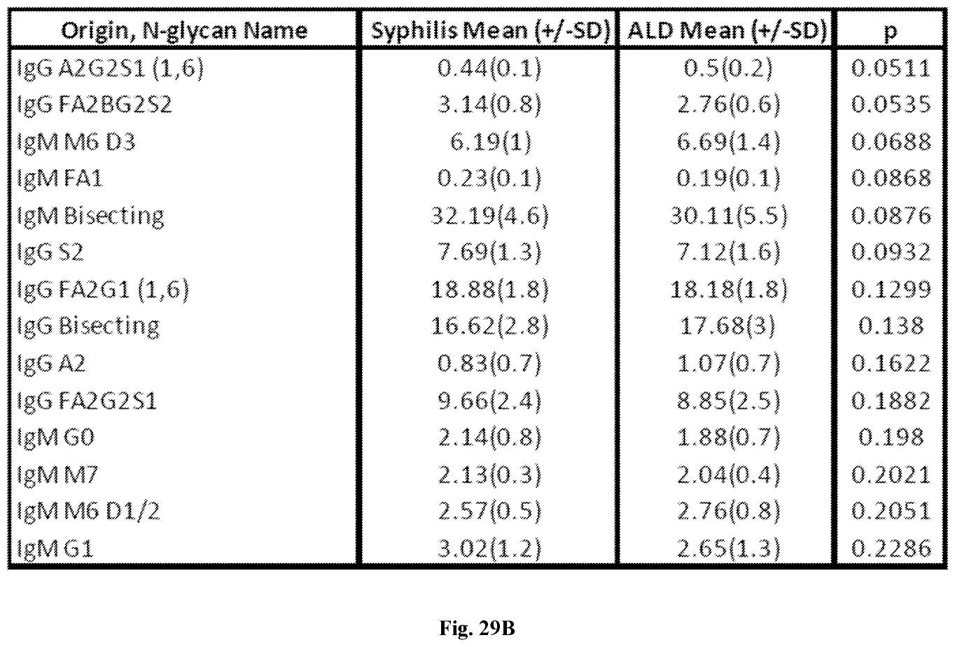

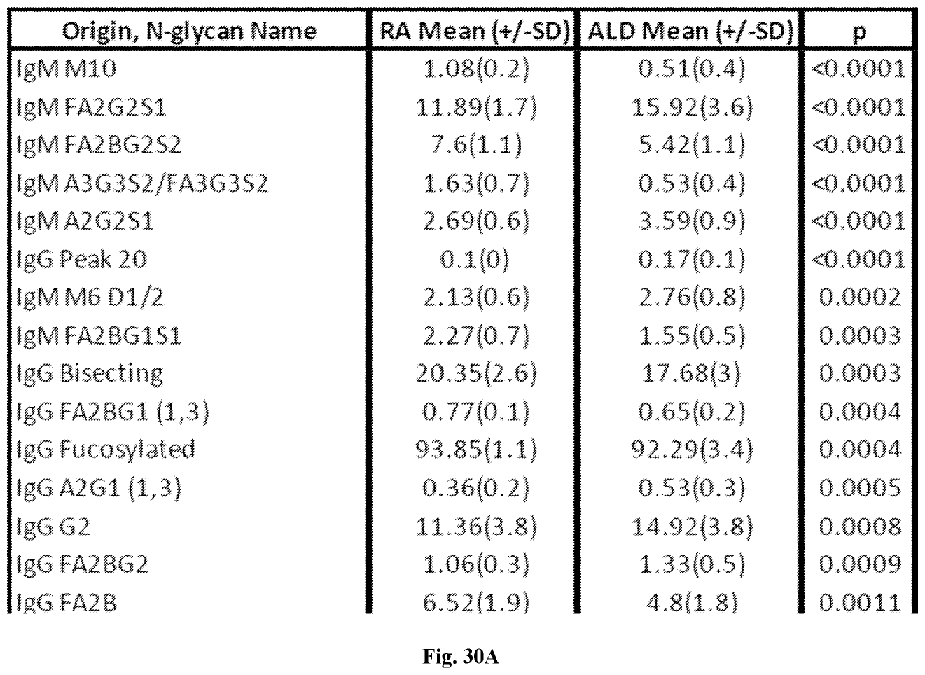

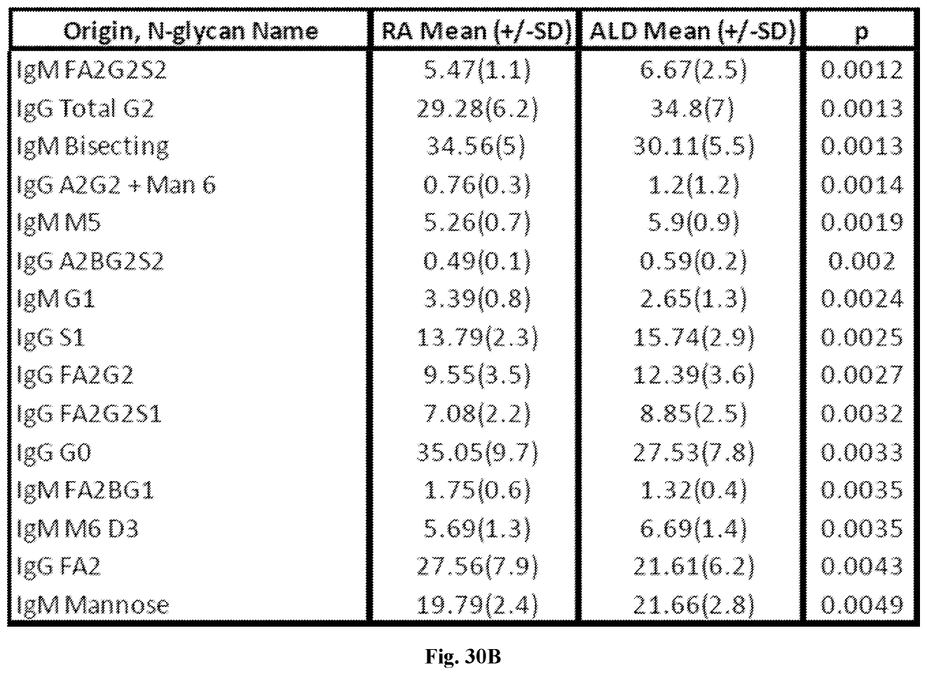

Next, the present study compared the ALD cohort to patients diagnosed with acute Lyme disease that received antibiotics at the time of diagnosis and then donated additional sera after completing a 2-3 month convalescence period (FIGS. 16D-16D). The present study refer to this cohort as “treated Lyme disease” (TLD). This comparison of ALD to TLD is important because current serologic assays cannot discriminate ALD patients from TLD patients who have responded to antibiotic treatment following diagnosis with acute Lyme disease. Further, 90% of the TLD cohort reported that the initial antibiotic therapy prescribed following diagnosis with acute Lyme disease resolved their symptoms. Total serum IgG and IgM N-glycans discriminated ALD versus TLD with higher accuracy than ALD versus LEHC (FIG. 16D). The random forest model obtained an AUC of 0.89 with a sensitivity of 82% and a specificity of 80% on cross-validation between ALD and TLD using total IgG and IgM N-glycans. Interestingly, the most significant N-glycan predictors included the di-sialylated IgG A2BG2S2 and IgM FA2G2S2 when analyzing paired ALD and TLD samples (FIGS. 26A-26B). Further, this trend of ALD immunoglobulins containing higher levels of sialic acid and galactose was maintained when comparing ALD to lupus, fibromyalgia, rheumatoid arthritis, and syphilis (FIGS. 27, 28A-28B, 29A-29B, and 30A-30B). In the comparison of ALD to other diseases of similar etiology, the combination of total IgG and total IgM N-glycans yielded AUCs ranging from 0.85-0.96 (FIGS. 21A-21D).

Finally, the present study determined if the combination of IgG and IgM N-glycans could be used in conjunction with the C6 peptide ELISA to improve the accuracy of discrimination between ALD and TLD (FIG. 16E). The present study observed a significant improvement in the random forest model to discriminate between ALD and TLD when incorporating total IgG and IgM N-glycans, increasing from an AUC of 0.39 to 0.94, with a sensitivity of 74% and a specificity of 95% on cross-validation (p<0.001). Because IgM responses are generally limited to the first 30 days of acute immune responses, we assessed the ability of the Lyme disease IgM western immunoblot to discriminate between the ALD and TLD cohorts (FIG. 16F). The Lyme disease IgM western immunoblot alone exhibited poor discrimination, with 0% sensitivity at 95% specificity. When the total IgG and IgM N-glycome were incorporated with the Lyme disease IgM western immunoblot data, the AUC improved from 0.52 to 0.94 on cross-validation (p<0.001) with 74% sensitivity and 95% specificity. Taken together, total IgG and total IgM N-glycomes provide potential biomarkers that specifically identify ALD cohorts compared to TLD cohorts.

### Examples 4-4: Development of Bb-Specific Lectin Multiplex Assay

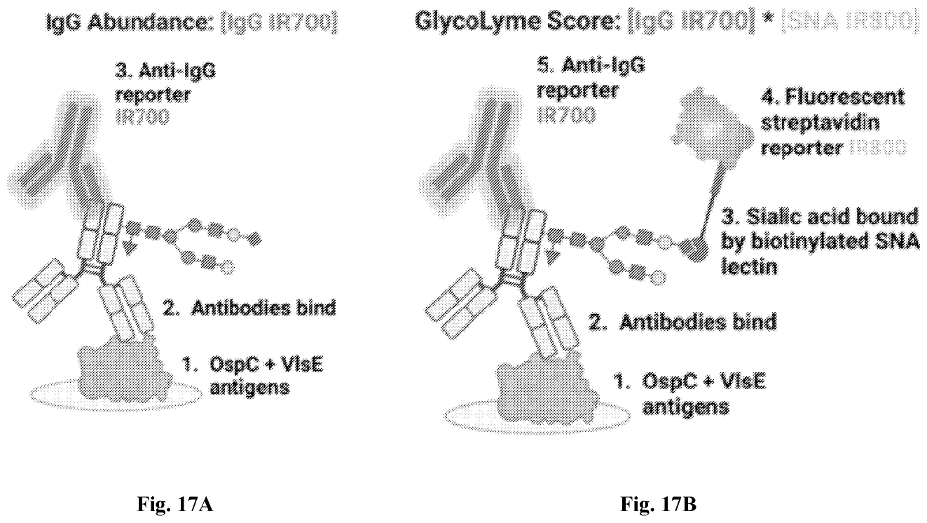

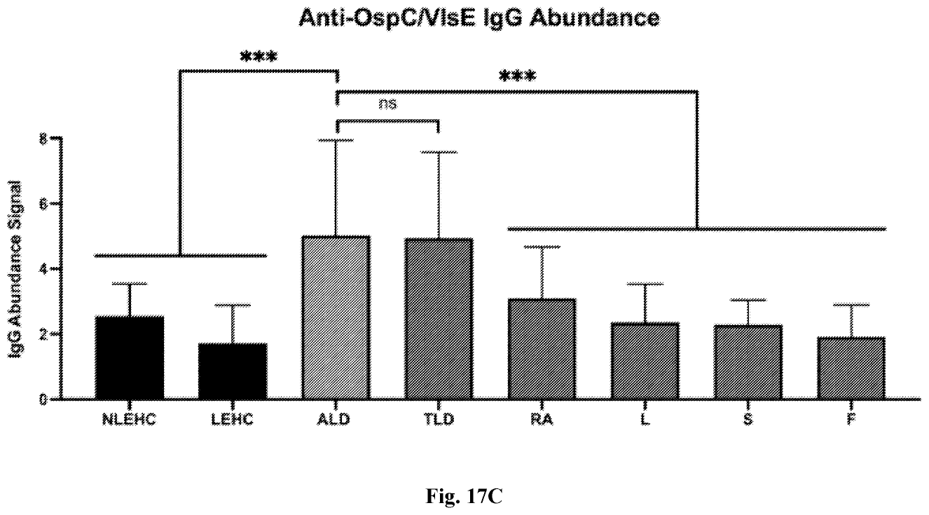

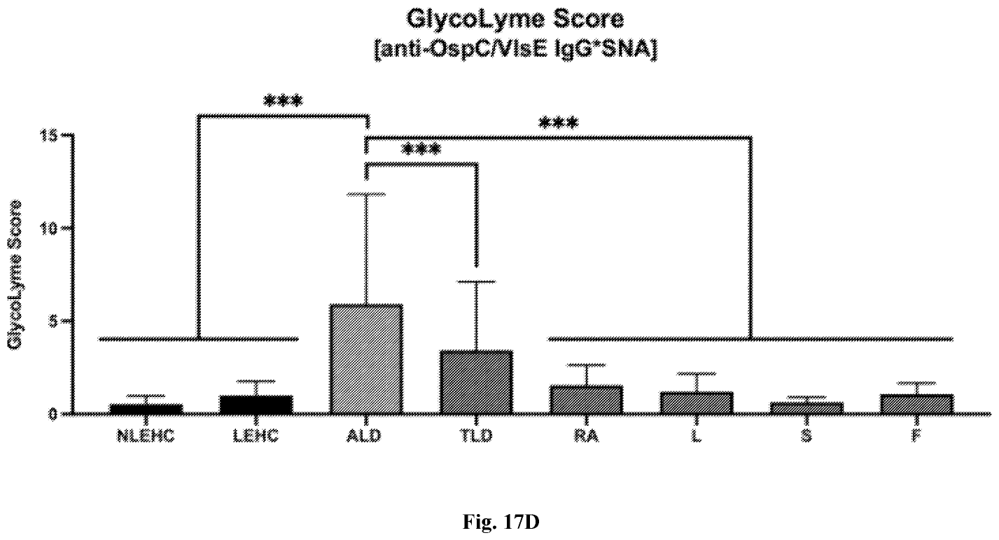

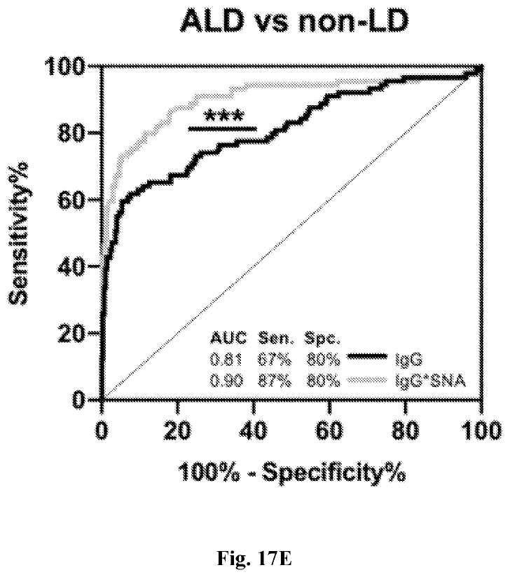

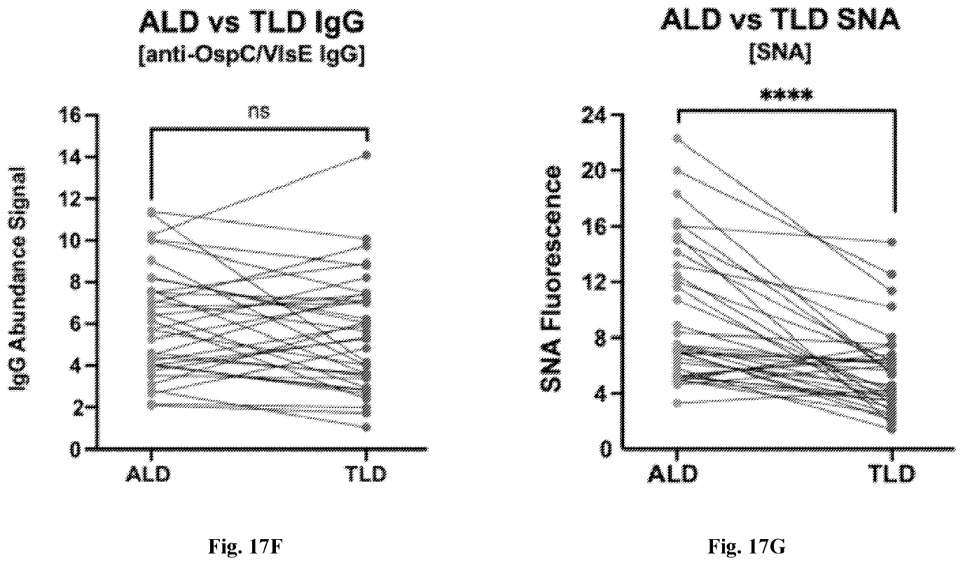

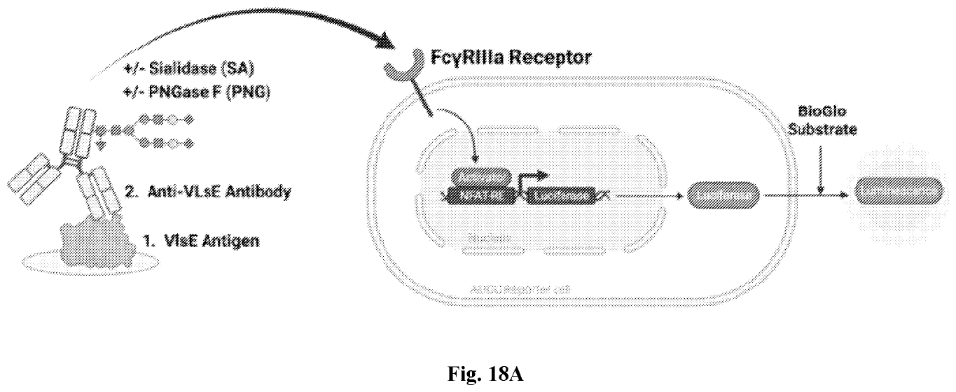

With the detection of increased sialic acid on total IgG and total IgM of acute Lyme disease patients, the present study developed a plate-based lectin multiplex assay to interrogate the sialylation of Bb-specific immunoglobulins of ALD and TLD cohorts. The present study began by optimizing a fluorescent detection method to quantitate anti-Bb IgG (FIG. 17A), then the present study compared the performance of the assay when accounting for the abundance of sialic acid on the isolated anti-Bb immunoglobulins (FIG. 17B). This plate-based lectin multiplex assay first isolates antibodies reactive to recombinant Outer Surface Protein C (OspC) and Variable Lipoprotein Surface-Exposed (VlsE) protein. The combination of OspC and VlsE antigens yielded the highest AUC during pilot experiments (FIG. 22). Next, the biotinylated lectin Sambucus Nigra (SNA) was incubated to detect sialic acid content, reported by a streptavidin-conjugated fluorophore. Lastly, an anti-IgG reporter conjugated to a different fluorophore was incubated to quantitate the amount of IgG bound (FIG. 17B). This stepwise approach starting with SNA ensures that only the glycans associated with patient immunoglobulins were quantified because OspC and VlsE do not contain sialic acid and the anti-IgG reporter is not present during SNA incubation. A final GlycoLyme score was determined by multiplying the amount of sialic acid detected by the amount of anti-OspC/VlsE IgG detected.

In comparison to the traditional quantitation of Bb-specific IgG (FIG. 17C), the quantitation of Bb-specific IgG and sialic acid abundance improved the detection of the ALD cohort (FIG. 17D). As expected, the ALD cohort was discriminated from Lyme endemic healthy controls (LEHC), non-Lyme endemic healthy controls (NLEHC), rheumatoid arthritis (RA), lupus (L), syphilis (S), and fibromyalgia (F) cohorts in both the anti-OspC/VlsE IgG-only and the GlycoLyme score approach. Yet the GlycoLyme approach improved the AUC from 0.81 to 0.90 and the sensitivity from 67% to 87% while maintaining specificity (FIG. 17E). In addition, by accounting for the abundance of sialic acid, the GlycoLyme score discriminates TLD patients from ALD patients in a statistically significant manner (p<0.001) while anti-OspC/VlsE alone did not. Therefore, the inclusion of sialic acid quantitation significantly improves the current Lyme disease diagnostic-specifically for the acute stage.

The present study next sought to interrogate the relationship of sialic acid on antigen-specific immunoglobulin populations during ALD compared to TLD. The present study analyzed the concentration of anti-OspC/VlsE IgG from the ALD cohort with the matching TLD cohorts that met the GlycoLyme score threshold of >2 (70% of matching ALD and TLD pairs). There, the present study did not observe statistically significant differences between ALD and TLD IgG abundance (FIG. 17F). On the other hand, examining the relative abundance of sialic acid detected using the GlycoLyme method led to a statistically significant reduction of SNA fluorescence (p<0.0001) at the matching TLD time point (FIG. 17G). This suggests that following antibiotic therapy and a 2-3 month convalescence period, the glycosylation of anti-Bb immunoglobulins begins to return to a baseline. The mean reduction in sialic acid in convalescence sera was 27%.

Next, the present study analyzed the complete ALD and TLD GlycoLyme scores by biological sex. It was indicated that Lyme disease symptoms were higher in female. The present study reports that 65% of ALD females lost between 1.1- to 11-fold of the GlycoLyme score at the TLD time point, while 35% of the female ALD GlycoLyme scores did not resolve at the TLD time point. TLD females with unresolved GlycoLyme scores did not correlate to age, days between ALD to TLD sample donation, or patients' reports of ongoing symptoms following antibiotic therapy. Yet, there was a significant positive correlation (p<0.03) between TLD females with a previous history of Lyme disease and a lack of decrease from the ALD to TLD GlycoLyme scores. In comparison, 73% of ALD males lost between 1.2- to 8-fold of the GlycoLyme score at the TLD time point while the remaining 26% of the male GlycoLyme scores were unresolved at the TLD time point. Correlations between male TLD GlycoLyme score and age, days between ALD to TLD sample donation, patient's reports of ongoing symptoms following antibiotic therapy, or a previous history of Lyme disease were statistically insignificant. Further investigation with matching cohorts of ALD and TLD patients collected across multiple time points is required to determine what the decrease in GlycoLyme score signifies in the TLD population. For now, these data suggest that immunoglobulins responding to ALD are highly sialylated and that this sialylation begins to return to a healthy baseline following successful antibiotic treatment and months of convalescence. Thus, the present study identifies a novel potential biomarker of acute Lyme disease.

### Example 4-5: Sialic Acid Content on Bb-Specific IgG Impairs Antibody-Dependent Cellular Cytotoxicity

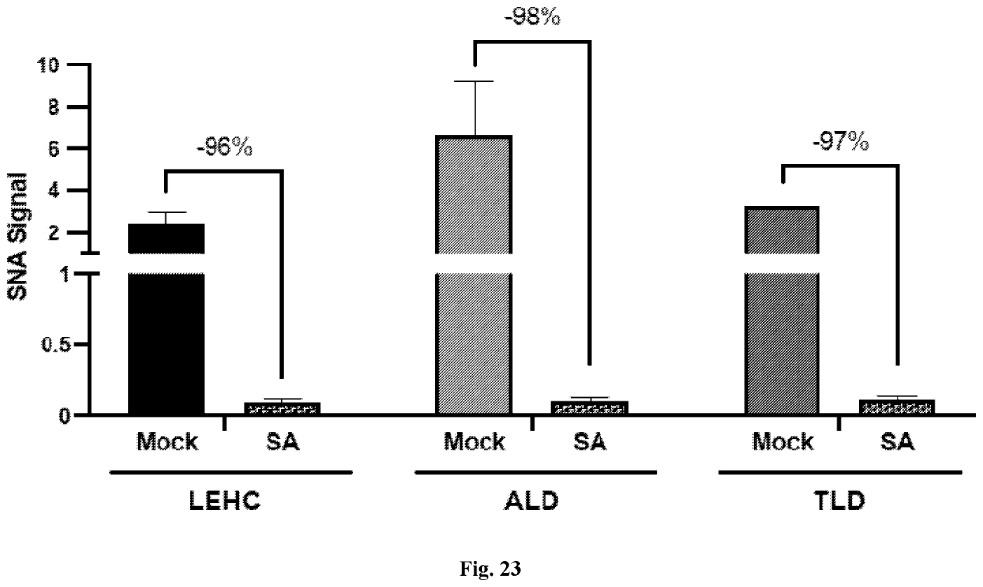

Increased sialic acid content on core-fucosylated IgG modestly reduces the induction of antibody-dependent cellular cytotoxicity (ADCC) in other disease states. The present study designed an experiment to determine how the sialic acid on ALD anti-VlsE IgG impacts ADCC induction by modifying the N-glycan structures enzymatically. Due to the volume of sera required for the functional immunology assays, sera from the LEHC, ALD, and TLD cohorts were pooled into two, four, and two sample sets respectively. Next, an aliquot of each pooled serum set was digested with a sialidase (SA) to remove sialic acid from the IgG Fc region. A second aliquot of each pooled serum set was digested with PNGase F (PNG) to remove the entire N-glycan from the IgG Fc region as a negative control. Lastly, a third aliquot of each pooled serum set was mock-digested (Mock) with 1×PBS to control for the 12-hour 37° C. glycandigestion procedure. Sialidase digestions resulted in anti-VlsE antibodies retaining less than 4% of the SNA reactivity compared to the mock-digested cohort FIG. 23.

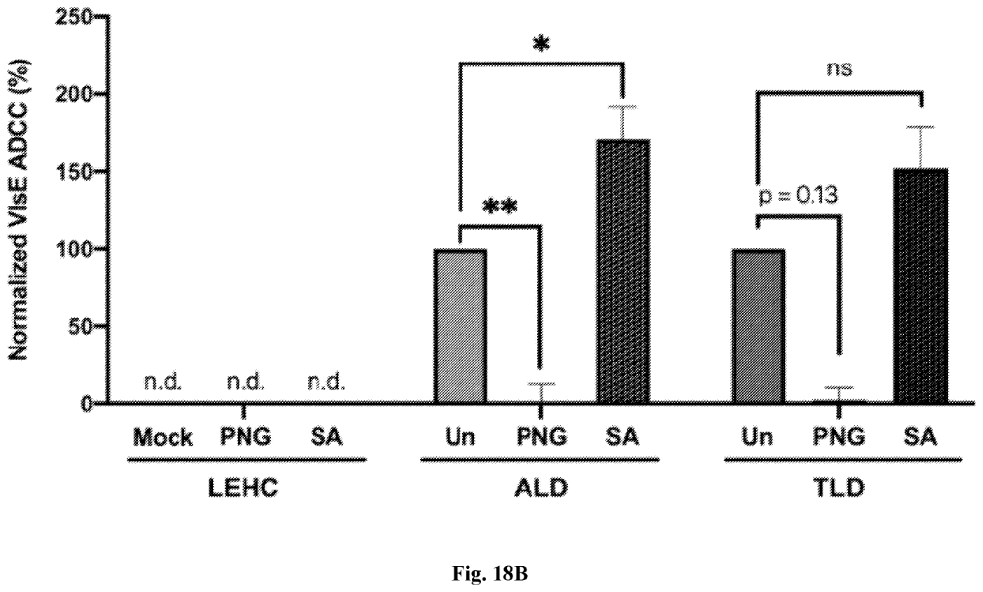

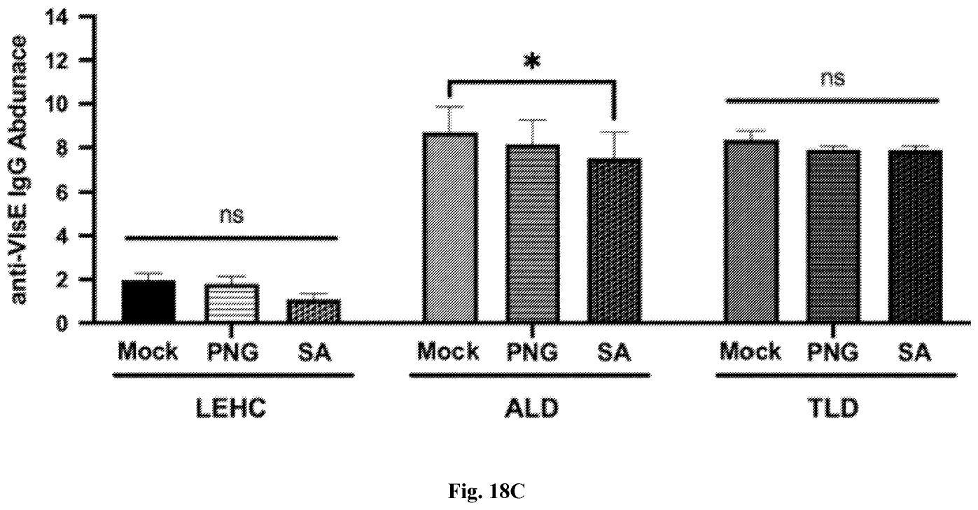

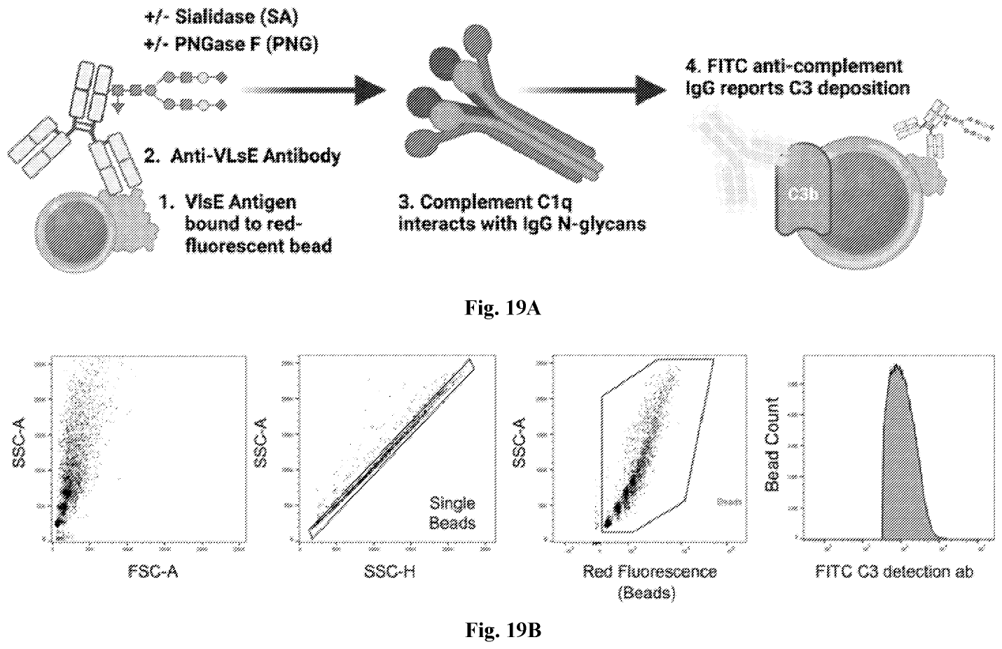

Next, the mock (Mock), sialidase (SA), or PNGase F (PNG) digested sera sets were incubated in triplicate on plates coated with the VlsE antigen. Then the bound anti-VlsE IgG was assayed for the ability to induce ADCC using an engineered Jurkat cell line expressing firefly luciferase if the FcγRIIIa receptor was activated (FIG. 18A). The LEHC antibodies did not induce detectable ADCC, as anticipated (FIG. 18B). The ALD cohort ADCC induction was detected in the mock-digested set (Mock) and the ADCC activity was abolished following PNGase F (PNG) mediated removal of the IgG N-glycan. The requirement of the intact N-glycan on IgG for ADCC induction has been demonstrated previously and was expected as a negative control in this experiment Wada et al. (mAbs, 2019. 11(2):p. 350-372).

Following the removal of IgG sialic acid from ALD anti-VlsE IgG, the induction of ADCC significantly improved by over 70% compared to the mock-digested ALD IgG. To control for the impact of exoglycosidase digestion on antibody affinity for the VlsE antigen, the plate-bound anti-VlsE IgG assayed for ADCC induction was subsequently quantitated (FIG. 18C). Across the LEHC, ALD, and TLD cohorts, there were similar levels of anti-VlsE IgG, independent of Nglycan digestion method with one exception. ALD anti-VlsE IgG digested with SA led to a 12% reduction of antibody abundance. The reduced anti-VlsE IgG content following SA-digestion could suggest a decrease in IgG Fab affinity for VlsE. This is because the Fab light chains of IgG have been noted to contain sialylated N-glycans, but at much lower levels than the Fc heavy chain of IgG. Regardless, while there were on average lower levels of anti-VlsE IgG per SA-digested ALD ADCC assay, we still observed a significant increase in the relative amount of cellular cytotoxicity induced. In contrast, the ALD PNG-digested samples demonstrate that IgG affinity is not drastically impacted by the presence or absence of N-glycans, while the effector functions are impacted significantly by the IgG N-glycans present on anti-VlsE IgG during ALD. Lastly, the TLD ADCC induction followed similar trends to that of ALD, but without a significant increase in ADCC following sialidase digestion. All in all, the ADCC assay of sialidase-digested ALD confirmed that the sialic acid on the Fc region of VlsE-specific IgG reduces the immune system's ability to signal for ADCC clearance of Bb bacteria during an acute Lyme disease infection.

### Example 4-5: Sialic Acid Content on Rb-Specific IgG Modestly Impairs Antibody-Dependent Complement Deposition

Sialic acid on the Fc region of IgG interacts with complement via C1q during the initiation of the classical complement deposition pathway. Because complement deposition and the subsequent formation of the membrane attack complex is an important method for the immune system to combat bacterial infections, the present study determined how the sialic acid on anti-VlsE IgG impacts antigen-specific complement deposition (ADCD). The present study used the same exoglycosidase digestion approach as in the ADCC assay with the additional step of purifying IgG and formed Bb-specific immunocomplexes on red fluorescent streptavidin beads conjugated to biotinylated VlsE. The present study then assessed the rate of complement deposition by quantifying the abundance of C3 deposited on the red fluorescence beads using flow cytometry (FIG. 19B). The portion of beads bound by the FITC labeled anti-C3 antibody indicated complement deposition and the data was processed according to the gating strategy detailed in FIG. 19A following the protocol developed by Fischinger et al. (Journal of immunological methods, 2019. 473: p. 112630-112630).

We observed a low baseline level of complement deposition in the PBS background and LEHC controls which the present study attributes to non-specific complement deposition. This background is denoted with the dotted line (FIG. 19C). Within the mock-digested sets, the present study observed ALD cohorts deposited significantly more cleaved C3 compared to the PBS and LEHC sets, with TLD cohorts displaying a similar trend. PNGase F digestion reduced the amount of complement deposition in the ALD and TLD cohorts, reflecting the integral role N-glycans on anti-VlsE IgG play during ADCD. Lastly, the present study observed ALD VlsE-specific IgG gained an average of 11% more complement deposition following sialidase digestion, while the TLD gained an additional 7%. Taken together, the present study observed that VlsE-specific IgG lacking sialic acid gained a modest increase in the ability to induce complement deposition.

### Example 4-6

IgG and IgM N-glycomes offer potential biomarkers of active disease and provide insight into the immunological consequences for the host response to acute infection. Here, we report that acute Lyme disease (ALD) cohorts contain significantly higher levels of sialic acid in total IgG, total IgM, and anti-OspC/VlsE immunoglobulins. Moreover, the present study demonstrates that the immunological function of ADCC and ADCD are significantly improved following in vitro enzymatic removal of sialic acid from anti-VlsE IgG.

The present study sought to determine if machine learning analysis of total IgG and total IgM N-glycomes could improve discrimination of ALD from healthy controls or other diseases. After analyzing the 71 raw N-glycan features with various machine learning approaches, the present study reports that the random forest model best discriminated ALD from LEHC, ALD from TLD, and ALD from other diseases: fibromyalgia, syphilis, rheumatoid arthritis, and lupus. The present study also found that incorporating N-glycans from total IgG and total IgM improves the diagnostic performance of the Lyme disease IgM western immunoblot. To further improve the performance of the machine learning approach, the present study transformed the raw N-glycan features by pairwise log ratio to create an additional 1,231 features. Evaluating the contribution of this log-ratio transformation across machine learning methods, the improvement in ALD discrimination was slight, and most apparent in the comparison of ALD with other mimic diseases. Thus, the present study concluded that pairwise ratio transformation may serve as a technique to discover novel biomarkers in certain cohorts, but non-transformed data should also be included in the analysis.

The most significant result from the random forest analysis of total IgG and total IgM N-glycans was the shift in N-glycans between ALD and TLD cohorts. Paired t-tests revealed that the ALD cohort contained significantly higher levels of mono- and di-sialylated N-glycan species. Furthermore, the present study observed a significant improvement in the discrimination of ALD from TLD when total IgG and total IgM N-glycomes were combined with the C6 peptide ELISA and Lyme disease IgM western immunoblot results. The present study also noted that the increased sialic acid content in ALD immunoglobulins helped to differentiate ALD from rheumatoid arthritis, lupus, fibromyalgia, and syphilis. Taken together, the machine learning analysis revealed sialic acid on immunoglobulins as a potential biomarker of the immune response associated with ALD.

The Bb antigens OspC and VlsE were selected for the GlycoLyme lectin multiplex assay because they have been an industry standard for previous iterations of Lyme disease diagnostics and these antigens in combination bound the highest abundance of IgG from ALD cohorts during initial in-house optimization. The trend of ALD immunoglobulins containing higher levels of sialic acid was confirmed independently in both anti-OspC and anti-VIsE immunoglobulin populations, suggesting that multiple plasma blast cell populations are increasing the sialylation independently during ALD.

The biotinylated lectin SNA was selected because it binds predominantly to the a 2-6 linked sialic acid on complex-N-type glycans. The present study observed the trend of increased sialic acid from total IgG and total IgM N-glycans was further enhanced when examining Bb-specific immunoglobulins. High levels of sialic acid on IgG are known to reduce immune responses such as ADCC and ADCD resulting in impaired immune responses. For example, many acute infections including SARS-CoV-2, tuberculosis, pediatric meningococcal sepsis, and dengue virus reduce levels of sialic acid on the IgG Fc region. By reducing the sialic acid content, these IgG responses induce downstream immune functions to help combat the pathogen. Conversely, IgG N-glycan sialic acid increases in populations diagnosed with certain cancers including renal cell carcinoma, castration-resistant prostate cancer, precancerous advanced colonic adenomas, and smoldering myeloma as well as during pregnancy. This increased level of sialic acid on immunoglobulins suggests an immune system tolerant to malignant cells or fetal derived cellular material respectively. Suffice it to say, it was surprising to observe the increased sialic acid on Bb specific immunoglobulins during acute Lyme disease. However, this finding was supported by the study of acute Lyme disease, where increased sialic acid content in total IgG using HPLC analysis methods was observed.

The convalescent TLD time point provides compelling evidence that the high levels of sialic acid during ALD decrease in the months after infection. 90% of the TLD cohort reported that the initial antibiotic therapy resolved their symptoms. For the remaining 10% of TLD patients who reported the initial antibiotic therapy did not resolve their symptoms, 60% of those patients completed an additional course of doxycycline before donating sera at the TLD time point. Furthermore, 10 to 20% of Lyme disease patients report the initial round of antibiotic therapy did not resolve their Lyme disease symptoms. The GlycoLyme approach could determine if these patients' Bb specific immunoglobulin N-glycosylation reflects a prolonged “acute-like” response.

In addition to the GlycoLyme lectin multiplex assay identifying ALD patients specifically due to heightened sialic acid content, the present study demonstrated the role of immune inhibition that IgG N-glycan sialic acid plays using in vitro assays. The present study proceeded with only the VlsE antigen in the functional immunology experiments because Osp C inhibits complement deposition. The present study enzymatically removed sialic acid to determine the impact of sialic acid from anti-VlsE IgG on antibody-dependent cellular cytotoxicity (ADCC) and antibody-dependent complement deposition (ADCD). Without sialic acid on anti-VlsE IgG, the present study detected significantly higher induction of FcgRIIIa-dependent ADCC and a modest increase in complement deposition in the ALD cohort. Considering the various complement-evading proteins expressed on the surface of Bb, the present study concludes that ADCD impairment from host glycosylation is less important to bacterial clearance compared to the impairment of ADCC by host glycosylation during ALD. In the context of the acute bacterial infection with Bb, the impairment of ADCC could help explain why prompt antibiotic therapy is crucial to treating the infection.

Immunoglobulin N-glycosylation was not previously analyzed to determine if acute Lyme disease patients contained specific sugar biomarkers. The present study identified increased levels of sialic acid N-glycans present on IgG and IgM as important indicators of Lyme disease acuity. The present study then developed a Bb-antigen-specific lectin multiplex assay to quantitate sialic acid content associated specifically with acute Lyme disease. Following acute Lyme disease diagnosis, prompt antibiotic therapy, and three months of convalescence, the abundance of Bb-specific sialic acid significantly decreased on average 27%. The present study go on to demonstrate that acute Lyme disease patients have an impaired antibody-dependent cellular cytotoxicity and complement deposition response due to high levels of sialic acid on IgG.

### Enumerated Embodiments

In some aspects, the present invention is directed to the following non-limiting embodiments:

Embodiment 1: A method of treating, ameliorating and/or preventing acute Lyme disease in a subject, comprising: determining a glycosylation profile of a protein in the subject, wherein a change of the glycosylation profile of the protein relative to a normal glycosylation profile is associated with Lyme disease; comparing the glycosylation profile of the protein in the subject with a predetermined first glycosylation profile indicating acute Lyme disease, or a predetermined second glycosylation profile indicating a state other than acute Lyme disease; and administering to the subject a compound for treating, ameliorating and/or preventing acute Lyme disease if the determined glycosylation profile indicates acute Lyme disease.

Embodiment 2: The method of Embodiment 1, wherein the change of the protein glycosylation profile comprises an increased level of glycosylation, or a decreased level of glycosylation.

Embodiment 3: The method of any one of Embodiments 1-2, wherein the method comprises: (a) determining a glycosylation profile of one or more purified proteins in the subject, and comparing the determined glycosylation profile with a first glycosylation profile of the one or more purified proteins indicating acute Lyme disease or a second glycosylation profile of the one or more purified proteins indicating states other than acute Lyme disease; or (b) determining a total serum glycosylation profile in the subject, and comparing the determined total serum glycosylation profile with a first total serum glycosylation profile indicating acute Lyme disease or a predetermined second total serum glycosylation profile indicating states other than acute Lyme disease.

Embodiment 4: The method of any one of Embodiments 1-3, wherein the determined glycosylation profile, the first glycosylation profile and the second glycosylation profile comprise glycosylation profiles of a total serum, a total IgG, a total IgM, a Lyme-specific IgG, or a Lyme-specific IgM.

Embodiment 5: The method of any one of Embodiments 1-4, wherein at least one of the following applies:
(I) the determined glycosylation profile, the first glycosylation profile and the second glycosylation profile comprise glycosylation profiles of a total serum or a purified IgG, and the change of the glycosylation profile comprises: (a) a decreased level of terminal agalactosylated and/or fucosylated structures, and/or (b) an increase in tri- or tetra-antennary, terminal galactose or sialic acid structures;
(II) the determined glycosylation profile, the first glycosylation profile and the second glycosylation profile comprises glycosylation profiles of purified IgM, and wherein the change of the glycosylation profile comprises (a) an increase in mannose, G0, G1 or G2, and/or (b) a decrease in bisecting and sialic acid containing structures.

Embodiment 6: The method of any one of Embodiment 1-5, wherein the change of the glycosylation profile comprises at least one selected from the following:
(a) at least one IgG N-glycan selected from the group consisting of G0, G1, total G1, FA2G1 (1,6), core-fucosylated N-glycans, and FA2BG2S2 decreases in comparison with the normal IgG N-glycan;
(b) at least one IgG N-glycan selected from the group consisting of total G2, S2, FA1, FA2BG1 (1,3), FA2BG2, A12G2+Man 6, A2G2S1 (1,6), FA2G1S1 (1,6), A2G2S1 (1,3), FA2G2S1 (1,6), A2BG2S2, FA2BG2S2, and A2G2S2 (1,6) increases in comparison with the normal IgG N-glycan;
(c) at least one IgM N-glycan selected from the group consisting of total G2, FA2BG1S1 (1,3), S2, FA2BG2S2, bisecting N-glycans, A2G2S2 (3,6), and A2G2S2 (6,6) decreases in comparison with the normal IgM N-glycan;
(d) at least one IgM N-glycan selected from the group consisting of total G1, G1, G2, Total mannose N-glycans, Man 5, M4G1S1+A3G2, A1, FA1, G0, FA2G1, M4G1, FM4A1, M4A1G1, and Man 6 D1 or D2 increases in comparison with the normal IgM N-glycan;
(e) at least one total serum N-glycan selected from the group consisting of core-fucosylated N-glycans, G0, total G1, G1, FA2G1 (1,6), FA2G1 (1,3). FA2BG1 (1,6), G2, and FA2BG2 decreases in comparison with the normal total serum N-glycan;
(f) at least one total serum N-glycan selected from the group consisting of S2 and A2G2S2 (1,3) increases in comparison with the normal total serum N-glycan.

Embodiment 7: The method of any one of Embodiments 1-5, wherein the determined glycosylation profile of the protein in the subject is compared with at least one of a glycosylation profile indicating a healthy state, a glycosylation profile indicating a state of acute Lyme disease, a glycosylation profile indicating disseminated Lyme disease, a glycosylation profile indicating a state of recovering from Lyme disease, a glycosylation profile indicating a recovered case of Lyme disease, and a glycosylation profile indicating a non-Lyme infection or inflammatory disease.

Embodiment 8: The method of any one of Embodiments 1-7, wherein the method further comprises comparing the determined glycosylation profile with a glycosylation profile indicating a non-Lyme infection or inflammatory disease to exclude the non-Lyme infection or inflammatory disease, wherein the non-Lyme infection or inflammatory disease comprises at least one selected from the group consisting of fibromyalgia, lupus rheumatoid arthritis, and syphilis.

Embodiment 9: The method of any one of Embodiments 1-8, wherein the method determines the stage of Lyme disease in addition to the absence or presence of acute Lyme disease.

Embodiment 10: The method of Embodiment 9, wherein the stage of Lyme disease includes early localized or acute Lyme disease, early disseminated Lyme disease, late disseminated Lyme disease, state of recovering from Lyme disease, or reinfection with Lyme disease.

Embodiment 11: The method of any one of Embodiments 1-10, further comprises diagnosing the acute Lyme disease with the detection of Borrelia reactive antibodies in the subject.

Embodiment 12: The method of any one of Embodiments 1-11, wherein determining the glycosylation profile of the protein in the subject comprises at least one selected from the group consisting of: purifying the protein from a sample of the subject and releasing glycans from the protein, and releasing glycans from total serum proteins.

Embodiment 13: The method of any one of Embodiments 1-12, wherein the glycosylation profile of the protein in the subject and/or the predetermined glycosylation profiles are determined by:
(a) a mass spectrometry method selected from the group consisting of: matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, matrix-assisted laser desorption/ionization imaging Fourier transform ion cyclotron resonance (MALDI-FT-ICR) mass spectrometry, Quadrupole ion trap (QIT) mass spectrometry, Linear Ion Trap (LIT) mass spectrometry, Orbitrap mass spectrometry, Magnetic sector mass analyzer;
(b) at least one selected from the group consisting of a high-pressure liquid chromatography (HIPLC), and an ultra-low pressure liquid chromatography (UPLC);
(c) a capillary electrophoresis-based systems (CE);
(d) a microchip-based systems; and/or
(e) a lectin or enzyme-linked immunosorbent assay (FLISA/ELISA)-based method, a microchip array method, or a western blotting method.

Embodiment 14: The method of any one of Embodiments 1-13, wherein the method diagnoses an acute Lyme disease in the subject before or after seroconversion.

Embodiment 15: The method of Embodiment 1-14, wherein the compound comprises an antibiotic effective for killing a Borrelia bacteria.

Embodiment 16: The method of Embodiment 15, wherein the antibiotic comprises at least one selected from the group consisting of doxycycline, amoxicillin, a cephalosporin, and azithromycin.

Embodiment 17: The method of any one of Embodiments 15-16, wherein the antibiotic is administered orally or parentally.

Embodiment 18: The method of any one of Embodiments 1-17, wherein comparing the glycosylation profile is combined with a C6 peptide ELISA or a Lyme disease IgM western immunoblot results when determining whether the subject is suffering from acute Lyme disease.

Embodiment 19: The method of Embodiment 18, wherein the glycosylation profile comprises total IgG and total IgM N-glycomes.

Embodiment 20: The method of any one of Embodiments 1-19, wherein the subject is a mammal, optionally a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating or preventing acute Lyme disease in a subject, comprising:
determining a glycosylation profile of a protein in the subject, wherein a change of the glycosylation profile of the protein relative to a normal glycosylation profile is associated with Lyme disease;
comparing the glycosylation profile of the protein in the subject with a predetermined first glycosylation profile indicating acute Lyme disease, or a predetermined second glycosylation profile indicating a state other than acute Lyme disease; and
administering to the subject a compound for treating, ameliorating or preventing acute Lyme disease if the determined glycosylation profile indicates acute Lyme disease.

2. The method of claim 1, wherein the change of the protein glycosylation profile comprises an increased level of glycosylation, or a decreased level of glycosylation.

3. The method of claim 1, wherein the method comprises:
(a) determining a glycosylation profile of one or more purified proteins in the subject, and comparing the determined glycosylation profile with a first glycosylation profile of the one or more purified proteins indicating acute Lyme disease or a second glycosylation profile of the one or more purified proteins indicating states other than acute Lyme disease; or
(b) determining a total serum glycosylation profile in the subject, and comparing the determined total serum glycosylation profile with a first total serum glycosylation profile indicating acute Lyme disease or a predetermined second total serum glycosylation profile indicating states other than acute Lyme disease.

4. The method of claim 1, wherein the determined glycosylation profile, the first glycosylation profile and the second glycosylation profile comprise glycosylation profiles of a total serum, a total IgG, a total IgM, a Lyme-specific IgG, or a Lyme-specific IgM.

5. The method of claim 1, wherein at least one of the following applies:
(I) the determined glycosylation profile, the first glycosylation profile and the second glycosylation profile comprise glycosylation profiles of a total serum or a purified IgG, and the change of the glycosylation profile comprises: (a) a decreased level of terminal agalactosylated or fucosylated structures, or (b) an increase in tri- or tetra-antennary, terminal galactose or sialic acid structures;
(II) the determined glycosylation profile, the first glycosylation profile and the second glycosylation profile comprises glycosylation profiles of purified IgM, and wherein the change of the glycosylation profile comprises (a) an increase in mannose, G0, G1 or G2, or (b) a decrease in bisecting and sialic acid containing structures.

6. The method of claim 1, wherein the change of the glycosylation profile comprises at least one selected from the following:
(a) at least one IgG N-glycan selected from the group consisting of G0, G1, total G1, FA2G1 (1,6), core-fucosylated N-glycans, and FA2BG2S2 decreases in comparison with the normal IgG N-glycan;
(b) at least one IgG N-glycan selected from the group consisting of total G2, S2, FA1, FA2BG1 (1,3), FA2BG2, A12G2+Man 6, A2G2S1 (1,6), FA2G1S1 (1,6), A2G2S1 (1,3), FA2G2S1 (1,6), A2BG2S2, FA2BG2S2, and A2G2S2 (1,6) increases in comparison with the normal IgG N-glycan;
(c) at least one IgM N-glycan selected from the group consisting of total G2, FA2BG1S1 (1,3), S2, FA2BG2S2, bisecting N-glycans, A2G2S2 (3,6), and A2G2S2 (6,6) decreases in comparison with the normal IgM N-glycan;
(d) at least one IgM N-glycan selected from the group consisting of total G1, G1, G2, Total mannose N-glycans, Man 5, M4G1S1+A3G2, A1, FA1, G0, FA2G1, M4G1, FM4A1, M4A1G1, and Man 6 D1 or D2 increases in comparison with the normal IgM N-glycan;
(e) at least one total serum N-glycan selected from the group consisting of core-fucosylated N-glycans, G0, total G1, G1, FA2G1 (1,6), FA2G1 (1,3). FA2BG1 (1,6), G2, and FA2BG2 decreases in comparison with the normal total serum N-glycan;
(f) at least one total serum N-glycan selected from the group consisting of S2 and A2G2S2 (1,3) increases in comparison with the normal total serum N-glycan.

7. The method of claim 1, wherein the determined glycosylation profile of the protein in the subject is compared with at least one of a glycosylation profile indicating a healthy state, a glycosylation profile indicating a state of acute Lyme disease, a glycosylation profile indicating disseminated Lyme disease, a glycosylation profile indicating a state of recovering from Lyme disease, a glycosylation profile indicating a recovered case of Lyme disease, and a glycosylation profile indicating a non-Lyme infection or inflammatory disease.

8. The method of claim 1, wherein the method further comprises comparing the determined glycosylation profile with a glycosylation profile indicating a non-Lyme infection or inflammatory disease to exclude the non-Lyme infection or inflammatory disease, wherein the non-Lyme infection or inflammatory disease comprises at least one selected from the group consisting of fibromyalgia, lupus rheumatoid arthritis, and syphilis.

9. The method of claim 1, wherein the method determines the stage of Lyme disease in addition to the absence or presence of acute Lyme disease.

10. The method of claim 9, wherein the stage of Lyme disease includes early localized or acute Lyme disease, early disseminated Lyme disease, late disseminated Lyme disease, state of recovering from Lyme disease, or reinfection with Lyme disease.

11. The method of claim 1, further comprises diagnosing the acute Lyme disease with the detection of Borrelia reactive antibodies in the subject.

12. The method of claim 1, wherein determining the glycosylation profile of the protein in the subject comprises at least one selected from the group consisting of: purifying the protein from a sample of the subject and releasing glycans from the protein, and releasing glycans from total serum proteins.

13. The method of claim 1, wherein the glycosylation profile of the protein in the subject or the predetermined glycosylation profiles are determined by:
(a) a mass spectrometry method selected from the group consisting of: matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, matrix-assisted laser desorption/ionization imaging Fourier transform ion cyclotron resonance (MALDI-FT-ICR) mass spectrometry, Quadrupole ion trap (QIT) mass spectrometry, Linear Ion Trap (LIT) mass spectrometry, Orbitrap mass spectrometry, Magnetic sector mass analyzer;
(b) at least one selected from the group consisting of a high-pressure liquid chromatography (HPLC), and an ultra-low pressure liquid chromatography (UPLC);
(c) a capillary electrophoresis-based systems (CE);
(d) a microchip-based systems; or
(e) a lectin or enzyme-linked immunosorbent assay (FLISA/ELISA)-based method, a microchip array method, or a western blotting method.

14. The method of claim 1, wherein the method diagnoses an acute Lyme disease in the subject before or after seroconversion.

15. The method of claim 1, wherein the compound comprises an antibiotic effective for killing a Borrelia bacteria.

16. The method of claim 15, wherein the antibiotic comprises at least one selected from the group consisting of doxycycline, amoxicillin, a cephalosporin, and azithromycin.

17. The method of claim 15, wherein the antibiotic is administered orally or parentally.

18. The method of claim 1, wherein comparing the glycosylation profile is combined with a C6 peptide ELISA or a Lyme disease IgM western immunoblot results when determining whether the subject is suffering from acute Lyme disease.

19. The method of claim 18, wherein the glycosylation profile comprises total IgG and total IgM N-glycomes.

20. The method of claim 1, wherein the subject is a mammal, optionally a human.
